(19) United States
(12) Patent Application Publication
(10) Pub. No.: US 2006/0183763 A1
(43) Pub. Date: Aug. 17, 2006

(54) Novel pyrrolidyl derivatives of heteroaromatic compounds

(75) Inventors: Thomas A. Chappie, Old Lyme, CT (US); John M. Humphrey, Mystic, CT (US)

Correspondence Address: PFIZER INC, 150 EAST 42ND STREET, 5TH FLOOR - STOP 49, NEW YORK, NY, 10017-5612, US

(73) Assignee: Pfizer Inc

(21) Appl. No.: 11/257,179

(22) Filed: Oct. 24, 2005

Related U.S. Application Data
(60) Provisional application No. 60/640,405, filed on Dec. 31, 2004.

(51) Int. Cl. A61K 31/517; C07D 403/02

(52) U.S. Cl. 514/266.2; 544/284; 514/266.21

(57) ABSTRACT

The invention pertains to new pyrrolidyl derivatives of benzo-fused aza heteroaromatic compounds that serve as effective phosphodiesterase (PDE) inhibitors. The invention also relates to compounds that are selective inhibitors of PDE-10. The invention further relates to intermediates for preparation of such compounds; pharmaceutical compositions comprising such compounds; and the use of such compounds in methods for treating certain central nervous system (CNS) or other disorders. The invention relates also to methods for treating neurodegenerative and psychiatric disorders, for example psychosis and disorders comprising deficient cognition as a symptom.

### CROSS REFERENCE TO RELATED APPLICATION

The present application claims benefit of U.S. Ser. No. 60/640,405 filed on Dec. 31, 2004 which is incorporated by reference herein in its entirety.

### FIELD OF THE INVENTION

The invention pertains to new pyrrolidyl derivatives of benzo-fused aza heteroaromatic compounds that serve as effective phosphodiesterase (PDE) inhibitors. The invention also relates to compounds that are selective inhibitors of PDE-10. The invention further relates to intermediates for preparation of such compounds; pharmaceutical compositions comprising such compounds; and the use of such compounds in methods for treating certain central nervous system (CNS) or other disorders. The invention relates also to methods for treating neurodegenerative and psychiatric disorders, for example psychosis and disorders comprising deficient cognition as a symptom.

### BACKGROUND OF INVENTION

Phosphodiesterases (PDEs) are a class of intracellular enzymes involved in the hydrolysis of the nucleotides cyclic adenosine monophosphate (cAMP) and cyclic guanosine monophosphates (cGMP) into their respective nucleotide monophosphates. The cyclic nucleotides cAMP and cGMP are synthesized by adenylyl and guanylyl cyclases, respectively, and serve as secondary messengers in several cellular pathways.

The cAMP and cGMP function as intracellular second messengers regulating a vast array of intracellular processes particularly in neurons of the central nervous system. In neurons, this includes the activation of cAMP and cGMP-dependent kinases and subsequent phosphorylation of proteins involved in acute regulation of synaptic transmission as well as in neuronal differentiation and survival. The complexity of cyclic nucleotide signaling is indicated by the molecular diversity of the enzymes involved in the synthesis and degradation of cAMP and cGMP. There are at least ten families of adenylyl cyclases, two of guanylyl cyclases, and eleven of phosphodiesterases. Furthermore, different types of neurons are known to express multiple isozymes of each of these classes, and there is good evidence for compartmentalization and specificity of function for different isozymes within a given neuron.

A principal mechanism for regulating cyclic nucleotide signaling is by phosphodiesterase-catalyzed cyclic nucleotide catabolism. There are 11 known families of PDEs encoded by 21 different genes. Each gene typically yields multiple splice variants that further contribute to the isozyme diversity. The PDE families are distinguished functionally based on cyclic nucleotide substrate specificity, mechanism(s) of regulation, and sensitivity to inhibitors. Furthermore, PDEs are differentially expressed throughout the organism, including in the central nervous system. As a result of these distinct enzymatic activities and localization, different PDEs' isozymes can serve distinct physiological functions. Furthermore, compounds that can selectively inhibit distinct PDE families or isozymes may offer particular therapeutic effects, fewer side effects, or both.

PDE10 is identified as a unique family based on primary amino acid sequence and distinct enzymatic activity. Homology screening of EST databases revealed mouse PDE10A as the first member of the PDE10 family of PDEs (Fujishige et al., J. Biol. Chem. 274:18438-18445, 1999; Loughney, K. et al., Gene 234:109-117, 1999). The murine homologue has also been cloned (Soderling, S. et al., Proc. Natl. Acad. Sci. USA 96:7071-7076, 1999)and N-terminal splice variants of both the rat and human genes have been identified (Kotera, J. et al., Biochem. Biophys. Res. Comm. 261:551-557, 1999; Fujishige, K. et al., Eur. J. Biochem. 266:1118-1127, 1999). There is a high degree of homology across species. The mouse PDE10A1 is a 779 amino acid protein that hydrolyzes both cAMP and cGMP to AMP and GMP, respectively. The affinity of PDE10 for cAMP (Km=0.05 μM) is higher than for cGMP (Km=3 μM). However, the approximately 5-fold greater Vmax for cGMP over cAMP has lead to the suggestion that PDE10 is a unique cAMP-inhibited cGMPase (Fujishige et al., J. Biol. Chem. 274:18438-18445, 1999).

PDE10 also is uniquely localized in mammals relative to other PDE families. mRNA for PDE10 is highly expressed only in testis and brain (Fujishige, K. et al., Eur J Biochem. 266:1118-1127, 1999; Soderling, S. et al., Proc. Natl. Acad. Sci. 96:7071-7076, 1999; Loughney, K. et al., Gene 234:109-117, 1999). These initial studies indicated that within the brain PDE10 expression is highest in the striatum (caudate and putamen), n. accumbens, and olfactory tubercle. More recently, a detailed analysis has been made of the expression pattern in rodent brain of PDE10 mRNA (Seeger, T. F. et al., Abst. Soc. Neurosci. 26:345.10, 2000) and PDE10 protein (Menniti, F. S., Stick, C. A., Seeger, T. F., and Ryan, A. M., Immunohistochemical localization of PDE10 in the rat brain. William Harvey Research Conference ‘Phosphodiesterase in Health and Disease’, Porto, Portugal, Dec. 5-7, 2001).

United States Patent Application Publication No. 2003/0032579 discloses a method for treating certain neurologic and psychiatric disorders with the selective PDE10 inhibitor papaverine. In particular, the method relates to psychotic disorders such as schizophrenia, delusional disorders and drug-induced psychosis; to anxiety disorders such as panic and obsessive-compulsive disorder; and to movement disorders including Parkinson's disease and Huntington's disease.

In their role as second messengers in intracellular signaling events, cAMP and cGMP affect a wide array of processes including neurotransmission and enzyme activation. Intracellular levels of these chemicals are largely maintained by two classes of enzymes in response to other cellular stimuli. The first of these enzymes, the adenylyl and guanylyl cyclases, catalyze the formation of cAMP and cGMP thereby raising their concentrations and activating certain signaling events. The second enzyme class, the phosphodiesterases (PDE's), catalyzes the degradation of cAMP and cGMP, which results in termination of the signal.

Signal enhancement via elevation of cyclic nucleotide concentration can be induced through employment of PDE inhibitors. The present invention describes the use of such PDE inhibitors as therapies for the prevention or treatment of diseases linked to abnormal cell signaling processes, and relates to compounds described below.

### SUMMARY OF THE INVENTION

In one aspect, the invention relates to compounds having the following formula, denoted herein as formula I: 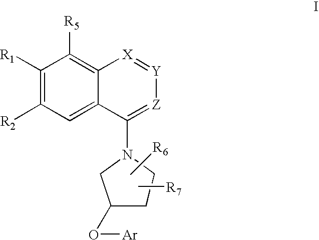
or a pharmaceutically acceptable salt, solvate or prodrug thereof,

wherein X, Y and Z are each independently CH or N with the proviso that at least one or two of X, Y and Z are N, but not all three, and with the proviso that Y and Z are not both N;

wherein R_{1,} R₂ and R₅ are independently H, halogen, C≡N, —COOH, —COOR₃, —CON R₃R₄, COR₃, —NR₃R₄, —NHCOR₃, —OH, (C₆-C₁₀)aryl, 5 to 7 membered heteroaryl, (C₁-C₆)alkyl, (C₁-C₆)haloalkyl (C₂-C₆)alkenyl, (C₂-C₆)alkynyl, —O—(C₁-C₆)alkyl, —O—(C₂-C₆)alkenyl or (C₃-C₈) cycloalkyl; or, when R₁, R₂ and R₅ are independently —O—(C₁-C₆)alkyl, —O—(C₂-C₆)alkenyl, (C₁-C₆)alkyl, (C₂-C₆)alkenyl or (C₂-C₆)alkynyl, R₁ and R₂ or R₁ and R₅ may optionally be connected to form a 5 to 8 membered ring;

wherein R₃ and R₄ are independently H, (C₁-C₆)alkyl or (C₆-C₁₀)aryl said aryl optionally substituted with one or more (C₁-C₆)alkyl groups;

wherein R₆ and R₇ are each independently H, halogen, —COOR₃, —CONR₃R₄, —COR₄, NR₃R₄, —NHCOR₃, —OH, —(C₁-C₆)alkylene-OH, —HNCOOR₃, —CN, —HNCONHR₄, (C₁-C₆)alkyl, (C₂-C₆)alkoxy, C₆-C₁₀ aryl or 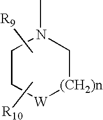
wherein n is 0 or 1;

W is carbon, oxygen or NR₈, wherein R₈ is hydrogen or (C₁-C₆)alkyl, and when W is carbon, it may be optionally substituted by halogen, —C≡N, —COOH, —COOR₃, —CONR₃R₄, —COR₃, —NR₃R₄, —NHCOR₃, —OH, (C₆-C₁₀)aryl, 5 to 7 membered heteroaryl, (C₁-C₆)alkyl, (C₁-C₆)haloalkyl (C₂-C₆)alkenyl, (C₂-C₆)alkynyl, —O—(C₁-C₆)alkyl, —O—(C₂-C₆)alkenyl or (C₃-C₈) cycloalkyl;

wherein R₉ and R₁₀ are independently hydrogen or (C₁-C₈)alkyl;

or R₉ and R₁₀ may optionally combine to form a cyclic ring;

wherein Ar is phenyl, naphthyl, or a 5- to 6-membered heteroaryl ring, which heteroaryl is optionally fused to a benzo group, and which heteroaryl contains from one to four heteroatoms selected from oxygen, nitrogen and sulfur, with the proviso that said heteroaryl ring cannot contain two adjacent oxygen atoms or two adjacent sulfur atoms, and wherein each of the foregoing phenyl, naphthyl, heteroaryl, or benzo-fused heteroaryl rings may optionally be substituted with from one to three substituents independently selected from (C₁-C₈)alkyl, chloro-, bromo-, iodo, fluoro-, (C₁-C₈)hydroxyalkyl-, (C₁-C₈)alkoxy-(C₁-C₈)alkyl-, (C₃-C₈)hydroxycycloalkyl-, (C₃-C₈)cycloalkyl, (C₃-C₈)cycloalkoxy-, (C₁-C₈)alkoxy-(C₃-C₈)cycloalkyl-, (3-8 membered)heterocycloalkyl, hydroxyl(3-8 membered)heterocycloalkyl, and (C₁-C₈)alkoxy-(3-8 membered)heterocycloalkyl, wherein said alkyl, alkoxy and cycloalkyl may be optionally substituted with 1 to 3 halos and wherein each (C₃-C₈)cycloalkyl or heterocycloalkyl moiety may be independently substituted with from one to three (C₁-C₆)alkyl or benzyl groups; or

wherein Ar is a 5- to 6-membered heteroaryl ring, which heteroaryl is fused to an imidazo, pyrido, pyrimido, pyrazo, pyridazo, or pyrrolo group, and which heteroaryl contains from one to four heteroatoms selected from oxygen, nitrogen and sulfur, with the proviso that said heteroaryl ring cannot contain two adjacent oxygen atoms or two adjacent sulfur atoms, and wherein each of the foregoing fused heteroaryl rings may optionally be substituted with from one to three substituents independently selected from (C₁-C₈)alkyl, chloro-, bromo-, iodo, fluoro-, halo(C₁-C₈)alkyl, (C₁-C₈)hydroxyalkyl-, (C₁-C₈)alkoxy-(C₁-C₈)alkyl-, —O—(C₁-C₈)alkyl-halo, (C₃-C₈)hydroxycycloalkyl-, (C₃-C₈)cycloalkyl, (C₃-C₈)cycloalkoxy-, (C₁-C₈)alkoxy-(C₃-C₈)cycloalkyl-, (3-8 membered)heterocycloalkyl, hydroxyl(3-8 membered)heterocycloalkyl, and (C₁-C₈)alkoxy-(3-8 membered)heterocycloalkyl, wherein each (C₃-C₈)cycloalkyl or heterocycloalkyl moiety may be independently substituted with from one to three (C₁-C₆)alkyl or benzyl groups; or

when Ar is phenyl, naphthyl, or heteroaryl ring, each ring may be optionally substituted with one to three substituents independently selected from (a) lactone formed from —(CH₂)ₜOH with an ortho —COOH, wherein t is one, two or three; (b) —CONR₁₄R₁₅, wherein R₁₄ and R₁₅ are independently selected from (C₁-C₈)alkyl and benzyl, or R₁₄ and R₁₅ together with the nitrogen to which they are attached form a 5- to 7-membered heteroalkyl ring that may contain from zero to three heteroatoms selected from nitrogen, sulfur and oxygen in addition to the nitrogen of the —CONR₁₄R₁₅ group, wherein when any of said heteroatoms is nitrogen it may be optionally substituted with (C₁-C₈)alkyl or benzyl, with the proviso that said ring cannot contain two adjacent oxygen atoms or two adjacent sulfur atoms; or (c) —(CH₂)ᵥNCOR₁₄R₁₅ wherein v is zero, one, two or three and —COR₁₄R₁₅ taken together with the nitrogen to which they are attached form a 4- to 6-membered lactam ring.

Compounds of the Formula I may have optical centers and therefore may occur in different enantiomeric and diastereomeric configurations. The present invention includes all enantiomers, diastereomers, and other stereoisomers of such compounds of the Formula I, as well as racemic compounds and racemic mixtures and other mixtures of stereoisomers thereof.

Pharmaceutically acceptable salts of the compounds of Formula I include the acid addition and base salts thereof.

Suitable acid addition salts are formed from acids that form non-toxic salts. Examples include, but are not limited to, the acetate, adipate, aspartate, benzoate, besylate, bicarbonate/carbonate, bisulphate/sulphate, borate, camsylate, citrate, cyclamate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mandelates mesylate, methylsulphate, naphthylate, 2-napsylate, nicotinate, nitrate, orotate, oxalate, palmitate, pamoate, phosphate/hydrogen phosphate/dihydrogen phosphate, pyroglutamate, salicylate, saccharate, stearate, succinate, sulfonate, stannate, tartrate, tosylate, trifluoroacetate and xinofoate salts.

Suitable base salts are formed from bases that form non-toxic salts. Examples include, but are not limited to, the aluminium, arginine, benzathine, calcium, choline, diethylamine, diolamine, glycine, lysine, magnesium, meglumine, olamine, potassium, sodium, tromethamine and zinc salts.

Hemisalts of acids and bases may also be formed, for example, hemisulphate and hemicalcium salts.

For a review on suitable salts, see Handbook of Pharmaceutical Salts: Properties, Selection, and Use by Stahl and Wermuth (Wiley-VCH, 2002).

Pharmaceutically acceptable salts of compounds of Formula I may be prepared by one or more of three methods:

(i) by reacting the compound of Formula I with the desired acid or base;

(ii) by removing an acid- or base-labile protecting group from a suitable precursor of the compound of Formula I or by ring-opening a suitable cyclic precursor, for example, a lactone or lactam, using the desired acid or base; or

(iii) by converting one salt of the compound of Formula I to another by reaction with an appropriate acid or base or by means of a suitable ion exchange column.

All three reactions are typically carried out in solution. The resulting salt may precipitate out and be collected by filtration or may be recovered by evaporation of the solvent. The degree of ionization in the resulting salt may vary from completely ionised to almost non-ionised.

The compounds of the invention may exist in a continuum of solid states ranging from fully amorphous to fully crystalline. The term ‘amorphous’ refers to a state in which the material lacks long range order at the molecular level and, depending upon temperature, may exhibit the physical properties of a solid or a liquid. Typically such materials do not give distinctive X-ray diffraction patterns and, while exhibiting the properties of a solid, are more formally described as a liquid. Upon heating, a change from solid to liquid properties occurs which is characterised by a change of state, typically second order (‘glass transition’). The term ‘crystalline’ refers to a solid phase in which the material has a regular ordered internal structure at the molecular level and gives a distinctive X-ray diffraction pattern with defined peaks. Such materials when heated sufficiently will also exhibit the properties of a liquid, but the change from solid to liquid is characterised by a phase change, typically first order (‘melting point’).

The compounds of the invention may also exist in unsolvated and solvated forms. The term ‘solvate’ is used herein to describe a molecular complex comprising the compound of the invention and one or more pharmaceutically acceptable solvent molecules, for example, ethanol. The term ‘hydrate’ is employed when said solvent is water.

A currently accepted classification system for organic hydrates is one that defines isolated site, channel, or metal-ion coordinated hydrates—see Polymorphism in Pharmaceutical Solids by K. R. Morris (Ed. H. G. Brittain, Marcel Dekker, 1995). Isolated site hydrates are ones in which the water molecules are isolated from direct contact with each other by intervening organic molecules. In channel hydrates, the water molecules lie in lattice channels where they are next to other water molecules. In metal-ion coordinated hydrates, the water molecules are bonded to the metal ion.

When the solvent or water is tightly bound, the complex will have a well-defined stoichiometry independent of humidity. When, however, the solvent or water is weakly bound, as in channel solvates and hygroscopic compounds, the water/solvent content will be dependent on humidity and drying conditions. In such cases, non-stoichiometry will be the norm.

The compounds of the invention may also exist in a mesomorphic state (mesophase or liquid crystal) when subjected to suitable conditions. The mesomorphic state is intermediate between the true crystalline state and the true liquid state (either melt or solution). Mesomorphism arising as the result of a change in temperature is described as ‘thermotropic’ and that resulting from the addition of a second component, such as water or another solvent, is described as ‘Iyotropic’. Compounds that have the potential to form lyotropic mesophases are described as ‘amphiphilic’ and consist of molecules which possess an ionic (such as —COO⁻Na⁺, —COO⁻K⁺, or —SO₃⁻Na⁺) or non-ionic (such as —N⁻N⁺ (CH₃)₃) polar head group. For more information, see Crystals and the Polarizing Microscope by N. H. Hartshorne and A. Stuart, 4^{th} Edition (Edward Arnold, 1970).

Hereinafter all references to compounds of Formula I include references to salts, solvates, multi-component complexes and liquid crystals thereof and to solvates, multi-component complexes and liquid crystals of salts thereof.

The compounds of the invention include compounds of Formula I as hereinbefore defined, including all polymorphs and crystal habits thereof, prodrugs and isomers thereof (including optical, geometric and tautomeric isomers) as hereinafter defined and isotopically-labeled compounds of Formula I.

### DETAILED DESCRIPTION OF THE INVENTION

In one embodiment of the present invention R₁ and R₂ are each independently (C₁-C₄)alkoxy, either X and Y or X and Z are N and R₅ is H; preferably R₁ and R₂ are methoxy.

In one embodiment of the present invention Ar is phenyl optionally substituted by —O—(C₁-C₅)alkyl, —(C₁-C₅)alkyl, CN, hydroxyl, phenyl or —O—(C₁-C₅)alkyl substituted with 1 to 3 halogens. Preferably Ar is phenyl substituted by trifluoromethoxy.

In another embodiment of the present invention Ar is naphthyl or naphthyl substituted by —O—(C₁-C₅)alkyl.

In another embodiment Ar is 5 or 6 membered heteroaryl, preferably substituted or unsubstituted quinoxalinyl, isoquinolinyl or quinolinyl, and most preferably unsubstituted quinoxalinyl.

In one embodiment of the present invention R₆ or R₇ is —O—(C₁-C₆)alkyl preferably methoxy.

In another embodiment of the present invention R₆ or R₇ is —NR₃R₄, preferably R₃ and R₄ are each independently (C₁₋C₃)alkyl.

Specific compounds of the present invention are as follows:

4-[3-Allyl-4-(quinoxalin-2-yloxy)-pyrrolidin-1-yl]-6,7-dimethoxy-quinazoline;

6,7-Dimethoxy-4-[3-propyl-4-(quinoxalin-2-yloxy)-pyrrolidin-1-yl]-quinazoline;

1-(6,7-Dimethoxy-quinazolin-4-y)-3-methyl-4-(quinoxalin-2-yloxy)-pyrrolidine-3-carboxylic acid ethyl ester;

6,7-Dimethoxy-4-[3-methyl-3-(quinoxalin-2-yloxy)-pyrrolidin-1-yl]-quinazoline;

[1-(6,7-Dimethoxy-quinazolin-4-yl)-4-(quinoxalin-2-yloxy)-pyrrolidin-3-yl]-isopropyl-methyl-amine;

[1-(6,7-Dimethoxy-quinazolin-4-yl)-4-(quinoxalin-2-yloxy)-pyrrolidin-3-yl]-diethyl-amine;

[1-(6,7-Dimethoxy-quinazolin-4-yl)-4-(quinoxalin-2-yloxy)-pyrrolidin-3-yl]-ethyl-methyl-amine;

[1-(6,7-Dimethoxy-quinazolin-4-yl)-4-(quinoxalin-2-yloxy)-pyrrolidin-3-yl]-dimethyl-amine;

[1-(6,7-Dimethoxy-quinazolin-4-yl)-4-(quinolin-2-yloxy)-pyrrolidin-3-yl]-dimethyl-amine;

[1-(6,7-Dimethoxy-quinazolin-4-yl)-4-(quinolin-3-yloxy)-pyrrolidin-3-yl]-dimethyl-amine;

6,7-Dimethoxy-4-[4′-(quinoxalin-2-yloxy)-[1,3′]bipyrrolidinyl-1′-yl]-quinazoline;

6,7-Dimethoxy-4-[3-morpholin-4-yl-4-(quinoxalin-2-yloxy)-pyrrolidin-1-yl]-quinazoline;

6,7-Dimethoxy-4-[3-(4-methyl-piperazin-1-yl)-4-(quinoxalin-2-yloxy)-pyrrolidin-1-yl]-quinazoline;

[1-(6,7-Dimethoxy-quinazolin-4-yl)-4-(quinoxalin-2-yloxy)-pyrrolidin-3-yl]-methyl-amine;

N-[1-(6,7-Dimethoxy-quinazolin-4-yl)-4-(quinoxalin-2-yloxy)-pyrrolidin-3-yl]-N-methyl-acetamide;

6,7-Dimethoxy-4-[3-(quinoxalin-2-yloxy)-pyrrolidin-1-yl]-quinazoline;

4-[3-(4-Ethoxy-phenoxy)-pyrrolidin-1-yl]-6,7-dimethoxy-quinazoline;

6,7-Dimethoxy-4-[3-(naphthalen-2-yloxy)-pyrrolidin-1-yl]-quinazoline;

4-[3-(4-tert-Butyl-phenoxy)-pyrrolidin-1-yl]-6,7-dimethoxy-quinazoline;

4-[1-(6,7-Dimethoxy-quinazolin-4-yl)-pyrrolidin-3-yloxy]-benzonitrile;

6,7-Dimethoxy-4-[3-(4-trifluoromethoxy-phenoxy)-pyrrolidin-1-yl]-quinazoline;

4-[3-(3-Ethoxy-phenoxy)-pyrrolidin-1-yl]-6,7-dimethoxy-quinazoline;

4-[3-(3,4-Dimethoxy-phenoxy)-pyrrolidin-1-yl]-6,7-dimethoxy-quinazoline;

4-[3-(3-Isopropoxy-phenoxy)-pyrrolidin-1-yl]-6,7-dimethoxy-quinazoline;

4-[3-(Indan-5-yloxy)-pyrrolidin-1-yl]-6,7-dimethoxy-quinazoline;

6,7-Dimethoxy-4-[3-(quinolin-6-yloxy)-pyrrolidin-1-yl]-quinazoline;

N4-[3-(Biphenyl-3-yloxy)-pyrrolidin-1-yl]-6,7-dimethoxy-quinazoline;

6,7-Dimethoxy-4-[3-(2-methyl-quinolin-6-yloxy)-pyrrolidin-1-yl]-quinazoline;

6,7-Dimethoxy-4-[3-(7-methoxy-naphthalen-2-yloxy)-pyrrolidin-1-yl]-quinazoline;

6,7-Dimethoxy-4-[3-(6-methoxy-naphthalen-2-yloxy)-pyrrolidin-1-yl]-quinazoline;

6,7-Dimethoxy-4-[3-(quinolin-7-yloxy)-pyrrolidin-1-yl]-quinazoline;

6,7-Dimethoxy-4-[3-(naphthalen-1-yloxy)-pyrrolidin-1-yl]-quinazoline;

4-[3-(Isoquinolin-3-yloxy)-pyrrolidin-1-yl]-6,7-dimethoxy-quinazoline;

4-[3-(Isoquinolin-7-yloxy)-pyrrolidin-1-yl]-6,7-dimethoxy-quinazoline;

6,7-Dimethoxy-4-[3-(pyridin-2-yloxy)-pyrrolidin-1-yl]-quinazoline;

6,7-Dimethoxy-4-[3-(pyridin-3-yloxy)-pyrrolidin-1-yl]-quinazoline;

6,7-Dimethoxy-4-[3-(pyridin-4-yloxy)-pyrrolidin-1-yl]-quinazoline;

4-[3-(5-Chloro-pyrimidin-2-yloxy)-pyrrolidin-1-yl]-6,7-dimethoxy-quinazoline;

3-[1-(6,7-Dimethoxy-quinazolin-4-yl)-pyrrolidin-3-yloxy]-quinoxaline-6-carbonitrile acid tert-butyl ester;

6,7-Dimethoxy-4-[3-methoxy-4-(quinoxalin-2-yloxy)-pyrrolidin-1-yl]-quinazoline.tetrahydrofuran(pyrrolidin-3-yloxy]-quinoxaline.;

1-(6,7-Dimethoxy-quinazolin-4-yl)-4-(quinoxalin-2-yloxy)-pyrrolidin-3-ol;

[4-Benzyl-1-(6,7-dimethoxy-quinazolin-4-yl)-pyrrolidin-3-yl]-dimethyl-amine;

6,7-Dimethoxy-4-[3-(quinoxalin-2-yloxy)-pyrrolidin-1-yl]-cinnoline;

6,7-Dimethoxy-4-[4′-(quinoxalin-2-yloxy)-[1,3′]bipyrrolidinyl-1′-yl]-cinnoline;

[1-(6,7-Dimethoxy-cinnolin-4-yl)-4-(quinolin-2-yloxy)-pyrrolidin-3-yl]-ethyl-methyl-amine;

[1-(6,7-Dimethoxy-cinnolin-4-yl)-4-(quinolin-2-yloxy)-pyrrolidin-3-yl]-diethyl-amine;

6,7-Dimethoxy-4-[3-morpholin-4-yl-4-(quinoxalin-2-yloxy)-pyrrolidin-1-yl]-cinnoline;

[1-(6,7-Dimethoxy-cinnolin-4-yl)-4-(quinoxalin-2-yloxy)-pyrrolidin-3-yl]-diethyl-amine;

[1-(6,7-Dimethoxy-cinnolin-4-yl)-4-(quinoxalin-2-yloxy)-pyrrolidin-3-yl]-ethyl-methyl-amine;

[1-(6,7-Dimethoxy-cinnolin-4-yl)-4-(quinolin-2-yloxy)-pyrrolidin-3-yl]-dimethyl-amine;

6,7-Dimethoxy-4-[3-morpholin-4-yl-4-(quinolin-2-yloxy)-pyrrolidin-1-yl]-cinnoline;

6,7-Dimethoxy-4-[4′-(quinolin-2-yloxy)-[1,3′]bipyrrolidinyl-1′-yl]-cinnoline;

4-[3-(4a,5,6,7,8,8a-Hexahydro-quinoxalin-2-yloxy)-pyrrolidin-1-yl]-6,7-dimethoxy-quinazoline;

1-(6,7-Dimethoxy-quinazolin-4-yl)-4-(quinoxalin-2-yloxy)-pyrrolidine-2-carboxylic acid dimethylamide;

[1-(6,7-Dimethoxy-quinazolin-4-yl)-4-(quinoxalin-2-yloxy)-pyrrolidin-2-yl]-methanol hydrochloride; and

2-[1-(6,7-Dimethoxy-quinazolin-4-yl )-4-(quinoxalin-2-yloxy)-pyrrolidin-2-yl]-propan-2-ol hydrochloride.

The above listed compounds and their pharmaceutically acceptable salts, solvates, and prodrugs thereof are preferred embodiments of the subject invention.

As indicated, so-called ‘prodrugs’ of the compounds of Formula I are also within the scope of the invention. Thus certain derivatives of compounds of Formula I which may have little or no pharmacological activity themselves can, when administered into or onto the body, be converted into compounds of Formula I having the desired activity, for example, by hydrolytic cleavage. Such derivatives are referred to as ‘prodrugs’. Further information on the use of prodrugs may be found in Pro-drugs as Novel Delivery Systems, Vol. 14, ACS Symposium Series (T. Higuchi and W. Stella) and Bioreversible Carriers in Drug Design, Pergamon Press, 1987 (Ed. E. B. Roche, American Pharmaceutical Association).

Prodrugs in accordance with the invention can, for example, be produced by replacing appropriate functionalities present in the compounds of Formula I with certain moieties known to those skilled in the art as ‘pro-moieties’ as described, for example, in Design of Prodrugs by H. Bundgaard (Elsevier, 1985).

Some examples of prodrugs in accordance with the invention include, but are not limited to,

(i) where the compound of Formula I contains a carboxylic acid functionality (—COOH), an ester thereof, for example, a compound wherein the hydrogen of the carboxylic acid functionality of the compound of Formula (I) is replaced by (C₁-C₈)alkyl;

(ii) where the compound of Formula I contains an alcohol functionality (—OH), an ether thereof, for example, a compound wherein the hydrogen of the alcohol functionality of the compound of Formula I is replaced by (C₁-C₆)alkanoyloxymethyl; and

(iii) where the compound of Formula I contains a primary or secondary amino functionality (—NH₂ or —NHR where R≠H), an amide thereof, for example, a compound wherein, as the case may be, one or both hydrogens of the amino functionality of the compound of Formula I is/are replaced by (C₁-C₁₀)alkanoyl.

Further examples of replacement groups in accordance with the foregoing examples and examples of other prodrug types may be found in the aforementioned references.

Moreover, certain compounds of Formula I may themselves act as prodrugs of other compounds of Formula I.

Also included within the scope of the invention are metabolites of compounds of Formula I, that is, compounds formed in vivo upon administration of the drug. Some examples of metabolites in accordance with the invention include, but are not limited to,

(i) where the compound of Formula I contains a methyl group, an hydroxymethyl derivative thereof (—CH₃→—CH₂OH):

(ii) where the compound of Formula I contains an alkoxy group, an hydroxy derivative thereof (—OR→—OH);

(iii) where the compound of Formula I contains a tertiary amino group, a secondary amino derivative thereof (—NR¹R²→—NHR¹ or —NHR²);

(iv) where the compound of Formula I contains a secondary amino group, a primary derivative thereof (—NHR¹→—NH₂);

(v) where the compound of Formula I contains a phenyl moiety, a phenol derivative thereof (−Ph→−PhOH); and

(vi) where the compound of Formula I contains an amide group, a carboxylic acid derivative thereof (—CONH₂→COOH).

Compounds of Formula I containing one or more asymmetric carbon atoms can exist as two or more stereoisomers. Where a compound of Formula I contains an alkenyl or alkenylene group, geometric cis/trans (or Z/E) isomers are possible. Where structural isomers are interconvertible via a low energy barrier, tautomeric isomerism (‘tautomerism’) can occur. This can take the form of proton tautomerism in compounds of Formula I containing, for example, an imino, keto, or oxime group, or so-called valence tautomerism in compounds that contain an aromatic moiety. It follows that a single compound may exhibit more than one type of isomerism.

Included within the scope of the present invention are all stereoisomers, geometric isomers and tautomeric forms of the compounds of Formula I, including compounds exhibiting more than one type of isomerism, and mixtures of one or more thereof. Also included are acid addition or base salts wherein the counterion is optically active, for example, d-lactate or l-lysine, or racemic, for example, dl-tartrate or dl-arginine.

Cis/trans isomers may be separated by conventional techniques well known to those skilled in the art, for example, chromatography and fractional crystallisation.

Conventional techniques for the preparation/isolation of individual enantiomers include chiral synthesis from a suitable optically pure precursor or resolution of the racemate (or the racemate of a salt or derivative) using, for example, chiral high pressure liquid chromatography (HPLC).

Alternatively, the racemate (or a racemic precursor) may be reacted with a suitable optically active compound, for example, an alcohol, or, in the case where the compound of Formula I contains an acidic or basic moiety, a base or acid such as 1-phenylethylamine or tartaric acid. The resulting diastereomeric mixture may be separated by chromatography and/or fractional crystallization and one or both of the diastereoisomers converted to the corresponding pure enantiomer(s) by means well known to a skilled person.

Chiral compounds of the invention (and chiral precursors thereof) may be obtained in enantiomerically-enriched form using chromatography, typically HPLC, on an asymmetric resin with a mobile phase consisting of a hydrocarbon, typically heptane or hexane, containing from 0 to 50% by volume of isopropanol, typically from 2% to 20%, and from 0 to 5% by volume of an alkylamine, typically 0.1% diethylamine. Concentration of the eluate affords the enriched mixture.

When any racemate crystallises, crystals of two different types are possible. The first type is the racemic compound (true racemate) referred to above wherein one homogeneous form of crystal is produced containing both enantiomers in equimolar amounts. The second type is the racemic mixture or conglomerate wherein two forms of crystal are produced in equimolar amounts each comprising a single enantiomer.

While both of the crystal forms present in a racemic mixture have identical physical properties, they may have different physical properties compared to the true racemate. Racemic mixtures may be separated by conventional techniques known to those skilled in the art—see, for example, Stereochemistry of Organic Compounds by E. L. Eliel and S. H. Wilen (Wiley, 1994).

The present invention includes all pharmaceutically acceptable isotopically-labelled compounds of Formula I wherein one or more atoms are replaced by atoms having the same atomic number, but an atomic mass or mass number different from the atomic mass or mass number which predominates in nature.

Examples of isotopes suitable for inclusion in the compounds of the invention include, but are not limited to, isotopes of hydrogen, such as ²H and ³H, carbon, such as ¹¹C, ¹³C and ¹⁴C, chlorine, such as ³⁶Cl, fluorine, such as ¹⁸F, iodine, such as ¹²³I and ¹²⁵I, nitrogen, such as ¹³N and ¹⁵N, oxygen, such as ¹⁵O, ¹⁷O and ¹⁸O, phosphorus, such as ³²P, and sulphur, such as ³⁵S

Certain Isotopically-labelled compounds of Formula I, for example, those incorporating a radioactive isotope, are useful in drug and/or substrate tissue distribution studies. The radioactive isotopes tritium, i.e. ³H, and carbon-14, i.e. ¹⁴C, are particularly useful for this purpose in view of their ease of incorporation and ready means of detection.

Substitution with heavier isotopes such as deuterium, i.e. ²H, may afford certain therapeutic advantages resulting from greater metabolic stability, for example, increased in vivo half-life or reduced dosage requirements, and hence may be preferred in some circumstances.

Substitution with positron emitting isotopes, such as ¹¹C, ¹⁸F, ¹⁵O and ¹³N, can be useful in Positron Emission Topography (PET) studies for examining substrate receptor occupancy.

Isotopically-labeled compounds of Formula I can generally be prepared by conventional techniques known to those skilled in the art or by processes analogous to those described in the accompanying Examples and Preparations using an appropriate isotopically-labeled reagent in place of the non-labeled reagent previously employed.

Pharmaceutically acceptable solvates in accordance with the invention include those wherein the solvent of crystallization may be isotopically substituted, e.g. D₂O, d₆-acetone, d₆-DMSO.

This invention also pertains to a pharmaceutical composition for treatment of certain psychotic disorders and conditions such as schizophrenia, delusional disorders and drug induced psychosis; to anxiety disorders such as panic and obsessive-compulsive disorder; and to movement disorders including Parkinson's disease and Huntington's disease, comprising an amount of a compound of formula I effective in inhibiting PDE 10.

In another embodiment, this invention relates to a pharmaceutical composition for treating psychotic disorders and condition such as schizophrenia, delusional disorders and drug induced psychosis; anxiety disorders such as panic and obsessive-compulsive disorder; and movement disorders including Parkinson's disease and Huntington's disease, comprising an amount of a compound of formula I effective in treating said disorder or condition.

Examples of psychotic disorders that can be treated according to the present invention include, but are not limited to, schizophrenia, for example of the paranoid, disorganized, catatonic, undifferentiated, or residual type; schizophreniform disorder; schizoaffective disorder, for example of the delusional type or the depressive type; delusional disorder; substance-induced psychotic disorder, for example psychosis induced by alcohol, amphetamine, cannabis, cocaine, hallucinogens, inhalants, opioids, or phencyclidine; personality disorder of the paranoid type; and personality disorder of the schizoid type.

Examples of movement disorders that can be treated according to the present invention include but are not limited to Huntington's disease and dyskinesia associated with dopamine agonist therapy, Parkinson's disease, restless leg syndrome, and essential tremor.

Other disorders that can be treated according to the present invention are obsessive/compulsive disorders, Tourette's syndrome and other tic disorders.

In another embodiment, this invention relates to a method for treating an anxiety disorder or condition in a mammal which method comprises administering to said mammal an amount of a compound of formula I effective in inhibiting PDE 10.

This invention also provides a method for treating an anxiety disorder or condition in a mammal which method comprises administering to said mammal an amount of a compound of formula I effective in treating said disorder or condition.

Examples of anxiety disorders that can be treated according to the present invention include, but are not limited to, panic disorder; agoraphobia; a specific phobia; social phobia; obsessive-compulsive disorder; post-traumatic stress disorder; acute stress disorder; and generalized anxiety disorder.

This invention further provides a method of treating a drug addiction, for example an alcohol, amphetamine, cocaine, or opiate addiction, in a mammal, including a human, which method comprises administering to said mammal an amount of a compound of formula I effective in treating drug addiction.

This invention also provides a method of treating a drug addiction, for example an alcohol, amphetamine, cocaine, or opiate addiction, in a mammal, including a human, which method comprises administering to said mammal an amount of a compound of formula I effective in inhibiting PDE10.

A “drug addiction”, as used herein, means an abnormal desire for a drug and is generally characterized by motivational disturbances such a compulsion to take the desired drug and episodes of intense drug craving.

This invention further provides a method of treating a disorder comprising as a symptom a deficiency in attention and/or cognition in a mammal, including a human, which method comprises administering to said mammal an amount of a compound of formula I effective in treating said disorder.

This invention also provides a method of treating a disorder or condition comprising as a symptom a deficiency in attention and/or cognition in a mammal, including a human, which method comprises administering to said mammal an amount of a compound of formula I effective in inhibiting PDE10.

This invention also provides a method of treating a disorder or condition comprising as a symptom a deficiency in attention and/or cognition in a mammal, including a human, which method comprises administering to said mammal an amount of a compound of formula I effective in treating said disorder or condition.

The phrase “deficiency in attention and/or cognition” as used herein in “disorder comprising as a symptom a deficiency in attention and/or cognition” refers to a subnormal functioning in one or more cognitive aspects such as memory, intellect, or learning and logic ability, in a particular individual relative to other individuals within the same general age population. “Deficiency in attention and/or cognition” also refers to a reduction in any particular individual's functioning in one or more cognitive aspects, for example as occurs in age-related cognitive decline.

Examples of disorders that comprise as a symptom a deficiency in attention and/or cognition that can be treated according to the present invention are dementia, for example Alzheimer's disease, multi-infarct dementia, alcoholic dementia or other drug-related dementia, dementia associated with intracranial tumors or cerebral trauma, dementia associated with Huntington's disease or Parkinson's disease, or AIDS-related dementia; delirium; amnestic disorder; post-traumatic stress disorder; mental retardation; a learning disorder, for example reading disorder, mathematics disorder, or a disorder of written expression; attention-deficit/hyperactivity disorder; and age-related cognitive decline.

This invention also provides a method of treating a mood disorder or mood episode in a mammal, including a human, comprising administering to said mammal an amount of a compound of formula I effective in treating said disorder or episode.

This invention also provides a method of treating a mood disorder or mood episode in a mammal, including a human, comprising administering to said mammal an amount of a compound of formula I effective in inhibiting PDE10.

Examples of mood disorders and mood episodes that can be treated according to the present invention include, but are not limited to, major depressive episode of the mild, moderate or severe type, a manic or mixed mood episode, a hypomanic mood episode; a depressive episode with atypical features; a depressive episode with melancholic features; a depressive episode with catatonic features; a mood episode with postpartum onset; post-stroke depression; major depressive disorder; dysthymic disorder; minor depressive disorder; premenstrual dysphoric disorder; post-psychotic depressive disorder of schizophrenia; a major depressive disorder superimposed on a psychotic disorder such as delusional disorder or schizophrenia; a bipolar disorder, for example bipolar I disorder, bipolar II disorder, and cyclothymic disorder.

This invention further provides a method of treating a neurodegenerative disorder or condition in a mammal, including a human, which method comprises administering to said mammal an amount of a compound of formula I effective in treating said disorder or condition.

This invention further provides a method of treating a neurodegenerative disorder or condition in a mammal, including a human, which method comprises administering to said mammal an amount of a compound of formula I effective in inhibiting PDE10.

As used herein, and unless otherwise indicated, a “neurodegenerative disorder or condition” refers to a disorder or condition that is caused by the dysfunction and/or death of neurons in the central nervous system. The treatment of these disorders and conditions can be facilitated by administration of an agent which prevents the dysfunction or death of neurons at risk in these disorders or conditions and/or enhances the function of damaged or healthy neurons in such a way as to compensate for the loss of function caused by the dysfunction or death of at-risk neurons. The term “neurotrophic agent” as used herein refers to a substance or agent that has some or all of these properties.

Examples of neurodegenerative disorders and conditions that can be treated according to the present invention include, but are not limited to, Parkinson's disease; Huntington's disease; dementia, for example Alzheimer's disease, multi-infarct dementia, AIDS-related dementia, and Fronto temperal Dementia; neurodegeneration associated with cerebral trauma; neurodegeneration associated with stroke, neurodegeneration associated with cerebral infarct; hypoglycemia-induced neurodegeneration; neurodegeneration associated with epileptic seizure; neurodegeneration associated with neurotoxin poisoning; and multi-system atrophy.

In one embodiment of the present invention, the neurodegenerative disorder or condition comprises neurodegeneration of striatal medium spiny neurons in a mammal, including a human.

In a further embodiment of the present invention, the neurodegenerative disorder or condition is Huntington's disease.

In another embodiment, this invention provides a pharmaceutical composition for treating psychotic disorders, delusional disorders and drug induced psychosis; anxiety disorders, movement disorders, mood disorders, neurodegenerative disorders and drug addiction, comprising an amount of a compound of formula I effective in treating said disorder or condition.

In another embodiment, this invention provides a method of treating a disorder selected from psychotic disorders, delusional disorders and drug induced psychosis; anxiety disorders, movement disorders, mood disorders, and neurodegenerative disorders, which method comprises administering an amount of a compound of claim 1 effective in treating said disorder.

In another embodiment, this invention provides a method of treating the disorders above, where the disorders are selected from the group consisting of: dementia, Alzheimer's disease, multi-infarct dementia, alcoholic dementia or other drug-related dementia, dementia associated with intracranial tumors or cerebral trauma, dementia associated with Huntington's disease or Parkinson's disease, or AIDS-related dementia; delirium; amnestic disorder; post-traumatic stress disorder; mental retardation; a learning disorder, for example reading disorder, mathematics disorder, or a disorder of written expression; attention-deficit/hyperactivity disorder; age-related cognitive decline, major depressive episode of the mild, moderate or severe type; a manic or mixed mood episode; a hypomanic mood episode; a depressive episode with atypical features; a depressive episode with melancholic features; a depressive episode with catatonic features; a mood episode with postpartum onset; post-stroke depression; major depressive disorder; dysthymic disorder; minor depressive disorder; premenstrual dysphoric disorder; post-psychotic depressive disorder of schizophrenia; a major depressive disorder superimposed on a psychotic disorder comprising a delusional disorder or schizophrenia; a bipolar disorder comprising bipolar I disorder, bipolar II disorder, cyclothymic disorder, Parkinson's disease; Huntington's disease; dementia, Alzheimer's disease, multi-infarct dementia, AIDS-related dementia, Fronto temperal Dementia; neurodegeneration associated with cerebral trauma; neurodegeneration associated with stroke; neurodegeneration associated with cerebral infarct; hypoglycemia-induced neurodegeneration; neurodegeneration associated with epileptic seizure; neurodegeneration associated with neurotoxin poisoning; multi-system atrophy, paranoid, disorganized, catatonic, undifferentiated or residual type; schizophreniform disorder; schizoaffective disorder of the delusional type or the depressive type; delusional disorder; substance-induced psychotic disorder, psychosis induced by alcohol, amphetamine, cannabis, cocaine, hallucinogens, inhalants, opioids, or phencyclidine; personality disorder of the paranoid type; and personality disorder of the schizoid type.

The term “aryl”, as used herein, unless otherwise indicated, includes an organic radical derived from a univalent aromatic hydrocarbon and includes but is not limited to, phenyl, naphthyl and indenyl.

The term “alkyl”, as used herein, unless otherwise indicated, includes saturated monovalent hydrocarbon radicals having straight or branched moieties. Examples of alkyl groups include, but are not limited to, methyl, ethyl, propyl, isopropyl, and t-butyl.

The term “alkenyl”, as used herein, unless otherwise indicated, includes alkyl moieties having at least one carbon-carbon double bond wherein alkyl is as defined above. Examples of alkenyl include, but are not limited to, ethenyl and propenyl.

The term “alkynyl”, as used herein, unless otherwise indicated, includes alkyl moieties having at least one carbon-carbon triple bond wherein alkyl is as defined above. Examples of alkynyl groups include, but are not limited to, ethynyl and 2-propynyl.

The term “cycloalkyl”, as used herein, unless otherwise indicated, includes alkyl groups comprising non-aromatic saturated cyclic alkyl moieties wherein alkyl is as defined above. Examples of cycloalkyl include, but are not limited to, cyclopropyl, cyclopropylethyl, cyclopropylmethyl, cyclobutyl, cyclopentyl, cyclohexyl, and cycloheptyl.

Unless otherwise indicated, as used herein, the terms “heterocyclic” and “heterocycloalkyl” refer to non-aromatic cyclic groups containing one or more heteroatoms, preferably from one to four heteroatoms, each selected from O, S and N. “Heterobicycloalkyl” groups are non-aromatic two-ringed cyclic groups, wherein said rings share one or two atoms, and wherein at least one of the rings contains a heteroatom (O, S, or N). Heterobicycloalkyl groups for purposes of the present invention, and unless otherwise indicated, include spiro groups and fused ring groups. “Heterotricycloalkyl” groups are non-aromatic three-ringed cyclic groups, wherein said rings are fused to one another or form a spiro group (in other words, at least two of said rings share one or two atoms and the third ring shares one or two atoms with at least one of said two rings). The heterotricycloalkyl groups of the compounds of the present invention can include one or more O, S and/or N heteroatoms. In one embodiment, each ring in the heterobicycloalkyl or heterotricycloalkyl contains up to four heteroatoms (i.e. from zero to four heteroatoms, provided that at least one ring contains at least one heteroatom). The heterocycloalkyl, heterobicycloalky and heterotricycloalkyl groups of the present invention can also include ring systems substituted with one or more oxo moieties. The heterocyclic groups, including the heterobicyclic and heterotricyclic groups, may comprise double or triple bonds, e.g. heterocycloalkenyl, heterobicycloalkenyl, and heterotricycloalkenyl. Examples of non-aromatic heterocyclic groups are aziridinyl, azetidinyl, pyrrolidinyl, piperidinyl, azepinyl, piperazinyl, 1,2,3,6-tetrahydropyridinyl, oxiranyl, oxetanyl, tetrahydrofuranyl, tetrahydrothienyl, tetrahydropyranyl, tetrahydrothiopyranyl, morpholino, thiomorpholino, thioxanyl, pyrrolinyl, indolinyl, 2H-pyranyl, 4H-pyranyl, dioxanyl, 1,3-dioxolanyl, pyrazolinyl, dihydropyranyl, dihydrothienyl, dihydrofuranyl, pyrazolidinyl, imidazolinyl, imidazolidinyl, 3-azabicyclo[3.1.0]hexanyl, 3-azabicyclo[4.1.0]heptanyl, quinolizinyl, quinuclidinyl, 1,4-dioxaspiro[4.5]decyl, 1,4-dioxaspiro[4.4]nonyl, 1,4-dioxaspiro[4.3]octyl, and 1,4-dioxaspiro[4.2]heptyl.

“Heteroaryl”, as used herein, refers to aromatic groups containing one or more heteroatoms (O, S, or N), preferably from one to four heteroatoms. A multicyclic group containing one or more heteroatoms wherein at least one ring of the group is aromatic is a “heteroaryl” group. The heteroaryl groups of this invention can also include ring systems substituted with one or more oxo moieties. Examples of heteroaryl groups are pyridinyl, pyridazinyl, imidazolyl, pyrimidinyl, pyrazolyl, triazolyl, pyrazinyl, quinolyl, isoquinolyl, tetrazolyl, furyl, thienyl, isoxazolyl, thiazolyl, oxazolyl, isothiazolyl, pyrrolyl, indolyl, benzimidazolyl, benzofuranyl, cinnolinyl, indazolyl, indolizinyl, phthalazinyl, triazinyl, isoindolyl, purinyl, oxadiazolyl, thiadiazolyl, furazanyl, benzofurazanyl, benzothiophenyl, benzotriazolyl, benzothiazolyl, benzoxazolyl, quinazolinyl, quinoxalinyl, naphthyridinyl, dihydroquinolyl, tetrahydroquinolyl, dihydroisoquinolyl, tetrahydroisoquinolyl, benzofuryl, furopyridinyl, pyrolopyrimidinyl, and azaindolyl.

“Halogen” and “halo”, as used herein, includes chloro, bromo, fluoro and iodo.

“Haloalkyl” as used herein, includes alkyl groups where one or more of the hydrogen atoms are substituted by halogens. Examples of haloalkyl include, but are not limited to —CH₂F, —CHCl₂, —CF₃ and —CH₂CF₃.

“Neurotoxin poisoning” refers to poisoning caused by a neurotoxin. A neurotoxin is any chemical or substance that can cause neural death and thus neurological damage. An example of a neurotoxin is alcohol, which, when abused by a pregnant female, can result in alcohol poisoning and neurological damage known as Fetal Alcohol Syndrome in a newborn. Other examples of neurotoxins include, but are not limited to, kainic acid, domoic acid, and acromelic acid; certain pesticides, such as DDT; certain insecticides, such as organophosphates; volatile organic solvents such as hexacarbons (e.g. toluene); heavy metals (e.g. lead, mercury, arsenic, and phosphorous); aluminum; certain chemicals used as weapons, such as Agent Orange and Nerve Gas; and neurotoxic antineoplastic agents.

As used herein, the term “selective PDE10 inhibitor” refers to a substance, for example an organic molecule that effectively inhibits an enzyme from the PDE10 family to a greater extent than enzymes from the PDE 1-9 families or PDE11 family. In one embodiment, a selective PDE10 inhibitor is a substance, for example an organic molecule, having an IC₅₀ for inhibition of PDE10 that is less than or about one-half the IC₅₀ that the substance has for inhibition of any other PDE enzyme. In other words, the substance inhibits PDE10 activity to the same degree at a concentration of about one-tenth or less than the concentration required for any other PDE enzyme.

In general, a substance is considered to effectively inhibit PDE10 activity if it has an IC₅₀ of less than or about 10 μM, preferably less than or about 0.1 μM.

A “selective PDE10 inhibitor” can be identified, for example, by comparing the ability of a substance to inhibit PDE10 activity to its ability to inhibit PDE enzymes from the other PDE families. For example, a substance may be assayed for its ability to inhibit PDE10 activity, as well as PDE1, PDE2, PDE3, PDE4, PDE5, PDE6, PDE7, PDE8, PDE9, PDE11, including subtypes.

The term “treating”, as in “a method of treating a disorder”, refers to reversing, alleviating, or inhibiting the progress of the disorder to which such term applies, or one or more symptoms of the disorder. As used herein, the term also encompasses, depending on the condition of the patient, preventing the disorder, including preventing onset of the disorder or of any symptoms associated therewith, as well as reducing the severity of the disorder or any of its symptoms prior to onset. “Treating” as used herein refers also to preventing a recurrence of a disorder.

For example, “treating schizophrenia, or schizophreniform or schizoaffective disorder” as used herein also encompasses treating one or more symptoms (positive, negative, and other associated features) of said disorders, for example treating, delusions and/or hallucination associated therewith. Other examples of symptoms of schizophrenia and schizophreniform and schizoaffecctive disorders include disorganized speech, affective flattening, alogia, anhedonia, inappropriate affect, dysphoric mood (in the form of, for example, depression, anxiety or anger), and some indications of cognitive dysfunction.

In another embodiment the present invention relates to a process for preparing a compound of formula I 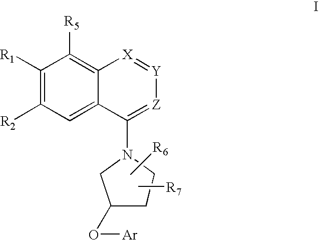
or a pharmaceutically acceptable salt, solvate or prodrug thereof,

wherein X, Y and Z are each independently CH or N with the proviso that at least one or two of X, Y and Z are N, but not all three, and with the proviso that Y and Z are not both N;

wherein R₁, R₂ and R₅ are independently H, halogen, C≡N, —COOH, —COOR₃, —CON R₃R₄, COR₃, —NR₃R₄, —NHCOR₃, —OH, (C₆-C₁₀)aryl, 5 to 7 membered heteroaryl, (C₁-C₆)alkyl, (C₂-C₆)alkenyl, (C₂-C₆)alkynyl, —O—(C₁-C₆)alkyl, —O—(C₂-C₆)alkenyl or (C₃-C₈)cycloalkyl; or, when R₁, R₂ and R₅ are independently —O—(C₁-C₆)alkyl, —O—(C₂-C₆)alkenyl, (C₁-C₆)alkyl, (C₂-C₆)alkenyl or (C₂-C₆)alkynyl, R₁ and R₂ or R₁ and R₅ may optionally be connected to form a 5 to 8 membered ring;

wherein R₃ and R₄ are independently H, (C₁-C₆)alkyl or (C₆-C₁₀)aryl said aryl optionally substituted with one or more (C₁-C₆)alkyl groups;

wherein R₆ and R₇ are each independently H, halogen, —COOR₃, —CONR₃R₄, —COR₄, NR₃R₄, —NHCOR₃, —OH, —(C₁-C₆)alkylene-OH, —HNCOOR₃, —CN, —HNCONHR₄, (C₁-C₆)alkyl, (C₂-C₆)alkoxy, C₆-C₁₀ aryl or 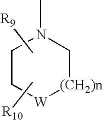
wherein n is 0 or 1;

W is carbon, oxygen or NR₈, wherein R₈ is hydrogen or (C₁-C₆)alkyl, and when W is carbon, it may be optionally substituted by halogen, —C≡N, —COOH, —COOR₃, —CONR₃R₄, —COR₃, —NR₃R₄, —NHCOR₃, —OH, (C₆-C₁₀)aryl, 5 to 7 membered heteroaryl, (C₁-C₆)alkyl, (C₁-C₆)haloalkyl (C₂-C₆)alkenyl, (C₂-C₆)alkynyl, —O—(C₁-C₆)alkyl, —O—(C₂-C₆)alkenyl or (C₃-C₈)cycloalkyl;

wherein R₉and R₁₀ are independently hydrogen or (C₁-C₈)alkyl;

or R₉ and R₁₀ may optionally combine to form a cyclic ring;

wherein Ar is phenyl, naphthyl, or a 5- to 6-membered heteroaryl ring, which heteroaryl is optionally fused to a benzo group, and which heteroaryl contains from one to four heteroatoms selected from oxygen, nitrogen and sulfur, with the proviso that said heteroaryl ring cannot contain two adjacent oxygen atoms or two adjacent sulfur atoms, and wherein each of the foregoing phenyl, naphthyl, heteroaryl, or benzo-fused heteroaryl rings may optionally be substituted with from one to three substituents independently selected from (C₁-C₈)alkyl, chloro-, bromo-, iodo, fluoro-, (C₁-C₈)hydroxyalkyl-, (C₁-C₈)alkoxy-(C₁-C₈)alkyl-, (C₃-C₈)hydroxycycloalkyl-, (C₃-C₈)cycloalkyl, (C₃-C₈)cycloalkoxy-, (C₁-C₈)alkoxy-(C₃-C₈)cycloalkyl-, (3-8 membered)heterocycloalkyl, hydroxyl(3-8 membered)heterocycloalkyl, and (C₁-C₈)alkoxy-(3-8 membered)heterocycloalkyl, wherein said alkyl, alkoxy and cycloalkyl may be optionally substituted with 1 to 3 halos and wherein each (C₃-C₈)cycloalkyl or heterocycloalkyl moiety may be independently substituted with from one to three (C₁-C₆)alkyl or benzyl groups; or

wherein Ar is a 5- to 6-membered heteroaryl ring, which heteroaryl is fused to an imidazo, pyrido, pyrimido, pyrazo, pyridazo, or pyrrolo group, and which heteroaryl contains from one to four heteroatoms selected from oxygen, nitrogen and sulfur, with the proviso that said heteroaryl ring cannot contain two adjacent oxygen atoms or two adjacent sulfur atoms, and wherein each of the foregoing fused heteroaryl rings may optionally be substituted with from one to three substituents independently selected from (C₁-C₈)alkyl, chloro-, bromo-, iodo, fluoro-, halo(C₁-C₈)alkyl, (C₁-C₈)hydroxyalkyl-, (C₁-C₈)alkoxy-(C₁-C₈)alkyl-, (C₃-C₈)hydroxycycloalkyl-, (C₃-C₈)cycloalkyl, (C₃-C₈)cycloalkoxy-, (C₁-C₈)alkoxy-(C₃-C_{8 (}3-8 membered)heterocycloalkyl, hydroxyl(3-8 membered)heterocycloalkyl, and (C₁-C₈)alkoxy-(3-8 membered)heterocycloalkyl, wherein each (C₃-C₈)cycloalkyl or heterocycloalkyl moiety may be independently substituted with from one to three (C₁-C₆)alkyl or benzyl groups; or

when Ar is phenyl, naphthyl, or heteroaryl ring, each ring may be optionally substituted with one to three substituents independently selected from (a) lactone formed from —(CH₂)ₜOH with an ortho —COOH, wherein t is one, two or three; (b) —CONR₁₄R₁₅, wherein R₁₄ and R₁₅ are independently selected from (C₁-C₈)alkyl and benzyl, or R₁₄ and R₁₅ together with the nitrogen to which they are attached form a 5- to 7-membered heteroalkyl ring that may contain from zero to three heteroatoms selected from nitrogen, sulfur and oxygen in addition to the nitrogen of the —CONR₁₄R₁₅ group, wherein when any of said heteroatoms is nitrogen it may be optionally substituted with (C₁-C₈)alkyl or benzyl, with the proviso that said ring cannot contain two adjacent oxygen atoms or two adjacent sulfur atoms; or (c) —(CH₂)ᵥNCOR₁₄R₁₅ wherein v is zero, one, two or three and —CO R₁₄R₁₅ taken together with the nitrogen to which they are attached form a 4- to 6-membered lactam ring;

comprising reacting a compound of formula III 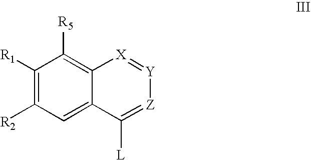
wherein X, Y and Z are as defined above;

wherein R₁, R₂ and R₅ are independently H, halogen, C≡N, —COOH, —COOR₃, —CON R₃R₄, COR₃, —NR₃R₄, —NHCOR₃, —OH, (C₆-C₁₀)aryl, 5 to 7 membered heteroaryl, (C₁-C₆)alkyl, heteroaryl, (C₂-C₆)alkenyl, (C₂-C₆)alkynyl, —O—(C₁-C₆)alkyl, —O—(C₂-C₆)alkenyl or (C₃-C₈)cycloalkyl; and, when R₁, R₂ and R₅ are independently —O—(C₁-C₆)alkyl, —O—(C₂-C₆)alkenyl, (C₁-C₆)alkyl, (C₂-C₆)alkenyl or (C₂-C₆)alkynyl, R₁ and R₂ or R₁ and R₅ may optionally be connected to form a 5 to 8 membered ring;

wherein R₃ and R₄ are independently H, (C₁-C₆)alkyl or (C₆-C₁₀)aryl said aryl optionally substituted with one or more (C₁-C₆)alkyl groups;

and L is a suitable leaving group; with a compound of formula II 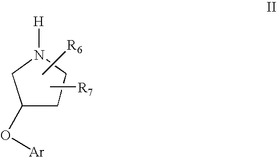
wherein Ar, R₆ and R₇ are defined above.

Examples of leaving groups include, but are not limited to chlorine, bromine, iodine, p-toluenesulfonate, C₁-C₆alkylsulfate and C₁-C₆alkanesulfonate, particularly trifluoromethanesulfonate

In a preferred embodiment, the leaving group L is chlorine.

In another aspect, the invention relates to intermediate compounds having the formula 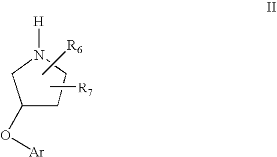
wherein R₆ and R₇ are each independently is H, halogen, —COOR₃, —CONR₃R₄, —COR₄, NR₃R₄, —NHCOR₃, —OH, —HNCOOR₃, —CN, —HNCONHR₄, (C₁-C₆)alkyl, —O(C₂-C₆)alkyl, C₆-C₁₀ aryl or 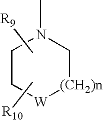
wherein n is 0 or 1;

W is carbon, oxygen or NR₈, wherein R₈ is hydrogen or (C₁-C₆)alkyl, and when W is carbon, it may be optionally substituted by halogen, —C≡N, —COOH, —COOR₃, —CONR₃R₄, —COR₃, —NR₃R₄, —NHCOR₃, —OH, (C₆-C₁₀)aryl, 5 to 7 membered heteroaryl, (C₁-C₆)alkyl, (C₁-C₆)haloalkyl (C₂-C₆)alkenyl, (C₂-C₆)alkynyl, —O—(C₁-C₆)alkyl, —O—(C₂-C₆)cycloalkyl;

wherein R₉ and R₁₀ are independently hydrogen or (C₁-C₈)alkyl;

or R₉ and R₁₀ may optionally combine to form a cyclic ring;

wherein R₃ and R₄ are independently H, (C₁-C₆)alkyl or (C₆-C₁₀)aryl said aryl optionally substituted with one or more (C₁-C₆)alkyl groups;

wherein Ar is phenyl, naphthyl, or a 5- to 6-membered heteroaryl ring, which heteroaryl is optionally fused to a benzo group, and which heteroaryl contains from one to four heteroatoms selected from oxygen, nitrogen and sulfur, with the proviso that said heteroaryl ring cannot contain two adjacent oxygen atoms or two adjacent sulfur atoms, and wherein each of the foregoing phenyl, naphthyl, heteroaryl, or benzo-fused heteroaryl rings may optionally be substituted with from one to three substituents independently selected from (C₁-C₈)alkyl, chloro-, bromo-, iodo, fluoro-, (C₁-C₈)hydroxyalkyl-, (C₁-C₈)alkoxy-(C₁-C₈)alkyl-, (C₃-C₈)hydroxycycloalkyl-, (C₃-C₈)cycloalkyl, (C₃-C₈)cycloalkoxy-, (C₁-C₈)alkoxy-(C₃-C₈)cycloalkyl-, (3-8 membered)heterocycloalkyl, hydroxyl(3-8 membered)heterocycloalkyl, and (C₁-C₈)alkoxy-(3-8 membered)heterocycloalkyl, wherein said alkyl, alkoxy and cycloalkyl may be optionally substituted with 1 to 3 halos and wherein each (C₃-C₈)cycloalkyl or heterocycloalkyl moiety may be independently substituted with from one to three (C₁-C₆)alkyl or benzyl groups; or

wherein Ar is a phenyl, naphthyl, heteroaryl or benzo-fused heteroaryl ring, each said ring may be optionally substituted with one to three substituents independently selected from phenyl, naphthyl and a 5- to 6-membered heteroaryl ring containing from one to four hetero-atoms selected from oxygen, nitrogen and sulfur, with the proviso that said heteroaryl ring cannot contain two adjacent oxygen atoms or two adjacent sulfur atoms, and wherein each independently selected phenyl, naphthyl or heteroaryl substituent may itself be substituted with from one to three (C₁-C₈)alkyl or C₃-C₈ cycloalkyl substituents, wherein examples of heteroaryl groups include, but are not limited to, pyridinyl, pyridazinyl, imidazolyl, pyrimidinyl, pyrazolyl, triazolyl, pyrazinyl, quinolyl, isoquinolyl, tetrazolyl, furyl, thienyl, isoxazolyl, thiazolyl, oxazolyl, isothiazolyl, pyrrolyl, quinolinyl, isoquinolinyl, indolyl, benzimidazolyl, benzofuranyl, cinnolinyl, indazolyl, indolizinyl, phthalazinyl, pyridazinyl, triazinyl, isoindolyl, purinyl, oxadiazolyl, thiazolyl, thiadiazolyl, furazanyl, benzofurazanyl, benzothiophenyl, benzotriazolyl, benzothiazolyl, benzoxazolyl, quinazolinyl, quinoxalinyl, naphthyridinyl, dihydroquinolyl, tetrahydroquinolyl, dihydroisoquinolyl, tetrahydroisoquinolyl, benzofuryl, furopyridinyl, pyrolopyrimidinyl, and azaindolyl;

when Ar is a phenyl, naphthyl or heteroaryl ring, each said ring may be optionally substituted with one to three substituents independently selected from (a) lactone formed from —(CH₂)ₜOH with an ortho —COOH, wherein t is one, two or three; (b) —CONR¹⁴R¹⁵, wherein R¹⁴ and R¹⁵ are independently selected from (C₁-C₈)alkyl and benzyl, or R¹⁴ and R₁₅ together with the nitrogen to which they are attached form a 5- to 7-membered heteroalkyl ring that may contain from zero to three heteroatoms selected from nitrogen, sulfur and oxygen in addition to the nitrogen of the —CONR¹⁴R¹⁵ group, wherein when any of said heteroatoms is nitrogen it may be optionally substituted with (C₁-C₈)alkyl or benzyl, with the proviso that said ring cannot contain two adjacent oxygen atoms or two adjacent sulfur atoms; or (c) —(CH₂)ᵥNCOR₁₄R₁₅ wherein v is zero, one, two or three and —COR₁₄R₁₅ taken together with the nitrogen to which they are attached form a 4- to 6-membered lactam ring.

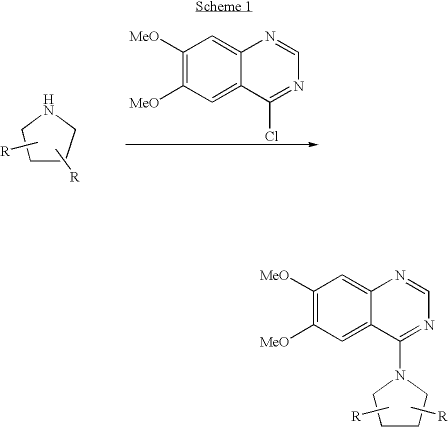

Scheme 1 depicts a coupling reaction between 4-chloro-6,7-dimethoxyquinazoline and a pyrrolidine component. This reaction is typically carried out in an inert solvent such as toluene, optionally in the presence of a base, at a temperature range from about 0° C. to about 200° C. Other suitable solvents include benzene, chloroform, dioxane, ethyl acetate, 2-propanol and xylene, and tetrahydrofuran. Alternatively, solvent mixtures such as toluene/isopropanol can be used. Preferably the reactants are heated under reflux in a solvent mixture of toluene and isopropanol for a period of from about 2 hours to about 24 hours. Another preferred set of conditions requires treating a tetrahydrofuran solution of the reactants with aqueous sodium bicarbonate at reflux. The method is not limited to 4-chloro-6,7-dimethoxyquinazoline since other substituted or unsubstituted quinazolines, cinnolines, and isoquinolines can be used provided a leaving group exists in the 4-position. A wide variety of substituted pyrrolidines may participate in this reaction.

Derivatives of 4-chloro-quinazoline are prepared by treating the corresponding 4-one compound with POCl₃ as described in General Procedure 6 below (WO 03/008388).

Methods for the synthesis of some of these pyrrolidines are described below.

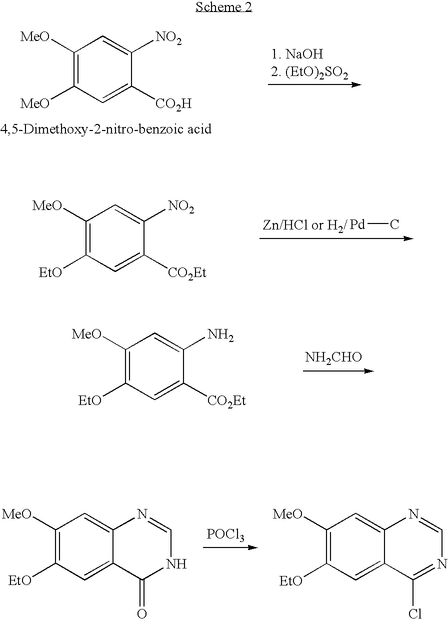

Scheme 2 shows a sequence for the synthesis of quinazoline templates in which the alkoxy groups in the 6- and 7-positions are different. According to one method, 4,5-dimethoxy-2-nitro-benzoic acid methyl ester is saponified with sodium hydroxide to give a phenolic acid. Alkylation with dialkyl sulfate or an alkyl halide provides the new substituted benzene in which the alkoxy groups are now different. Zinc reduction of the nitro group to an aniline is followed by sequential reaction with formamide and phosphorous oxychloride to provide a 4-chloroquinazoline compound possessing a methoxy group in the 7-position and a different alkoxy group in the 6-position. This quinazoline can be coupled with amines via the method described by Scheme 1.

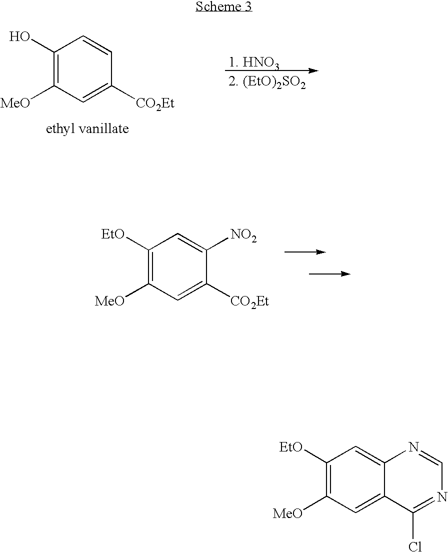

Scheme 3 shows a related method that allows for the alternative substitution pattern. In this sequence, commercially available ethyl vanillate is nitrated with nitric acid, and then alkylated with the desired electrophile. For example, diethylsulfate or iodoethane can be used to install an ethyl group as shown. Propyl sulfate would be used to install a propyl group, and so on. Zinc reduction and conversion into the 4-chloroquinazoline occurs as in Scheme 2, but the product in this case possesses a methyl group in the quinazoline 6-position, and a different alky group resides in the 7-position.

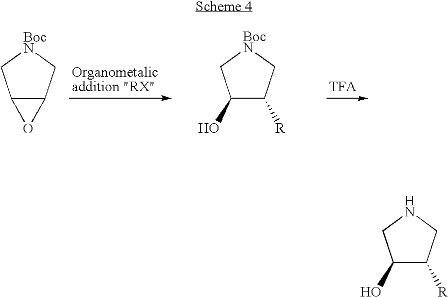

Scheme 4 describes a method for preparing N-protected pyrrolidine compounds possessing a 3-hydroxyl group and a 4-alkyl or 4-aryl group. The method begins with the epoxide shown. The epoxide can be opened with organometallic reagents such as Grignard reagents or cuprates to give the Boc protected pyrrolidine. (Hansen, S. U. and M. Bols, 1-Azaribofuranoside Analogues as designed inhibitors of purine nucleoside phosphorylase. synthesis and biological evaluation. Acta Chimica Scandinavica, 1998. 52: p. 1214-1222).

Cleavage of the Boc group with trifluoroacetic acid provides the free pyrrolidine, which can be coupled with a quinazoline ring as described in Scheme 1.

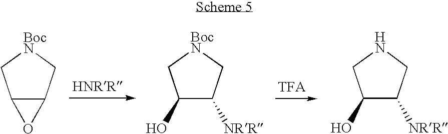

Scheme 5 depicts a method for the preparation of pyrrolidine derivatives possessing a 3-hydroxyl group and a 4-amino group. This method begins with the epoxide shown. The epoxide is opened by treatment with ammonia or a primary or secondary amine to give the Boc-protected pyrrolidine. Cleavage of the Boc group with trifluoroacetic acid provides the free pyrrolidine, which can be coupled with a quinazoline ring as described in Scheme 1.

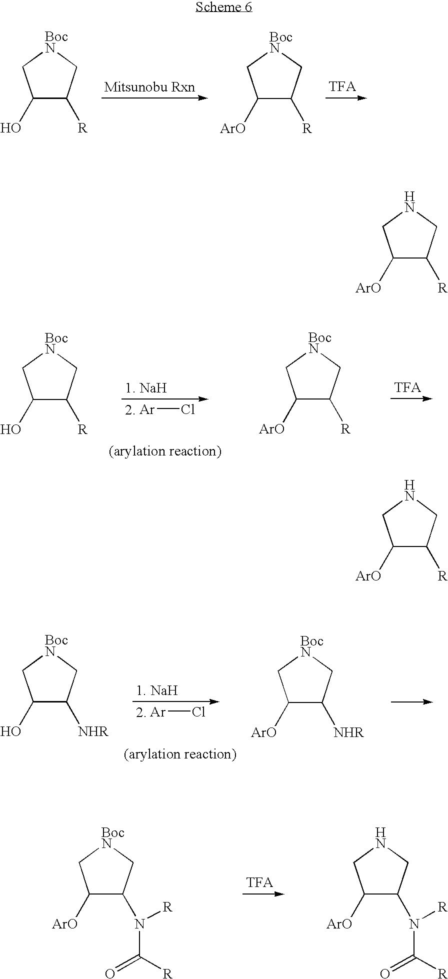

Scheme 6 shows two methods used to incorporate aryl ether groups. In one case, the well-known Mitsunobu reaction is used. (Hughes, D. L., The Mitsunobu Reaction. Organic Reactions. Vol. 42. 1992, New York, 335-656). An alternative method for incorporating the aryl ether group involves conversion of the hydroxyl group into an alkoxide by treatment with a strong base such as sodium hydride. The alkoxide is then treated with various activated aryl halides. Examples of activated aryl halides include aryl halides with heteroatoms in the 2-position of the aryl ring relative to the halide. As illustrated in the bottom of Scheme 6, when the pyrrolidine possesses an amine group, the amine may be acylated subsequent to the arylation reaction. The acylation may be performed via methods described below. In each of the methods shown in Scheme 6, Boc group deprotection with a strong acid such as trifluoroacetic acid gives the pyrrolidine derivative, which may be coupled with the desired quinazoline according to the method of Scheme 1.

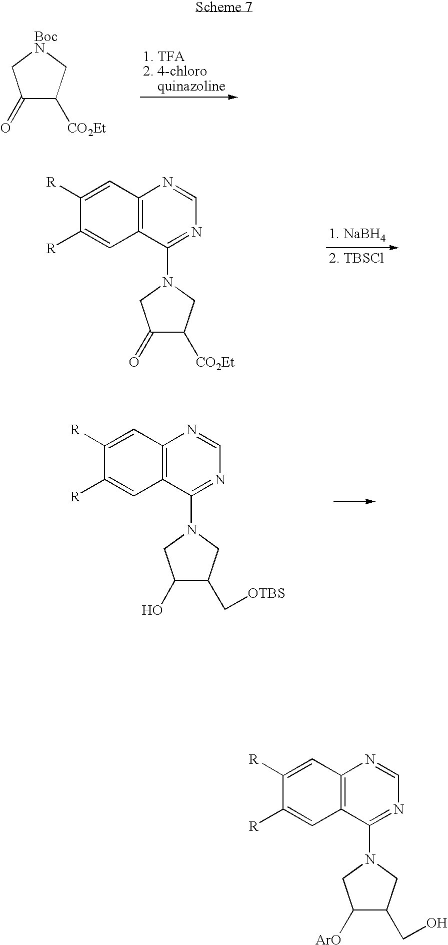

Scheme 7 depicts a method used for generating pyrrolidyl quinazoline compounds possessing a 3-aryloxy group and a 4-hydroxymethyl group on the pyrrolidine ring. This method begins with 4-oxo-pyrrolidine-1,3-dicarboxylic acid 1-tert-butyl ester 3-ethyl ester as shown. The Boc group is cleaved by treatment with TFA and the resultant pyrrolidine is coupled with the desired 4-chloroquinazoline derivative according to the Scheme 1 method. Reduction of the ketone and ester functions with sodium borohydride gives a dihydroxyl compound, which is then selectively protected on the primary hydroxyl group as a silyl ether using tert-butyldimethyl silyl chloride. Other silyl ethers could be employed as well. This is followed by conversion of the remaining free hydroxyl group into an aryl ether as described in Scheme 6. Once the aryl ether is installed, the silyl group may be cleaved under acidic conditions to provide the pyrrolidinylquinazoline possessing a 3-aryloxy group and a 4-hydroxymethyl group.

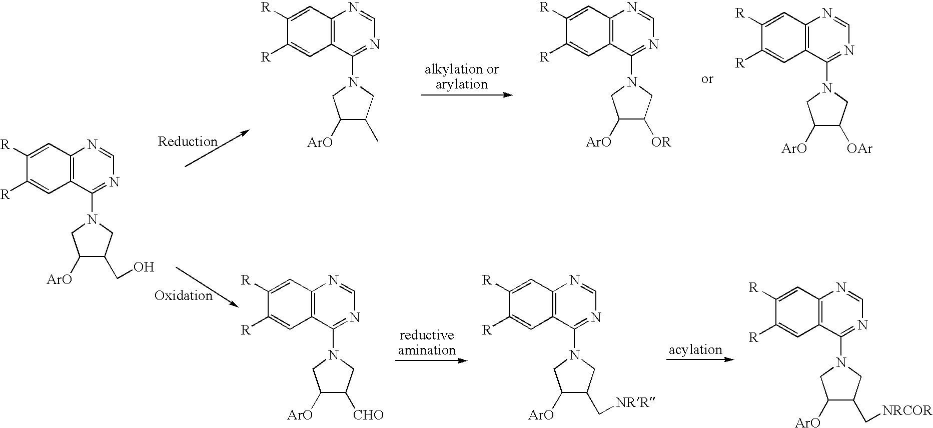

The alcohol of Scheme 7 is a versatile structure that can be used without modification or as an intermediate for the preparation of a variety of new structures. For example, as shown in Scheme 8, the hydroxyl group could be reduced to give a methyl group. One method for this reduction would require conversion of the alcohol into the methane sulfonate, and subsequent reduction with zinc in the presence of sodium iodide as described in the literature. (Fugimoto, Y. and T. Tatsuno, A novel method for reductive removal of tosyloxy and mesyloxy groups. Tetrahedron Lett., 1976. 37: p. 3325-3326). The hydroxyl group could also be alkylated or arylated to give alkyl- or aryl-ethers, respectively. In the former case this is known to those skilled in the art as the Williamson ether synthesis. (Laue, T. and A. Plagens, Named Organic Reactions. 2000, West Sussex: John Wiley & Sons). Formation of aryl ethers was described in Scheme 6. Alternatively, the alcohol could be oxidized by a variety of methods to give an aldehyde. (Tidwell, T. T., Oxidation of alcohols to carbonyl compounds via alkoxysulfonium ylides: the Moffat, Swern and related oxidations. Organic Reactions. Vol. 39. 1990, New York. 297-572). The aldehyde can then be converted into various amines via the well-known reductive amination reaction. In this reaction, the aldehyde is treated with an amine and a reducing agent in a suitable solvent. There are many effective reducing agents known to those skilled in the art. Two of the most common reducing agents are sodium cyanoborohydride and sodium triacetoxyborohydride. However, catalytic hydrogenation can also be used. Suitable solvents include various alcohols, as well as inert solvents such as methylene chloride, THF, ether, toluene, benzene, glyme, or chloroform. Preferably, alcoholic solvents are used with sodium cyanoborohydride and catalytic hydrogenation, while the inert solvents are often used with sodium triacetoxyborohydride. Primary and secondary amine products can be further converted via acylation into amides, urethanes and ureas utilizing standard acylation methods. (Bodanszky, M., Principles of Peptide Synthesis. 2nd ed. 1993, Berlin Heidelberg: Springer-Verlag), (Humphrey, J. M. and A. R. Chamberlin, Chemical Synthesis of Natural Product Peptides: Coupling Methods for the Incorporation of Noncoded Amino Acids into Peptides. Chem. Rev., 1997. 97(6): p. 2243-2266). Each of the transformations described in Scheme 8 are well known to those skilled in the art, and could be accomplished via any of a wide variety of conditions and reagents found in the literature.

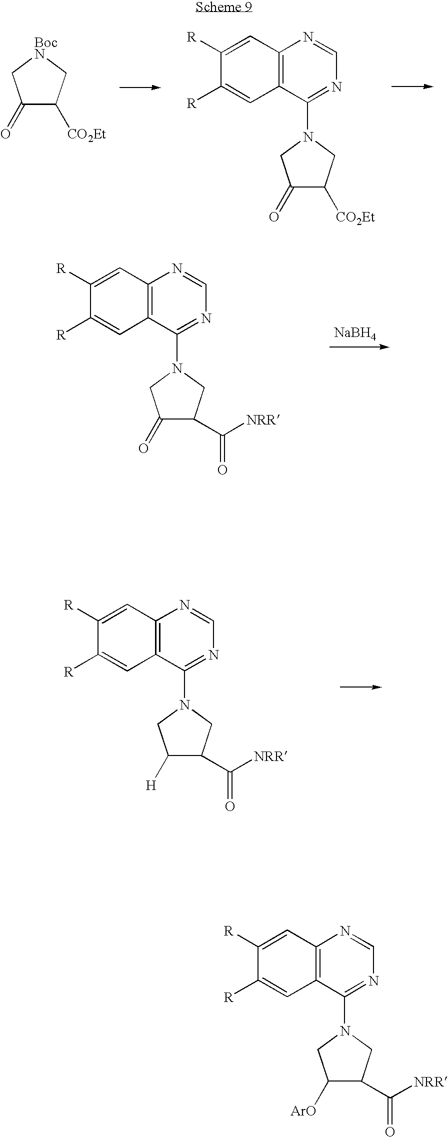

Scheme 9 shows a method useful for the preparation of pyrrolidinylquinazolines possessing aryl ether functionality in the pyrrolidine 3-position and carboxamide functionality in the pyrrolidine 4-position. The method begins with 4-oxo-pyrrolidine-1,3-dicarboxylic acid 1-tert-butyl ester 3-ethyl ester which is prepared according to the literature procedure. (Lee, J. H., et al., Synthesis and biological activity of Novel 1b-methylcarbapenems with oxyiminopyrrolidinylamide moiety. Bioorg. Med. Chem. Lett., 2003. 13: p. 4399-4403). Boc cleavage via treatment with trifluoroacetic acid precedes coupling with the desired quinazoline according to the Scheme 1 procedure. The ester functionality can then be converted into the carboxamide group via the Weinreb amination protocol. (Basha, A., M. Lipton, and S. M. Weinreb, A Mild, General Method for Conversion of Esters to Amides. Tetrahedron, 1977. 48: p. 4171-4174). An alternative method requires heating the ester and amine components in the presence of a catalytic amount of sodium cyanide. (Högberg, T. et al., Cyanide as an Efficient Catalyst in the Amminolysis of Esters. J. Org. Chem, 1987. 52: p. 2033-2036). Alternatively, the ester group could be cleaved by hydrolysis and the resultant carboxylic acid could then be coupled with amine components utilizing any of a vast array of known coupling methods. For example, this can be done via conversion of the carboxylic acid into the acid chloride under commonly known conditions. The acid chloride is then treated with the amine component in the presence of base in an inert solvent such as methylene chloride to provide the amide product. This coupling can also be mediated by specialized coupling reagents known to those skilled in the art, such as DCC, HATU, BOP-Cl, PyBrop and many others. (Humphrey, J. M. and A. R. Chamberlin, Chem. Rev., 1997. 97(6): p. 2243-2266 and Bodanszky, M., Principles of Peptide Synthesis. 2nd ed. 1993, Berlin Heidelberg: Springer-Verlag.) Suitable solvents for couplings via the acid chloride or coupling agent-mediated reactions include methylene chloride, chloroform, TCE, benzene, toluene, THF, DMF, dioxane and glyme among others. With the amide group in place, the ketone functionality would be reduced by treatment with a reducing agent such as sodium borohydride to generate an alcohol. Conversion of the alcohol into the requisite aryl ether functionality would be accomplished according to the methods of Scheme 6.

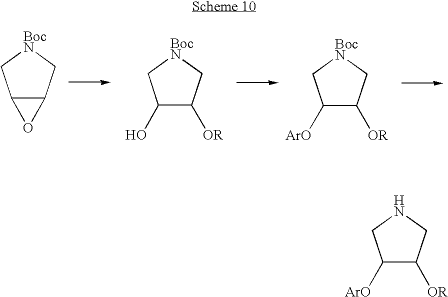

Scheme 10 shows a sequence used to prepare pyrrolidine derivatives with aryl ether substitution in the 3-position and alkyl ether substitution in the 4-position. The method shown in the scheme begins with Boc-protected 6-Oxa-3-aza-bicyclo[3.1.0]hexane, but other protecting groups may be used in place of Boc. The substrate is treated with a strong acid in an alcoholic solvent, which serves to open the epoxide to the 3-hydroxy-4-alkoxypyrrolidine. Suitable alcohols include primary alcohols such as methanol and ethanol. Suitable secondary alcohols include isopropanol. Suitable strong acids include, but are not limited to, sulfuric acid and camphorsulfonic acid. The aryl group is easily installed via the Mitsunobu reaction or via arylation of the alkoxide formed by treatment of the alcohol with sodium hydride as described herein. After deprotedtion of the protecting group the product amine can be coupled with the chloroquinazoline component as described in Scheme 1. Alternatively, the deprotection step and coupling to the quinazoline can be accomplished prior to incorporation of the aryl ether substituent. According to this alternative, Boc cleavage with trifluoroaectic acid is used to form the free pyrrolidine and the quinazoline is then introduced, as is Scheme 1. Treatment with sodium hydride or another strong base is used to generate an alkoxide, which is then arylated as described in Scheme 6.

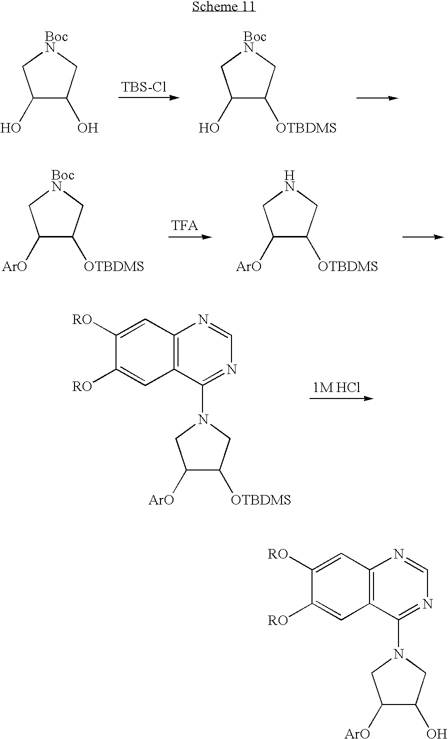

Scheme 11 describes a method effective for the preparation of pyrrolidyl-quinazoline compounds in which the pyrrolidine component possesses a 3-aryloxy substituent and a 4-hydroxyl group. The method begins with 3,4-dihydroxy-pyrrolidine-1-carboxylic acid tert-butyl ester, which is prepared according to a published procedure. (Donohoe, T. J., et al., Flexibility in the Partial Reduction of 2,5-Disubstituted Pyrroles: Application to the Synthesis of DMDP. Org. Let., 2003. 5(7): p. 999-1002). The diol can be mono-protected on one of the alcohol functions by treatment with a protecting group such as tert-butyl-dimethylsilyl chloride. The nature of the protecting group is not critical, as other protecting groups are likely to work as well. After protection, the remaining hydroxyl group can be arylated via the Mitsunobu reaction or by arylation of the corresponding alkoxide in an aromatic nucleophilic substitution reaction. These methods are described herein. With the aryl group installed, the next step is to cleave the N-protecting group without cleaving the silyl group. In the illustrated case, this is accomplished via treatment with TFA. The resultant amine is then coupled with the desired 4-chloroquinazoline derivative as described in Scheme 1, and the silyl group is finally cleaved with an acid such as aqueous HCl

The compound of the invention may be administered either alone or in combination with pharmaceutically acceptable carriers, in either single or multiple doses. Suitable pharmaceutical carriers include inert solid diluents or fillers, sterile aqueous solutions and various organic solvents. The pharmaceutical compositions formed thereby can then be readily administered in a variety of dosage forms such as tablets, powders, lozenges, liquid preparations, syrups, injectable solutions and the like. These pharmaceutical compositions can optionally contain additional ingredients such as flavorings, binders, excipients and the like. Thus, the compound of the invention may be formulated for oral, buccal, intranasal, parenteral (e.g. intravenous, intramuscular or subcutaneous), transdermal (e.g. patch) or rectal administration, or in a form suitable for administration by inhalation or insulation.

For oral administration, the pharmaceutical compositions may take the form of, for example, tablets or capsules prepared by conventional means with pharmaceutically acceptable excipients such as binding agents (e.g. pregelatinized maize starch, polyvinylpyrrolidone or hydroxypropyl methylcellulose); fillers (e.g. lactose, microcrystalline cellulose or calcium phosphate); lubricants (e.g. magnesium stearate, talc or silica); disintegrants (e.g. potato starch or sodium starch glycolate); or wetting agents (e.g. sodium lauryl sulphate). The tablets may be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (e.g. sorbitol syrup, methyl cellulose or hydrogenated edible fats); emulsifying agents (e.g. lecithin or acacia); non-aqueous vehicles (e.g. almond oil, oily esters or ethyl alcohol); and preservatives (e.g. methyl or propyl p-hydroxybenzoates or sorbic acid).

For buccal administration, the composition may take the form of tablets or lozenges formulated in conventional manner.

The compounds of the invention may be formulated for parenteral administration by injection, including using conventional catheterization techniques or infusion. Formulations for injection may be presented in unit dosage form, e.g. in ampules or in multi-dose containers, with an added preservative. They may take such forms as suspensions, solutions or emulsions in oily or aqueous vehicles, and may contain formulating agents such as suspending, stabilizing and/or dispersing agents. Alternatively, the active ingredient may be in powder form for reconstitution with a suitable vehicle, e.g. sterile pyrogen-free water, before use.

When a product solution is required, it can be made by dissolving the isolated inclusion complex in water (or other aqueous medium) in an amount sufficient to generate a solution of the required strength for oral or parenteral administration to patients. The compounds may be formulated for fast dispersing dosage forms (fddf), which are designed to release the active ingredient in the oral cavity. These have often been formulated using rapidly soluble gelatin-based matrices. These dosage forms are well known and can be used to deliver a wide range of drugs. Most fast dispersing dosage forms utilize gelatin as a carrier or structure-forming agent. Typically, gelatin is used to give sufficient strength to the dosage form to prevent breakage during removal from packaging, but once placed in the mouth, the gelatin allows immediate dissolution of the dosage form. Alternatively, various starches are used to the same effect.

The compounds of the invention may also be formulated in rectal compositions such as suppositories or retention enemas, e.g. containing conventional suppository bases such as cocoa butter or other glycerides.

For intranasal administration or administration by inhalation, the compound of the invention is conveniently delivered in the form of a solution or suspension from a pump spray container that is squeezed or pumped by the patient or as an aerosol spray presentation from a pressurized container or a nebulizer, with the use of a suitable propellant, e.g. dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. The pressurized container or nebulizer may contain a solution or suspension of the active compound. Capsules and cartridges (made e.g. from gelatin) for use in an inhaler or insufflator may be formulated containing a powder mix of a compound of the invention and a suitable powder base such as lactose or starch.

Aerosol formulations for treatment of the conditions referred to above (e.g. migraine) in the average adult human are preferably arranged so that each metered dose or “puff” of aerosol contains about 20 mg to about 1000 mg of the compound of the invention. The overall daily dose with an aerosol will be within the range of about 100 mg to about 10 mg. Administration may be several times daily, e.g. 2, 3, 4 or 8 times, giving for example, 1, 2 or 3 doses each time.

A proposed daily dose of the compound of the invention for oral, parenteral, rectal or buccal administration to the average adult human for the treatment of the conditions referred to above is from about 0.01 mg to about 2000 mg, preferably from about 0.1 mg to about 200 mg of the active ingredient of formula I per unit dose which could be administered, for example, 1 to 4 times per day.

Assay methods are available to screen a substance for inhibition of cyclic nucleotide hydrolysis by the PDE 10 and the PDEs from other gene families. The cyclic nucleotide substrate concentration used in the assay is ⅓ of the Kₘ concentration, allowing for comparisons of IC₅₀ values across the different enzymes. PDE activity is measured using a Scintillation Proximity Assay (SPA)-based method as previously described (Fawcett et al., 2000). The effect of PDE inhibitors is determined by assaying a fixed amount of enzyme (PDEs 1-11) in the presence of varying substance concentrations and low substrate, such that the IC50 approximates the Kᵢ(cGMP or cAMP in a 3:1 ratio unlabelled to [³H]-labeled at a concentration of ⅓ Km). ). The final assay volume is made up to 100 μl with assay buffer [20 mM Tris-HCl pH 7.4, 5 mM MgCl₂, 1 mg/ml bovine serum albumin]. Reactions are initiated with enzyme, incubated for 30-60 min at 30° C. to give <30% substrate turnover and terminated with 50 μl yttrium silicate SPA beads (Amersham) (containing 3 mM of the respective unlabelled cyclic nucleotide for PDEs 9 and 11). Plates are re-sealed and shaken for 20 min, after which the beads were allowed to settle for 30 minutes in the dark and then counted on a TopCount plate reader (Packard, Meriden, Conn.). Radioactivity units can be converted to percent activity of an uninhibited control (100%), plotted against inhibitor concentration and inhibitor IC₅₀ values can be obtained using the “Fit Curve’ Microsoft Excel extension.

Using such assay, compounds of the present invention were determined to have an IC₅₀ activity for inhibiting PDE10 activity of less than about 10 micromolar.

The following Examples illustrate the present invention. It is to be understood, however, that the invention, as fully described herein and as recited in the claims, is not intended to be limited by the details of the following Examples.

### EXAMPLES

Experimental Procedures

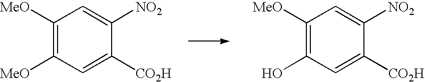

Preparation 1. 5-Hydroxy-4-methoxy-2-nitro-benzoic acid. To 4,5-dimethoxy-2-nitro-benzoic acid was added 6 M HCl (60 mL). The resultant yellow mixture was heated to 100° C. for 3 h, and then cooled to rt. The resultant solid was dissolved in 100 mL of water and poured into a slurry of 9 M HCl and crushed ice. The mixture was extracted twice with ethyl acetate and the combined extracts were washed with brine, dried over magnesium sulfate, and concentrated to give 14.7 g of a pale yellow solid. Recrystallization from ethyl acetate/hexanes provided 10.8 g (79%) of the title compound.

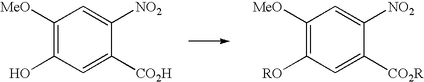

General Procedure 1. 4-Methoxy-2-nitro-5-alkoxy-benzoic acid alkyl ester. In General Procedures 1-6, R is C₁ to C₆ alkyl. 5-Hydroxy-4-methoxy-2-nitro-benzoic acid (250 mg, 1.18 mmol) in DMF (2.0 mL) was added potassium carbonate (250 mg, 2.36 mmol)and 2.1 molar equivalents of the desired dialkylsulfate. The mixture is stirred at 85° C. for 8 h, cooled to rt, diluted with water, and extracted twice with ethyl acetate. The extracts are washed sequentially with 1 N NaOH and brine, dried with magnesium sulfate, and concentrated to give the title compound.

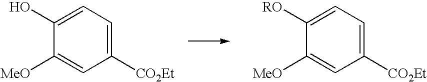

General Procedure 2. 4-Alkoxy-3-methoxy-benzoic acid ethyl ester. To ethyl vanillate and an excess of potassium carbonate in DMF is added the disired 1.2 molar equivalents of the desired dialkylsulfate. The mixture is stirred for 24 h at room temperature, and then diluted with water and extracted with ether. The combined extracts are washed with brine, dried, and concentrated to give the title compound.

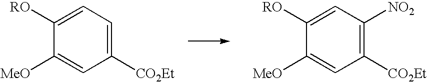

General Procedure 3. 4-Alkoxy-5-methoxy-2-nitro-benzoic acid ethyl ester. To the desired 4-alkoxy-3-methoxy-benzoic acid ethyl ester (ca. 10.0 g) in 12 mL of sulfuric acid at 0° C. is added dropwise 8 mL of a 1:1 mixture of sulfuric and nitric acids at such a rate to maintain the reaction temperature below 15° C. The mixture is then stirred at rt for 1 h and poured into 100 g of cruched ice. The resultant aqueous mixture is extracted 3× with ethyl acetate and the combined extracts is washed with brine, dried with magnesium sulfate and concentrated. Silica gel chromatography eluting with hexane/ethyl acetate provides the title compound as a yellow solid.

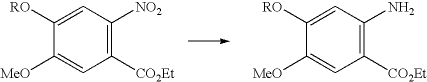

General Procedure 4. 2-Amino-4-alkoxy-5-methoxy-benzoic acid ethyl ester. To a slurry of the desired 4-alkoxy-5-methoxy-2-nitro-benzoic acid ethyl ester in 6M HCl in an ice bath is added in portions an excess of zinc powder while maintaning the reaction temperature below 25° C. When TLC analysis indicates full consumption of starting material the mixture is diluted with cold water and extracted 3× with chloroform. The combined extracts are washed with brine and concentrated to provide the title compound as a white solid.

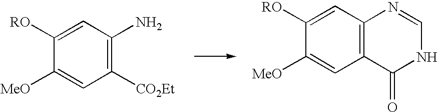

General Procedure 5. 7-Alkoxy-6-methoxy-3H-quinazolin-4-one. To the desired 2-amino-4-alkoxy-5-methoxy-benzoic acid ethyl ester in formamide is added an excess of equivallents of ammonium carbonate. The mixture is heated to 170° C for 24 h, and then cooled to rt and poured into 25 ml. of water. The resultant precipitate is filtered to give a yellow solid. Silica gel chromatography eluting with hexane/ethyl acetate provides the title compound.

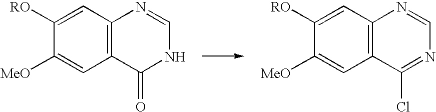

General Procedure 6. 4-Chloro-7-alkoxy-6-methoxy-quinazoline. A sample of 7-ethoxy-6-methoxy-3H-quinazolin-4-one in POCl₃ is refluxed for 2 h. and then poured into a mixture of saturated NaHCO₃ and ethyl acetate. The mixture is stirred 15 min and the layers are separated. The organic portion is washed with brine, dried with magnesium sulfate and concentrated. Silica gel chromatography eluting with 5:1 hexanes/ethyl acetate provides the title compound.

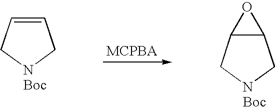

Preparation 2. 6-Oxa-3-aza-bicyclo[3.1.0]hexane-3-carboxylic acid tert-butyl ester. A mixture of N-Boc-2,5-dihydro-pyrrole (21.2 g, 125.4 mmol) and 70% MCPBA (54.1 g, 314 mmol) in toluene (500 mL) was stirred at rt for 24 h, after which the suspension was filtered and concentrated. The residue was dissolved in ether, and washed sequentially with 10% sodium hydrogen sulfite, 1 M NaOH, and brine. The solution was then dried with magnesium sulfate, filtered and concentrated. The residue was purified by silica gel chromatography eluting with 85:15 hexanes/ethy lacetate, to provide 8.85 g (38%) of the title compound as a colorless oil.

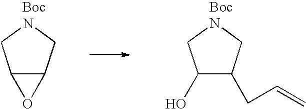

Preparation 3. 3-Allyl-4-hydroxy-pyrrolidine-1-carboxylic acid tert-butyl ester. 6-Oxa-3-aza-bicyclo[3.1.0]hexane-3-carboxylic acid tert-butyl ester (277 mg, 15 mmol) in ether at 0° C. was added 3 molar equivalents of allyl magnesium bromide, dropwise. The mixture was warmed to rt until complete by TLC analysis, and was then quenched with saturated ammmonium chloride and extracted with ethyl acetate. Silica gel chromatography eluting with 5:1 hexane/ethyl acetate to give 141 mg (41%) of the title compound as a colorless oil.

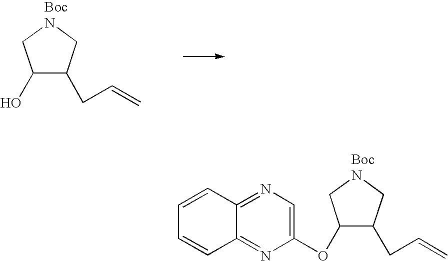

Preparation 4.3-Allyl-4-(quinoxalin-2-yloxy)-pyrrolidine-1-carboxylic acid tert-butyl ester. To 3-allyl-4-hydroxy-pyrrolidine-1-carboxylic acid tert-butyl ester (114 mg, 0.5 mmol) in THF was added 1.15 molar equivallents of triphenylphosphine, 1.2 molar equivallents of DEAD, and 1.4 molar equivallents of 2-quinoxalol. The solution was stirred at rt for 4 h, and was then concentrated to a slurry. The slurry was dissolved in chloroform and washed sequentially with 1 N sodium hydroxide, 10% citric acid, and brine. The organic solution was concentrated and the residue chromatographed on silica gel eluting with 5:1 hexane/ethyl acetate to give 96 mg (54%) of the title compound as a colorless oil.

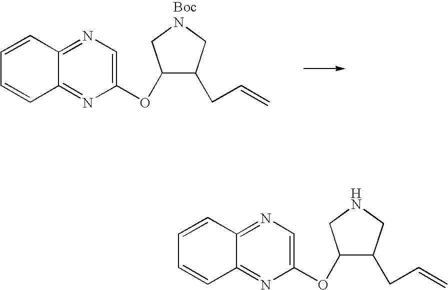

Preparation 5. 2-(4-Allyl-pyrrolidin-3-yloxy)-quinoxaline. To 3-allyl-4-(quinoxalin-2-yloxy)-pyrrolidine-1-carboxylic acid tert-butyl ester in methylene chloride was added an equal volume of trifluoroacetic acetic acid. The solution was stirred until complete by TLC and was then concentrated to give the title compound as a colorless oil.

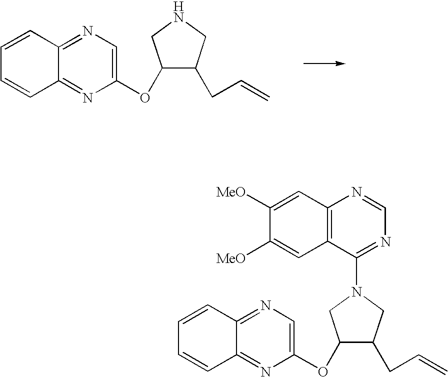

### Example 1

4-[3-Allyl-4-(quinoxalin-2-yloxy)-pyrrolidin-1-yl[-6,7-dimethoxy-quinazoline. To 2-(4-allyl-pyrrolidin-3-yloxy)-quinoxaline 53 mg (0.2 mmol) in 2 mL of THF and 0.5 mL of water was added 3 molar equivalents of potassium bicarbonate and 1.1 molar equivallents of 6,7-dimethoxy-4-chloroquinoxaline. The mixture was heated at reflux for 24 h and then was concentrated under vacuum. The residue was chromatographed eluting with 2:98 ethanol/ethyl acetate to yield 29 mg (29%) of the title compound as a colorless oil. Mass spectrum (M+H) m/z=444.3.

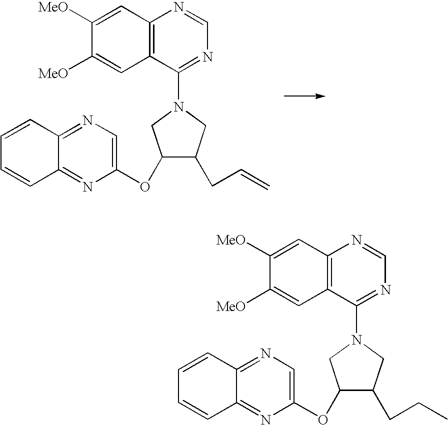

### Example 2

6,7-Dimethoxy-4-[3-propyl-4-(quinoxalin-2-yloxy)-pyrrolidin-1-yl]-quinazoline. To 23 mg (0.05 mmol) of 4-[3-allyl-4-(quinoxalin-2-yloxy)-pyrrolidin-1-yl]-6,7-dimethoxy-quinazoline was added 50 mg of ammonium formate followed by a slurry of 5 mg of 10% palladium on carbon in a small amount of ethanol. The mixture was heated to 75° C. for 1 h, and cooled to rt. The mixture was filtered through celite and concentrated. The resultant oil was dissolved in chloroform, washed sequentially with water and brine, and chromatographed through silica gel eluting with 5:95 ethanol/ethyl acetate to yield a colorless oil. The oil was treated with ethereal HCl and concentrated to provide 19 mg (79%) of the title compound as a white solid. Mass spectrum (M+H) m/z=446.3.

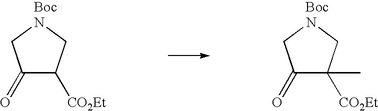

Preparation 6. 3-Methyl-4-oxo-pyrrolidine-1.3-dicarboxylic acid 1-tert-butyl ester 3-ethyl ester. To 4-oxo-pyrrolidine-1,3-dicarboxylic acid 1-tert-butyl ester 3-ethyl ester (1.02 g, 4.0 mmol) in THF at rt was added DBU (4.1 mmol). After 15 min, iodomethane (4.0 mmol) was added and the resultant mixture was stirred for 1 h. The mixture was concentrated and the resultant slurry was partitioned between chloroform and water. The aqueous portion was extracted twice and the combined organic solutions were dried over magnesium sulfate, filtered and concentrated. Silica gel chromatography (4:1 hexanes/ethyl acetate) provided 677 mg (63%) of the title compound as a colorless oil.

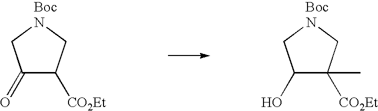

Preparation 7. 4-Hydroxy-3-methyl-pyrrolidine-1,3-dicarboxylic acid 1-tert-butyl ester 3-ethyl ester. To 3-methyl-4-oxo-pyrrolidine-1,3-dicarboxylic acid 1-tert-butyl ester 3-ethyl ester (371 mg, 1.0 mmol) in ethanol was added sodium borohydride (19 mg, 5.0 mmol), and the resultant mixture was stirred at rt for 1 h. The mixture was diluted with water and extracted with chloroform. The extracts were dried via filtration through cotton and concentrated to give 223 mg (82%) of the title compound as a colorless oil.

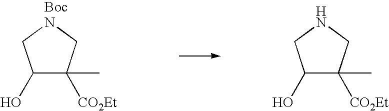

Preparation 8. 4-Hydroxy-3-methyl-pyrrolidine-3-carboxylic acid ethyl ester. Prepared similarly to preparation 5.

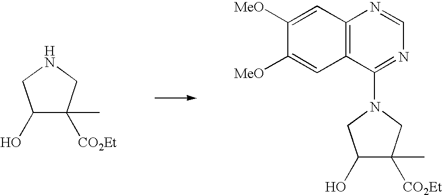

Preparation 9. 1-(6.7-Dimethoxy-quinazolin-4-yl)-4-hydroxy-3-methyl-pyrrolidine-3-carboxylic acid ethyl ester. Prepared similarly to Example 1.

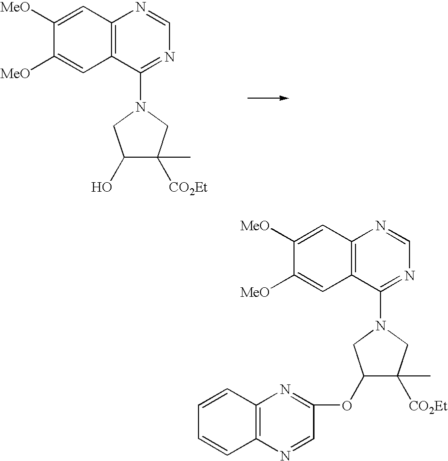

### Example 3

1-(6,7-Dimethoxy-quinazolin-4-yl)-3-methyl-4-(quinoxalin-2-yloxy)-pyrrolidine-3-carboxylic acid ethyl ester. To 1-(6,7-dimethoxy-quinazolin-4-yl)-4-hydroxy-3-methyl-pyrrolidine-3-carboxylic acid ethyl ester (37 mg, 0.10 mmol) in THF was added 5 mg (0.12 mmol) of sodium hydride. The mixture was stirred for 30 min, and then 2-chloroquinoxaline was added. The mixture was stirred at rt for 24 h, and then was quenched with water and extracted with chloroform. Silica gel chromatography eluting with 2% ethanol in ethyl acetate provided a colorless oil. Treatment of the oil with HCl in ether and drying under vacuum provided the title compound as it's hydrochloride salt in the amount of 41 mg (83%). Mass spectrum (M+H) m/z=490.4.

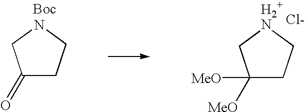

Preparation 10. 3,3-Dimethoxy-pyrrolidine. To 3-oxo-pyrrolidine-1-carboxylic acid tert-butyl ester (4.05 g) in 20 mL of trimethylorthoformate was added 40 mL of a 4 N solution of HCl in methanol. The solution was stirred for 4.5 h, and the volume was reduced to ca 15 mL under vacuum. The solution was diluted with 60 mL of ethyl acetate and the mixture was stirred for 4 h. The resultant solids were collected via filtration to provide 2.97 g of the title compound as an off-white solid.

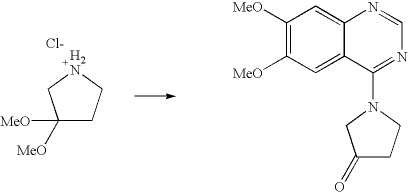

Preparation 11. 1-(6,7-Dimethoxy-quinazolin-4-yl)-pyrrolidin-3-one. A mixture of 3,3-dimethoxy-pyrrolidine (2.97 g, 17.8 mmol) and 4-chloro-6,7-dimethoxyquinazoline (4.38 g, 19.5 mmol) in THF (30 mL) and satd. NaHCO₃ (50 mL) was heated to reflux for 5 h. The mixture was cooled and partitioned between water and ethyl acetate. The extract was washed with brine, and then extracted twice with 1 N HCl. After 20 min the combined acidic extracts were poured carefully into an excess of potassium carbonate in water. The mixture was then extracted three times with methylene chloride and the combined extracts were concentrated to a slurry. The slurry was diluted with an equal volume of hexanes and stirred over night. The resultant solids were collected via filtration and rinsed with ether to provide 3.71 g (76%) of the title compound as a tan powder. MS=274.2.

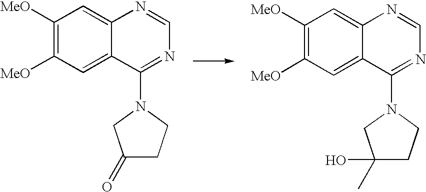

Preparation 12. 1-(6,7-Dimethoxy-quinazolin-4-yl)-3-methyl-pyrrolidin-3-ol. To 1-(6,7-dimethoxy-quinazolin-4-yl)-pyrrolidin-3-one (78 mg, 0.286 mmol) in THF (8 mL) at 0° C. was added a solution of methyl magnesium bromide (3 M in THF; 143 uL, 0.43 mmol). The solution was stirred for 10 min, and then quenched with ammonium chloride. The mixture was then partitioned between water and methylene chloride. The organic portion was dried and concentrated. Purification via silica gel chromatography (MeOH/CH₂Cl₂) provided 38 mg (46%) of the title compound as a tan solid.

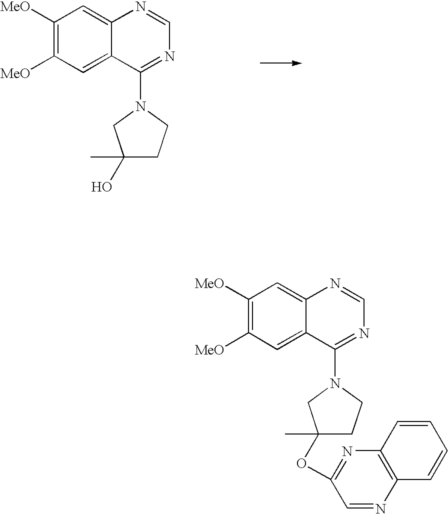

### Example 4

6,7-Dimethoxy-4-[3-methyl-3-(quinoxalin-2-yloxy)-pyrrolidin-1-yl]-quinazoline. Prepared similarly to Example 1.

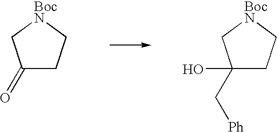

Preparation 13. 3-Benzyl-3-hydroxy-pyrrolidine-1-carboxylic acid tert-butyl ester. To 3-oxo-pyrrolidine-1-carboxylic acid tert-butyl ester (1.50 g, 8.11 mmol) in tetrahydrofuran (24 mL) in an ice bath was added a solution of 2.0 M benzyl magnesium chloride in tetrahydrofuran (12.2 mmol, 6.1 mL). The mixture was stirred for 1 h, and then was warmed to rt and stirred for 16 h. The mixture was then concentrated and partitioned between ether and water. The pH was adjusted to ca. 2 with 1 M HCl. The organic portion was dried, filtered and concentrated. Silica gel chromatography eluting with 4:1 hexanes/ethyl acetate provided 700 mg (19%) of the title compound as a pale yellow oil. 1H NMR (400 MHz, CDCl₃) δ 1.40 (s, 9H), 1.68-1.78 (m, 1H), 1.79-1.88 (m, 1H), 2.39-2.45 (d, J=23.2 Hz, 1H), 2.86 (s, 2 H), 3.21-3.36 (m, 2H), 3.38-3.45 (m, 2H).

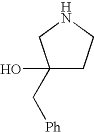

Preparation 14. 3-Benzyl-pyrrolidin-3-ol. Prepared similarly to Preparation 5. 1H NMR (400 MHz, CD₃OD, HCl salt) 1.90-1.92 (m, 1H), 1.99-2.07 (m, 1H), 2.99 (s, 2H), 3.04-3.13 (m, 2H), 3.36-3.43 (m, 2H).

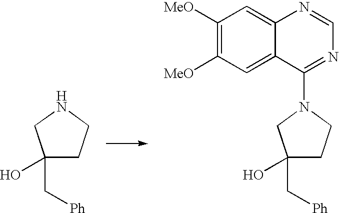

### Example 5

3-Benzyl-1-(6,7-dimethoxy-quinazolin-4-yl)-pyrrolidin-3-ol. Prepared similarly to Example 1. MS: (M⁺H m/z=366.1)

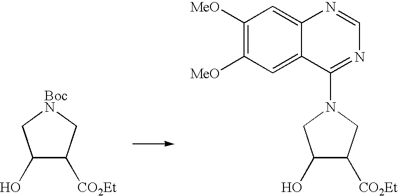

Preparation 15. 1-(6,7-Dimethoxy-quinazolin-4-yl)-4-hydroxy-pyrrolidine-3-carboxylic acid ethyl ester. To 4-hydroxy-pyrrolidine-1,3-dicarboxylic acid 1-tert-butyl ester 3-ethyl ester (2.25 g, 259 mmol) in methylene chloride (20 mL) was added TFA (6 mL). The solution was stirred for 1 h, and was then diluted with chloroform (25 mL). The mixture was concentrated, and to the residue was added saturated NaHCO₃ (40 mL), tetrahydrofuran (15 mL), and one molar equivallent of 4-chloro-6,7-dimethoxyquinazoline. The mixture was heated to 60° C. and stirred overnight. The mixture was cooled, diluted with water, and extracted 3× with 2-butanol. The mixture was purified by stirring as a slurry in ethyl acetate for 2 h. The solid was collected via filtration to yield 869 mg (29%) of the title compound as a white powder.

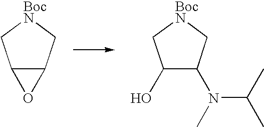

Preparation 16. 3-Hydroxy-4-(isopropyl-methyl-amino)-pyrrolidine-1-carboxylic acid tert-butyl ester. This compound was prepared similarly to the published protocol [EP0485952A2]. [M+H] m/z=259.3.

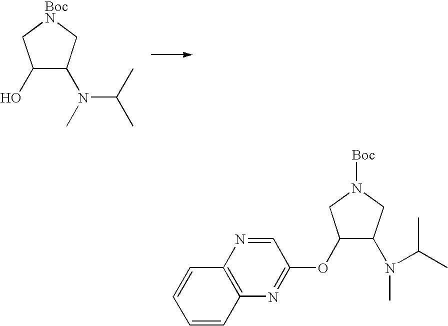

Preparation 17. 3-(Isopropyl-methyl-amino)-4-(quinoxalin-2-yloxy)-pyrrolidine-1-carboxylic acid tert-butyl ester. Prepared similarly to Preparation 4.

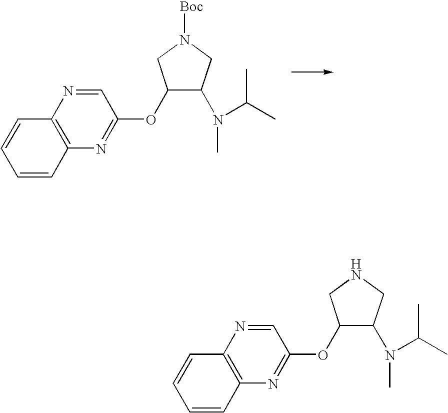

Preparation 18. Isopropyl-methyl-[4-(quinoxalin-2-yloxy)-pyrrolidin-3-yl-1-amine. Prepared similarly to Preparation 5. 13C NMR (CDCl₃) 17.6, 18.0, 32.7, 51.3, 51.5, 53.7, 69.1, 80.9, 126.9, 127.6,129.1, 130.4, 139.5, 140.0, 140.5.

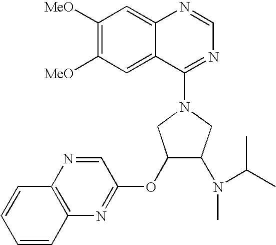

### Example 6

[1-(6,7-Dimethoxy-quinazolin-4-yl)-4-(quinoxalin-2-yloxy)-pyrrolidin-3-yl]-isopropyl-methyl-amine. Prepared similarly to Example 1. MS (M+H) m/z=475.4.

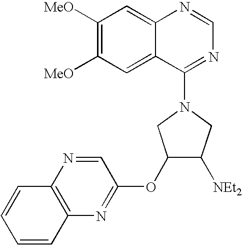

The following Examples 7-15 are prepared similarly to Example 1.

### Example 7

[1-(6,7-Dimethoxy-quinazolin-4-yl)-4-(quinoxalin-2-yloxy)-Pyrrolidin-3-yl]-diethyl-amine. Prepared similarly. MS (M+H) m/z=447.2.

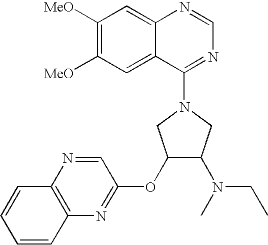

### Example 8

[1-(6,7-Dimethoxy-quinazolin-4-yl)4-(quinoxalin-2-yloxy)-pyrrolidin-3-yl]-ethyl-methyl-amine. Prepared similarly. MS (M+H) m/z=461.4.

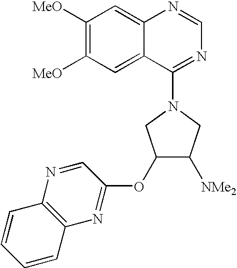

### Example 9

[1-(6,7-Dimethoxy-quinazolin-4-yl)-4-(quinoxalin-2-yloxy)-pyrrolidin-3-yl]-dimethyl-amine. Prepared similarly. MS (M+H) m/z=419.2

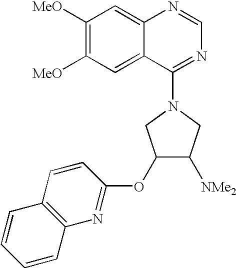

### Example 10

[1-(6,7-Dimethoxy-quinazolin-4-yl)-4-(quinolin-2-yloxy)-pyrrolidin-3-yl]-dimethyl-amine. Prepared similarly. MS: (M⁺H) m/z=446.4.

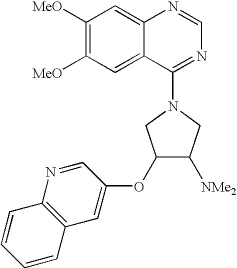

### Example 11

[1-(6,7-Dimethoxy-quinazolin-4-yl)-4-(quinolin-3-yloxy)-pyrrolidin-3-yl]-dimethyl-amine. Prepared similarly. MS: (M⁺H) m/z=446.4.

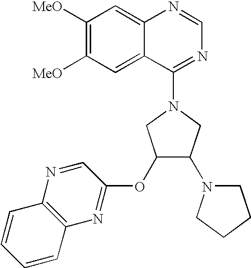

### Example 12

6,7-Dimethoxy-4-[4′-(quinoxalin-2-yloxy)-[1,3′]bipyrrolidinyl-1′-yl]-quinazoline. Prepared similarly. MS (M+H) m/z 473.4.

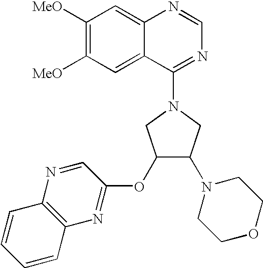

### Example 13

6,7-Dimethoxy-4-[3-morpholin-4-yl-4-(quinoxalin-2-yloxy)-pyrrolidin-1-yl]-quinazoline. Prepared similarly. MS (M+H) m/z 489.4.

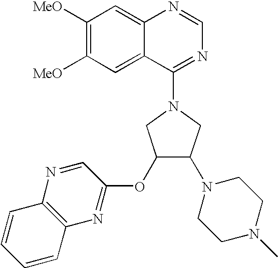

### Example 14

6,7-Dimethoxy-4-[3-(4-methyl-piperazin-1-yl)-4-(quinoxalin-2-yloxy)-pyrrolidin-1-yl]-quinazoline. Prepared similarly. Mass spectrum (M⁺H) m/z=502.4.

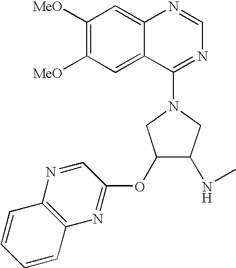

### Example 15

Prepared similarly. [1-(6,7-Dimethoxy-quinazolin-4-yl)-4-(quinoxalin-2-yloxy)-pyrrolidin-3-yl]-methyl-amine. MS (M+H) m/z=345.3.

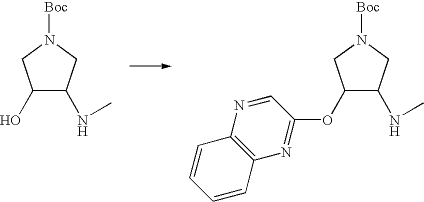

Preparation 19. 3-Methylamino-4-(quinoxalin-2-yloxy)-pyrrolidine-1-carboxylic acid tert-butyl ester. Prepared similarly to Preparation 4.

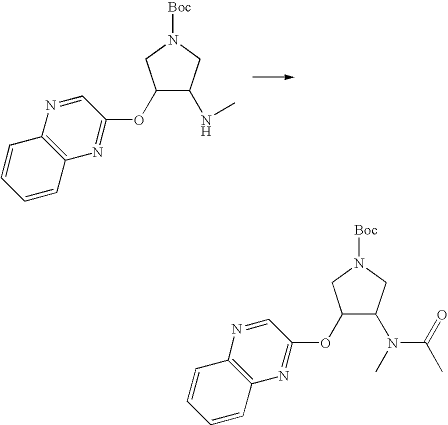

Preparation 20. 3-(Acetyl-methyl-amino)-4-(quinoxalin-2-yloxy)-pyrrolidine-1-carboxylic acid tert-butyl ester. To 3-methylamino-4-(quinoxalin-2-yloxy)-pyrrolidine-1-carboxylic acid tert-butyl ester (240 mg, 0.70 mmol) and triethylamine (78 mg, 767 mmol) in 7 mL of methylene chloride in an ice bath was added acetic anhydride (75 mg, 0.73 mmol) as a solution in 3 mL of methylene chloride. The solution was stirred at ice bath temperature for 1.5 h, and at rt for 24 h. The mixture was washed with 0.1 M KHSO₄, dried and concentrated to give 291 mg of the title compound as a pale yellow foam.

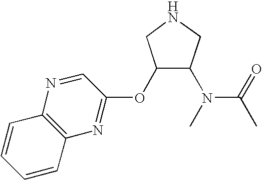

Preparation 21. N-Methyl-N-[4-(quinoxalin-2-yloxy)-pyrrolidin-3-yl]-acetamide. Prepared similarly to Preparation 5. MS (M+H) m/z=287.3.

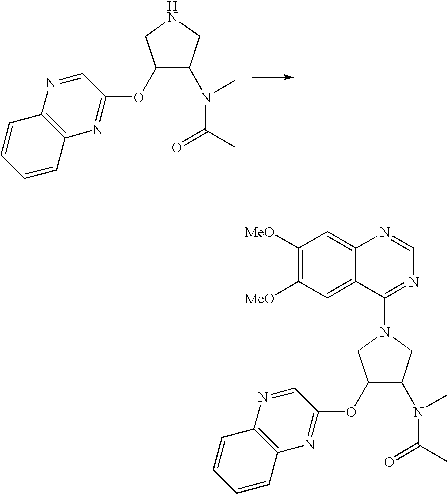

### Example 16

N-[1-(6,7-Dimethoxy-quinazolin-4-yl)-4-(quinoxalin-2-yloxy)-pyrrolidin-3-yl]-N-methyl-acetamide. Prepared similarly to Example 1. MS (M+H) m/z=475.4.

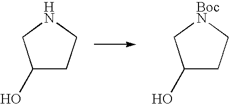

Preparation 22. 3-Hydroxy-pyrrolidine-1-carboxylic acid tert-butyl ester. To 3-hydroxypyrrolidine (10.0 g, 114.8 mmol) and triethylamine (12.8 g, 17.6 mL) in methylene chloride (225 mL) at 0° C. was added dropwise over 30 min di-tert-butyldicarbonate (25.3 g, 115.9 mmol). The mixture was warmed to rt and stirred for 16 h. The organic material was then washed with saturated ammonium chloride, dried via passage through a cotton plug, and concentrated. The residue was chromatographed through silic gel eluting with ca. 1:1 hexanes/ethyl acetate to provide the title compound in the amount of 20.4 g (95%). 1H NMR (500 MHz, CDCl₃) 1.42 (s, 9H), 1.80-1.98 (m, 2H), 2.20-2.27 (d, J=2.3 Hz, 1H), 3.27-3.30 (m, 1H); 3.32-3.50 (m, 3H), 4.39-4.43 (m, 1H).

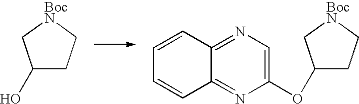

Preparation 23. 3-(Quinoxalin-2-yloxy)-pyrrolidine-1-carboxylic acid tert-butyl ester. A mixture of 3-hydroxy-pyrrolidine-1-carboxylic acid tert-butyl ester (10.1 g, 54 mmol), 2-quinoxalinol (11.8 g, 81 mmol), triphenylphosphine (17.0 g, 64.8 mmol), and diethylazodicarboxylate (DEAD; 11.3 g, 64.8 mmol) was stirred in tetrahydrofuran (250 mL) for 2 h. The mixture was then concentrated and the residue dissolved in methylene chloride. The organic solution was washed sequentially with 1 N NaOH and 5% citric acid, and then was dried through cotton anc concentrated. Silica gel chromatography eluting with hexanes/ethylacetate (3:1) provided 15.4 g (91%) of the title compound as a colorless semi-solid. MS (M+H) m/z=316.2.

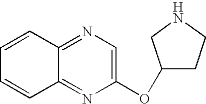

Prepararation 24. 2-(Pyrrolidin-3-yloxy)-quinoxaline. Prepared similarly to Preparation 5.

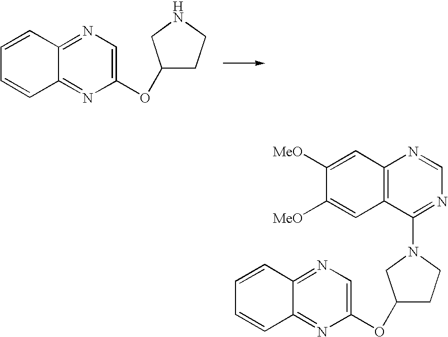

### Example 17

6,7-Dimethoxy-4-[3-(quinoxalin-2-yloxy)-pyrrolidin-1-yl]-quinazoline. Prepared similarly to Example 1. A mixture of 2-(pyrrrolidin-3-yloxy)-quinoxaline (10.5 g, 48.9 mmol), 6,7-dimethoxy-4-chloroquinazoline (11.0 g, 48.9 mmol), and potassium carbonate (33.8 g, 244 mmol) in THF (500 mL)and water (100 mL) was heated to 75° C. for 16 h. The mixture was concentrated under vacuum and the residue was dissolved in methylene chloride. The solution was washed with 1N NaOH and dried through a cotton plug. The solution was then concentrated and the resultant brown foam was chromatographed on silica gel eluting with 9:1 ethyl acetate/methanol to provide 17.3 g of an off-white powder. Trituration with ethyl acetate/methanol gave 15.0 g of an off-white powder. The solid was suspended in isopropanol and treated with 1.0 molar equivallents of sulfuric acid. After stirring overnight, the solids were collected via filtration to give 18.0 g of the title compound as a white powder. Mass spectrum (M+H) m/z=404.2.

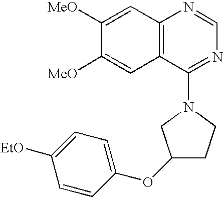

The following Examples 18-40 are prepared similarly to Example 17.

### Example 18

4-[3-(4-Ethoxy-phenoxy)-pyrrolidin-1-yl]-6,7-dimethoxy-quinazoline. MS (M+H) m/z=396.2.

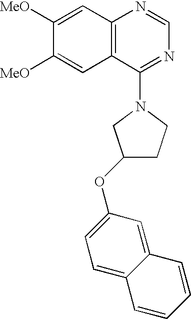

### Example 19

6,7-Dimethoxy-4-[3-(naphthalen-2-yloxy)-pyrrolidin-1-yl]-quinazoline. Mass spectrum (M+H) m/z=402.2.

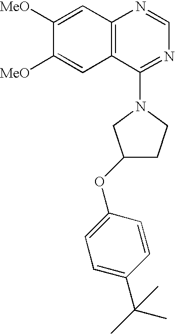

### Example 20

4-[3-(4-tert-Butyl-phenoxy)-pyrrolidin-1-yl]-6,7-dimethoxy-quinazoline. Mass spectrum (M+H) m/z=408.3.

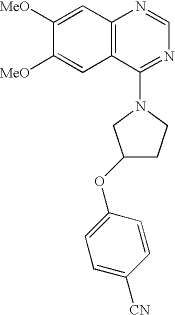

### Example 21

4-[1-(6,7-Dimethoxy-quinazolin-4-yl)-pyrrolidin-3-yloxy]-benzonitrile. Mass spectrum (M+H) m/z=377.1.

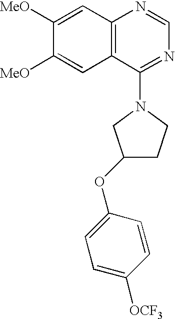

### Example 22

6,7-Dimethoxy-4-[3-(4-trifluoromethoxy-phenoxy)-pyrrolidin-1-yl]-quinazoline. Mass spectrum (M+H) m/z=436.2.

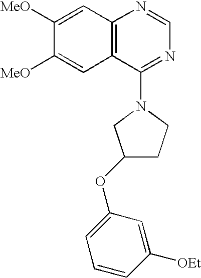

### Example 23

4-[3-(3-Ethoxy-phenoxy)-pyrrolidin-1-yl]-6,7-dimethoxy-quinazoline. Mass spectrum (M+H) m/z=396.2.

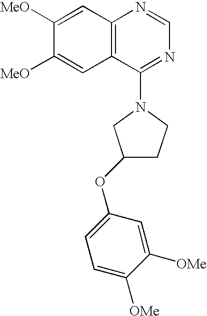

### Example 24

4-[3-(3,4-Dimethoxy-phenoxy)-pyrrolidin-1-yl]-6,7-dimethoxy-quinazoline. Mass spectrum (M+H)=412.2.

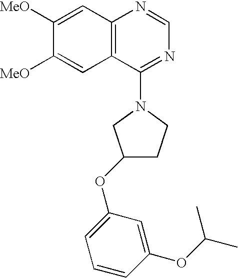

### Example 25

4-[3-(3-Isopropoxy-phenoxy)-pyrrolidin-1-yl]-6,7-dimethoxy-quinazoline. Mass spectrum (M+H) m/z=394.2.

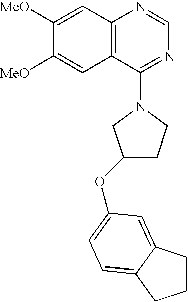

### Example 26

4-[3-(Indan-5-yloxy)-pyrrolidin-1-yl]-6,7-dimethoxy-quinazoline. Mass spectrum (M+H) m/z=392.2.

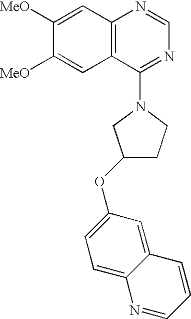

### Example 27

6,7-Dimethoxy-4-[3-(quinolin-6-yloxy)-pyrrolidin-1-yl]-quinazoline. Mass spectrum (M+H) m/z=403.2.

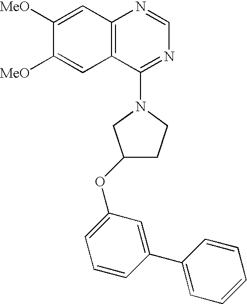

### Example 28

4-[3-(Biphenyl-3-yloxy)-pyrrolidin-1-yl]-6,7-dimethoxy-quinazoline. Mass spectrum (M+H) m/z=428.2.

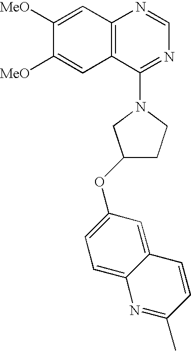

### Example 29

6,7-Dimethoxy-4-[3-(2-methyl-quinolin-6-yloxy)-pyrrolidin-1-yl]-quinazoline. Mass spectrum (M+H) m/z=417.2.

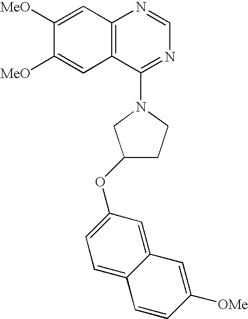

### Example 30

6,7-Dimethoxy-4-[3-(7-methoxy-naphthalen-2-yloxy)-pyrrolidin-1-yl]-quinazoline. Mass spectrum (M+H) m/z=432.2.

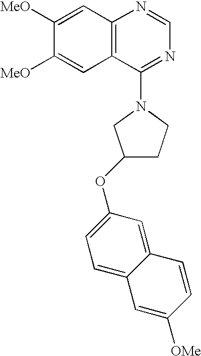

### Example 31

6,7-Dimethoxy-4-[3-(6-methoxy-naphthalen-2-yloxy)-pyrrolidin-1-yl]-quinazoline. Mass spectrum (M+H) m/z=432.2.

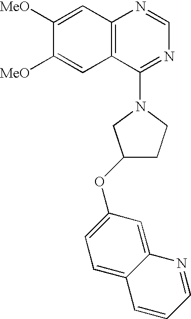

### Example 32

6,7-Dimethoxy-4-[3-(quinolin-7-yloxy)-pyrrolidin-1-yl]-quinazoline. Mass spectrum (M+H) m/z=403.2.

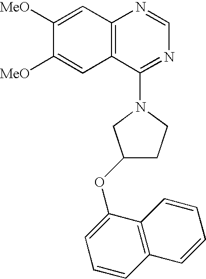

### Example 33

6,7-Dimethoxy-4-[3-(naphthalen-1-yloxy)-pyrrolidin-1-yl]-quinazoline Mass spectrum (M+H) m/z=402.2.

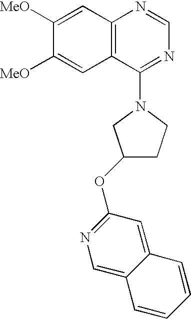

### Example 34

4-[3-(Isoquinolin-3-yloxy)-pyrrolidin-1-yl]-6,7-dimethoxy-quinazoline. Mass spectrum (M+H) m/z=403.2.

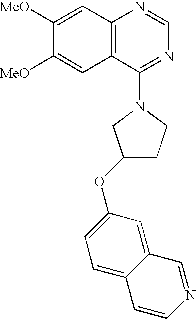

### Example 35

4-[3-(Isoquinolin-7-yloxy)-pyrrolidin-1-yl]-6,7-dimethoxy-quinazoline. Mass spectrum (M+H) m/z=403.2.

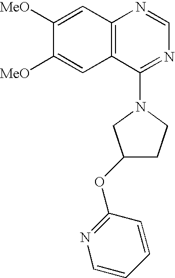

### Example 36

6,7-Dimethoxy-4-[3-(pyridin-2-yloxy)-pyrrolidin-1-yl]-quinazoline. Mass spectrum (M+H) m/z=353.1.

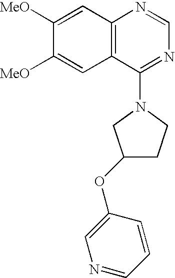

### Example 37

6,7-Dimethoxy-4-[3-(pyridin-3-yloxy)-pyrrolidin-1-yl]-quinazoline. Mass spectrum (M+H) m/z=353.1.

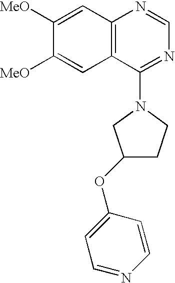

### Example 38

6,7-Dimethoxy-4-[3-(pyridin-4-yloxy)-pyrrolidin-1-yl]-quinazoline. Mass spectrum (M+H) m/z=353.2.

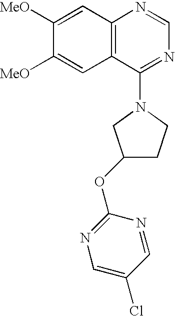

### Example 39

4-[3-(5-Chloro-pyrimidin-2-yloxy)-pyrrolidin-1-yl]-6,7-dimethoxy-quinazoline. Mass spectrum (M+H) m/z=388.1.

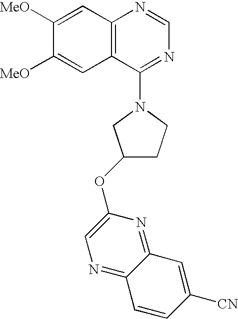

### Example 40

3-[1-(6,7-Dimethoxy-quinazolin-4-yl)-pyrrolidin-3-yloxy]-quinoxaline-6-carbonitrile. Mass spectrum (M⁺H) m/z=429.2.

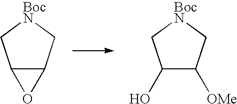

Preparation 25. 3-Hydroxy-4-methoxy-pyrrolidine-1-carboxylic acid tert-butyl ester. To 6-oxa-3-aza-bicyclo[3.1.0]hexane-3-carboxylic acid tert-butyl ester (505 mg, 2.73 mmol) in methanol (25 mL) was added camphorsulfonic acid (317 mg, 1.36 mmol). After stirring over night the mixture was partitioned between methylene chloride and water. The organic portion was dried, concentrated and chromatographed eluting with 4:6 ethyl acetate/hexanes to provide the title compound.

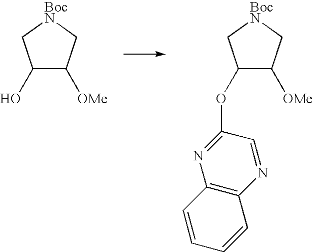

Preparation 26. 3-Methoxy-4-(quinoxalin-2-yloxy)-pyrrolidine-1-carboxylic acid tert-butyl ester. To 3-hydroxy-4-methoxy-pyrrolidine-1-carboxylic acid tert-butyl ester (235 mg, 1.08 mmol) and 2-chloroquinoxaline (213 mg, 1.29 mmol) in tetrahydrofuran (5 mL) was added a 60% dispersion of sodium hydride in mineral oil (51 mg, 1.29 mmol). The mixture was stirred for 3 h, and then was partitioned between methylene chloride and water. The organic portion was dried, concentrated and chromatographed. The product fractions were pooled and concentrated.

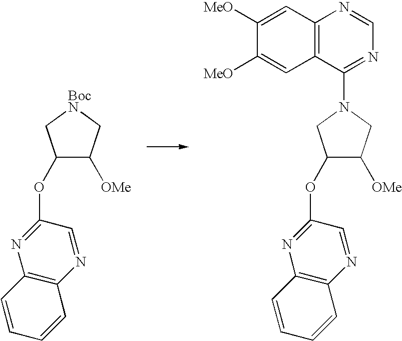

### Example 41

6,7-Dimethoxy-4-[3-methoxy-4-(quinoxalin-2-yloxy)-pyrrolidin-1-yl]-quinazoline. To 3-methoxy-4-(quinoxalin-2-yloxy)-pyrrolidine-1-carboxylic acid tert-butyl ester (373 mg, 1.0 mmol) was added methylene chloride (5 mL) and trifluoroacetic acid (5 mL). After 10 min, the solution was concentrated under vacuum, and re-concentrated again from 5 mL of chloroform. The residue was dissolved in water and washed with ether. The aqueous solution was then basified with 1 N NaOH and extracted twice with methylene chloride. The extracts were dried and concentrated. The residue was dissolved in tetrahydrofuran (5 mL) and saturated sodium bicarbonate was added (5 mL) followd by 4-chloro-6,7-dimethoxyquinazoline (1.08 mmol, 242 mg). The resultant mixture was heated to 60° C. for 5 h. The mixture was cooled to rt and partitioned between methylene chloride and water. The organic portion was dried via passaage through a cotton plug and concentrated. The residue was treated with 4 M methanolic HCl (0.75 mL, 3 mmol). The volume was then reduced by 50% under vacuum and the mixture was stirred for 1 h. The white solids were collected via filtration to provide 312 mg (75%) of the title compound as it's hydrochloride salt. Mass Spectrum (M+H) m/z=434.4.

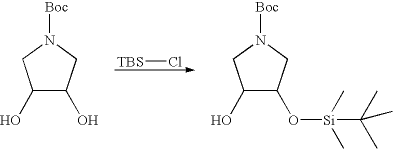

Preparation 27. 3-(tert-Butyl-dimethyl-silanyloxy)-4-hydroxy-pyrrolidine-1-carboxylic acid tert-butyl ester. To 3,4-dihydroxy-pyrrolidine-1-carboxylic acid tert-butyl ester (0.50 g, 2.46 mmol) in tetrahydrofuran at rt was added a 60% dispersion of sodium hydride in mineral oil (108 mg, 2.71 mmol). The mixture was stirred for 20 min. Tert-butyidimethylsilyl chloride was then added and the resultant mixture was stirred at rt for 16 h. The mixture was then concentrated under vacuum and the residue was dissolved in methylene chloride. The organic solution was washed with 10% citric acid, dried through cotton, and concentrated to provide 670 mg of the title compound as a colorless oil.

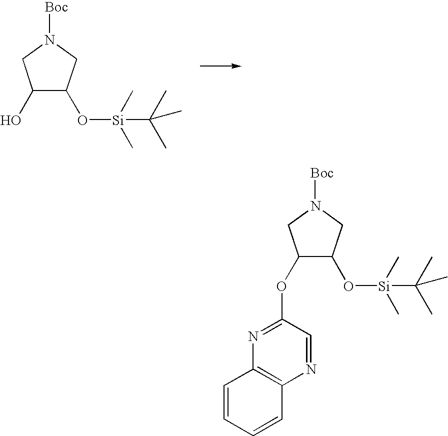

Preparation 28. 3-(tert-Butyl-dimethyl-silanyloxy)-4-(quinoxalin-2-yloxy)-pyrrolidine-1-carboxylic acid tert-butyl ester. A mixture of 3-(tert-butyl-dimethyl-silanyloxy)-4-hydroxy-pyrrolidine-1-carboxylic acid tert-butyl ester (0.67 g, 2.11 mmol), 2-quinoxalinol (0.46 g, 3.17 mmol), triphenylphosphine (0.66 g, 2.54 mmol), and diethylazodicarboxylate was stirred in tetrahydrofuran (20 mL) at 70° C. for 1 h. The mixture was then concentrated and the residue dissolved in methylene chloride. The solution was then washed twice with 1 M NaOH, once with 5% citric acid, dried through cotton, and concentrated. Silica gel chromatography eluting with hexanes/ethyl acetate (9:1) then provided 745 mg (79%) of the title compound as a colorless semi-solid.

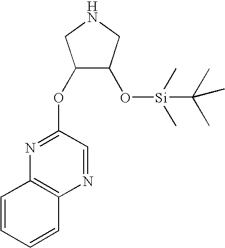

Preparation 29. 2-[4-(tert-Butyl-dimethyl-silanyloxy)-pyrrolidin-3-yloxy]-quinoxaline. This compound was Prepared similarly to Preparation 5.

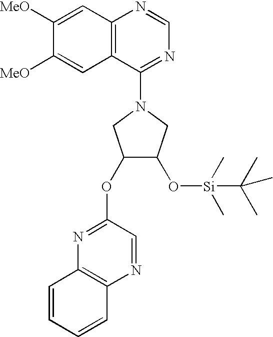

Preparation 30. 4-[3-(tert-Butyl-dimethyl-silanyloxy)-4-(quinoxalin-2-yloxy)-pyrrolidin-1-yl]-6,7-dimethoxy-quinazoline. This compound was prepared similarly to Example 1.

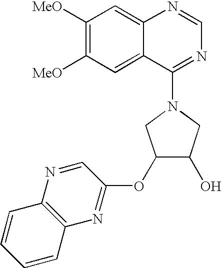

### Example 42

1-(6,7-Dimethoxy-quinazolin-4-yl)-4-(quinoxalin-2-yloxy)-pyrrolidin-3-ol. A solution of 4-[3-(tert-butyl-dimethyl-silanyloxy)-4-(quinoxalin-2-yloxy)-pyrrolidin-1-yl]-6,7-dimethoxy-quinazoline in 25 mL of methanol and 10 mL of 1 M HCl was stirred at 60° C. for 1.5 h. The solution was then concentrated and the solids were stirred as a slurry in water and isopropanol for 16 h. The solids were then collected by filtration and dried under vacuum to provide 480 mg of the hydrochloride salt of title compound as a white powder. Mass spectrum (M+H) m/z=420.2.

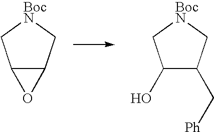

Preparation 31 3-Benzyl-4-hydroxy-pyrrolidine-1-carboxylic acid tert-butyl ester. To 6-oxa-3-aza-bicyclo[3.1.0]hexane-3-carboxylic acid tert-butyl ester (1.32 g, 7.16 mmol) was added a 19% solution of benzyl magnesium bromide in tetrahydrofuran (22.1 g, 21.5 mmol). The mixture was stirred for 20 h, and was then poured into 0.5 M KHSO₄. The mixture was extracted three times with methylene chloride and the combined extracts were dried through cotton and concentrated. Silica gel chromatography eluting with hexanes/ethyl acetate (1:1) provided 459 mg (24%) of the title compound as a tan oil.

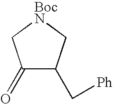

Preparation 32. 3-Benzyl-4-oxo-pyrrolidine-1-carboxylic acid tert-butyl ester. To 3-benzyl-4-hydroxy-pyrrolidine-1-carboxylic acid tert-butyl ester (297 mg, 1.07 mmol) and 4 angstrom molecular sieves in methylene chloride (20 mL) was added pyridinium chlorochromate. The mixture was stirred at rt for 16 h, and was then filtered through Celite and concentrated. Silica gel chromatography eluting with 1:1 hexanes/ethyl acetate gave 160 mg (54%) of the title compound as a pale yellow oil.

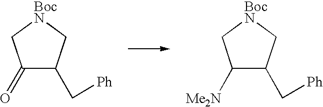

Preparation 33. 3-Benzyl-4-dimethylamino-pyrrolidine-1-carboxylic acid tert-butyl ester. A solution comprised of 2.0 M dimethylamine in tetrahydrofuran (1.48 mL, 2.96 mmol), acetic acid (231 mg, 3.85 mmol), sodium triacetoxy borohydride ( 377 mg, 1.78 mmol), and 3-benzyl-4-oxo-pyrrolidine-1-carboxylic acid tert-butyl ester (163 mg, 0.59 mmol) was stirred at rt for 24 h. The mixture was diluted with 1 M NaOH and stirred for 1.5 h. The mixture was then extracted three times with methylene chloride and the combined extracts were then dried and concentrated. Silica gel chromatography eluting with 3:1 ethyl acetate/hexanes provided 92 mg of the title compound as a colorless oil.

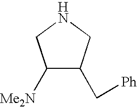

Preparation 34. (4-Benzyl-pyrrolidin-3-yl)-dimethyl-amine. Prepared similarly to Preparation 5.

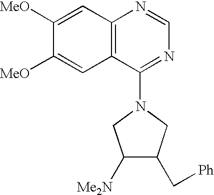

### Example 43

[4-Benzyl-1-(6,7-dimethoxy-quinazolin-4-yl)-pyrrolidin-3-yl]-dimethyl-amine. Prepared similarly to Example 1. Mass spectrum (M+H) m/z=393.4.

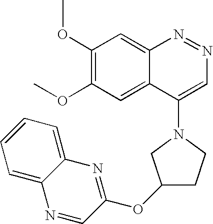

### Example 44

6,7-Dimethoxy-4-[3-(quinoxalin-2-yloxy)-pyrrolidin-1-yl]-cinnoline. Prepared similarly to Example 1, substituting 4-chloro-6,7-dimethoxy-cinnoline for 4-chloro-6,7-dimethoxy-quinazoline. MS: (M+H) m/z=404.3.

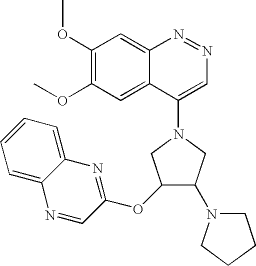

### Example 45

6,7-Dimethoxy-4-[4′-(quinoxalin-2-yloxy)-[1,3′]bipyrrolidinyl-1′-yl]-cinnoline. Prepared similarly to Example 1, substituting 4-chloro-6,7-dimethoxy-cinnoline for 4-chloro-6,7-dimethoxy-quinazoline. MS (M+H) m/z=473.1.

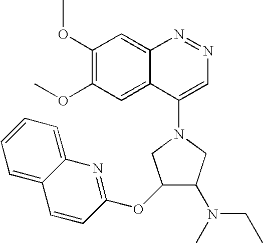

### Example 46

[1-(6,7-Dimethoxy-cinnolin-4-yl)-4-(quinolin-2-yloxy)-pyrrolidin-3-yl]-ethyl-methyl-amine. Prepared similarly to Example 1, substituting 4-chloro-6,7-dimethoxy-cinnoline for 4-chloro-6,7-dimethoxy-quinazoline. MS: (M+H) m/z=460.4.

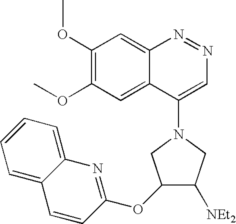

### Example 47

[1-(6,7-Dimethoxy-cinnolin-4-yl)-4-(quinolin-2-yloxy)-pyrrolidin-3-yl]-diethyl-amine. Prepared similarly to Example 1, substituting 4-chloro-6,7-dimethoxy-cinnoline for 4-chloro-6,7-dimethoxy-quinazoline. MS (M⁺H) m/z=474.4.

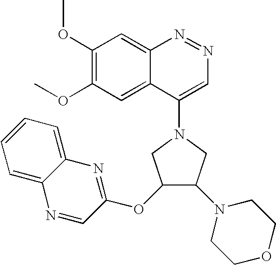

### Example 48

6,7-Dimethoxy-4-[3-morpholin-4-yl-4-(quinoxalin-2-yloxy)-pyrrolidin-1-yl]-cinnoline. Prepared similarly to Example 1, substituting 4-chloro-6,7-dimethoxy-cinnoline for 4-chloro-6,7-dimethoxy-quinazoline. MS (M+H) m/z=489.4.

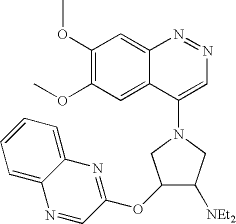

### Example 49

[1-(6,7-Dimethoxy-cinnolin-4-yl)-4-(quinoxalin-2-yloxy)-pyrrolidin-3-yl]-diethyl-amine Prepared similarly to Example 1, substituting 4-chloro-6,7-dimethoxy-cinnoline for 4-chloro-6,7-dimethoxy-quinazoline. MS (M+H) m/z=475.4.

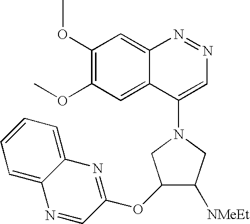

### Example 50

[1-(6,7-Dimethoxy-cinnolin-4-yl)-4-(quinoxalin-2-yloxy)-pyrrolidin-3-yl]-ethyl-methyl-amine. Prepared similarly to Example 1, substituting 4-chloro-6,7-dimethoxy-cinnoline for 4-chloro-6,7-dimethoxy-quinazoline. MS (M+H) m/z=461.4.

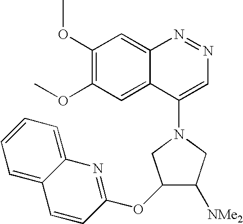

### Example 51

[1-(6,7-Dimethoxy-cinnolin-4-yl)-4-(quinolin-2-yloxy)-pyrrolidin-3-yl]-dimethyl-amine Prepared similarly to Example 1, substituting 4-chloro-6,7-dimethoxy-cinnoline for 4-chloro-6,7-dimethoxy-quinazoline. MS (M+H) m/z=446.4.

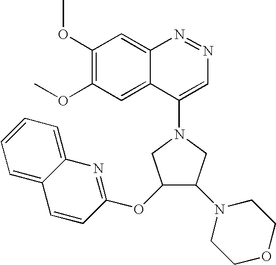

### Example 52

6,7-Dimethoxy-4-[3-morpholin-4-yl-4-(quinolin-2-yloxy)-pyrrolidin-1-yl]-cinnoline. Prepared similarly to Example 1, substituting 4-chloro-6,7-dimethoxy-cinnoline for 4-chloro-6,7-dimethoxy-quinazoline. MS (M+H) m/z=488.4.

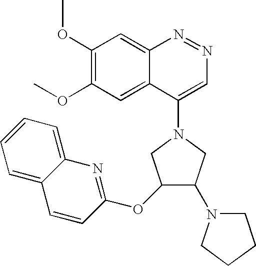

### Example 53

6,7-Dimethoxy-4-[4′-(quinolin-2-yloxy)-[1,3′]bipyrrolidinyl-1′-yl]-cinnoline. Prepared similarly to Example 1, substituting 4-chloro-6,7-dimethoxy-cinnoline for 4-chloro-6,7-dimethoxy-quinazoline. MS (M+H) m/z=472.4.

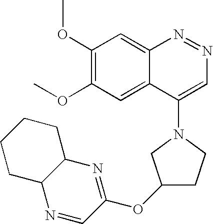

### Example 54

4-[3-(4a,5,6,7,8,8a-Hexahydro-quinoxalin-2-yloxy)-pyrrolidin-1-yl]-6,7-dimethoxy-quinazoline. Prepared similarly to Example 1. MS (M+H) m/z=408.2.

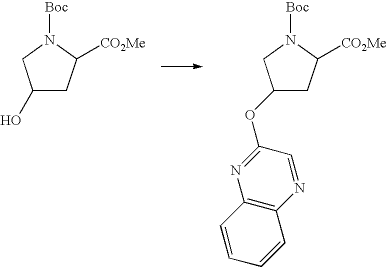

Preparation 35. 4-(Quinoxalin-2-yloxy)-pyrrolidine-1,2-dicarboxylic acid 1-tert-butyl ester 2-methyl ester. A mixture of 4-hydroxy-pyrrolidine-1,2-dicarboxylic acid 1-tert-butyl ester 2-methyl ester (5.0 g, 20.4 mmol), 2-quinoxalinol (4.47 g, 30.6 mmol), triphenylphosphine (6.42 g, 24.5 mmol), and diethylazodicarboxylate (4.26 g, 24.5 mmol) in THF (100 mL) was stirred at 70° C. for 4 h. The mixture was cooled, dissolved in methylene chloride, washed twice with 1 N NaOH and once with 5% citric acid, dried and concentrated. Silica gel chromatography eluting with hexanes/ethylacetate (3:1) provided 7.09 g (93%) of the title compound as an amorphous solid. MS (M+H)=374.1.

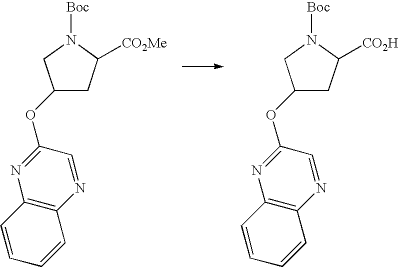

Preparation 36. 4-(Quinoxalin-2-yloxy)-pyrrolidine-1,2-dicarboxylic acid 1-tert-butyl ester. To 4-(quinoxalin-2-yloxy)-pyrrolidine-1,2-dicarboxylic acid 1-tert-butyl ester 2-methyl ester (495 mg, 1.33 mmol) in methanol (10 mL) at ice bath temperature was added 1 N NaOH (20 mL). The solution was stirred for 3 days, and was then acidified with 10% citric acid. The solution was extracted three times with methylene chloride and the combined extracts were dried via passage through a cotton plug and concentrated. The residue was crystallized from 10:1 hexanes/ethyl actate to provide 318 mg of the title compound as a white powder.

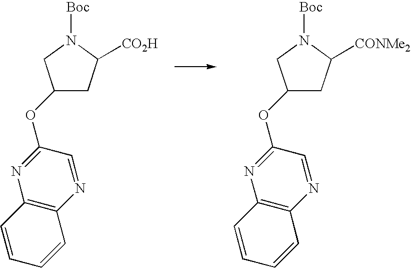

Preparation 37. 2-Dimethylcarbamoyl-4-(quinoxalin-2-yloxy)-pyrrolidine-1-carboxylic acid tert-butyl ester. To 4-(quinoxalin-2-yloxy)-pyrrolidine-1,2-dicarboxylic acid 1-tert-butyl ester (145 mg, 0.404 mmol), triethylamine (43 mg, 0.424 mmol), and dimethylamine (0.505 mmol; 252 uL of a 2 M solution in THF) in methylene chloride (8 mL) was added 50% w/w 1-propanephosphonic acid cyclic anhydride (283 mg, 0.444 mmol). The mixture was stirred at rt for 4 days, and was then quenched with 1 N NaOH and stirred vigorously for 1 h. The organic material was removed, dried through cotton, and concentrated. Silica gel chromatography eluting with methylene chloride/methanol (98:2) gave 115 mg (74%) of the title compound as a colorless oil.

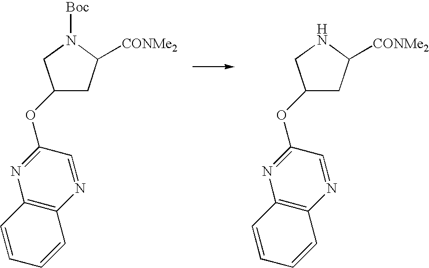

Preparation 38 4-(Quinoxalin-2-yloxy)-pyrrolidine-2-carboxylic acid dimethylamide. Prepared similarly to Preparation 5.

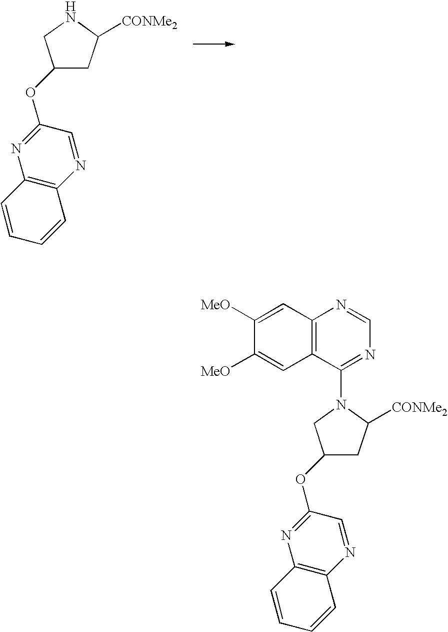

### Example 55

1-(6,7-Dimethoxy-quinazolin-4-yl)-4-(quinoxalin-2-yloxy)-pyrrolidine-2-carboxylic acid dimethylamide. Prepared similarly to Example 1. MS (M+H) m/z=475.3.

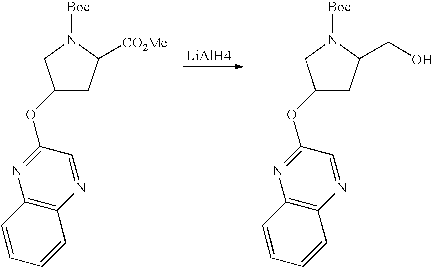

Preparation 39. 2-Hydroxymethyl-4-(quinoxalin-2-yloxy)-pyrrolidine-1-carboxylic acid tert-butyl ester. To 4-(quinoxalin-2-yloxy)-pyrrolidine-1,2-dicarboxylic acid 1-tert-butyl ester 2-methyl ester (956 mg, 2.56 mmol) in 50 mL of tetrahydrofuran at −78° C. was added dropwise a 1 M solution of lithium aluminum hydride in ether (5.12 mL, 5.12 mmol). The mixture was stirred while slowly warming to 0° C. over 4 h. The well-stirred reaction was by the sequential addition of 195 uL water, 195 uL 1 N NaOH, and 585 uL of water. The resultant suspension was stirred at rt over night, and the solids were removed via filtration through celite. Concentration of the filtrate provided 980 mg of the title compound as a pale yellow foam.

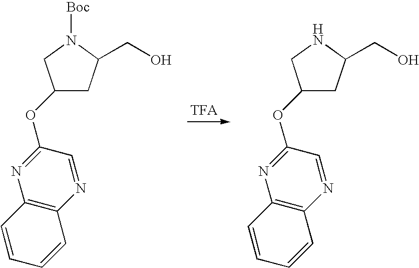

Preparation 40. [4-(Quinoxalin-2-yloxy)-pyrrolidin-2-yl]-methanol trifluoroacetate. A sample of 2-hydroxymethyl-4-(quinoxalin-2-yloxy)-pyrrolidine-1-carboxylic acid tert-butyl ester (500 mg, 1.45 mmol) was stirred in methylene chloride (40 mL)and trifluoroacetic acid (4 mL) at rt for 2 h. The mixture was then concentrated under vacuum to yield the title compound as a purple oil.

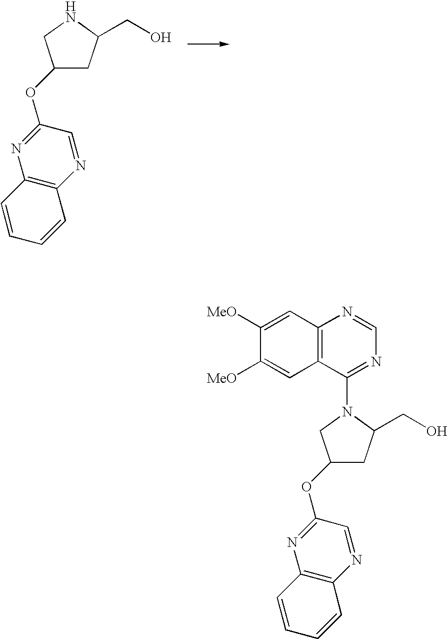

### Example 56

[1-(6,7-Dimethoxy-quinazolin-4-yl)-4-(quinoxalin-2-yloxy)-pyrrolidin-2-yl]-methanol hydrochloride. A mixture of [4-(quinoxalin-2-yloxy)-pyrrolidin-2-yl]-methanol trifluoroacetate (355 mg, 1.45 mmol), 4-chloro-6,7-dimethoxyquinazoline (326 g, 1.45 mmol), and potassium carbonate (600 mg, 4.35 mmol) in tetrahydrofuran (50 mL)and water (25 mL) was heated at reflux for 16 h, and then was concentrated, diluted with saturated sodiumbicarbonate, and extracted three times with methylene chloride. The combined extracts were dried over magnesium sulfate, filtered and concentrated. Silica gel chromatography eluting with methylene chloride/methanol (9:1) provided 314 mg of a yellow oil. The oil was dissolved in 2-propanol, and 1.0 equivallents of concentrated HCl was added. The mixture was concentrated and the residue crystallized from ethyl acetate/isopropanol to provide 197 mg of the title compound as an off-white powder. MS (M+H) m/z=434.2.

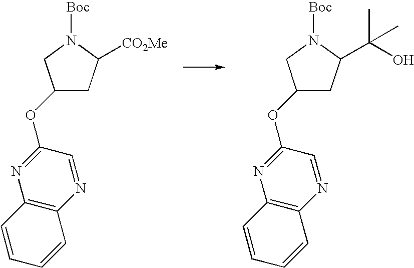

Preparation 41. 2-(1-Hydroxy-1-methyl-ethyl)-4-(quinoxalin-2-yloxy)-pyrrolidine-1-carboxylic acid tert-butyl ester. To a stirred mixture of 4-(quinoxalin-2-yloxy)-pyrrolidine-1,2-dicarboxylic acid 1-tert-butyl ester 2-methyl ester (1.01 g, 2.61 mmol) in THF (25 mL) at −78° C. was added a solution of 3.0 M methyl magnesium bromide in diethyl ether (1.00 mL), dropwise. The mixture was stirred for 1 h, and then warmed to rt for 6 h. The mixture was then partitioned between ether and water, and the organic portion was dried over magnesium sulfate, filtered, and concentrated. Silica gel chromatography eluting with hexanes/ethyl acetate (3:2) provided 520 mg (51%) of the title compound as a pale yellow foam.

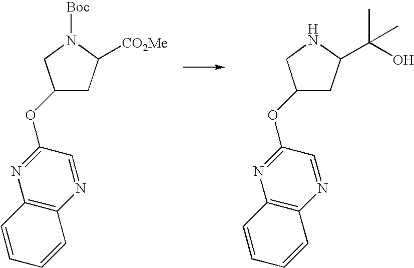

Preparation 42. 2-[4-(Quinoxalin-2-yloxy)-pyrrolidin-2-yl]-propan-2-ol. Prepared similarly to Preparation 5.

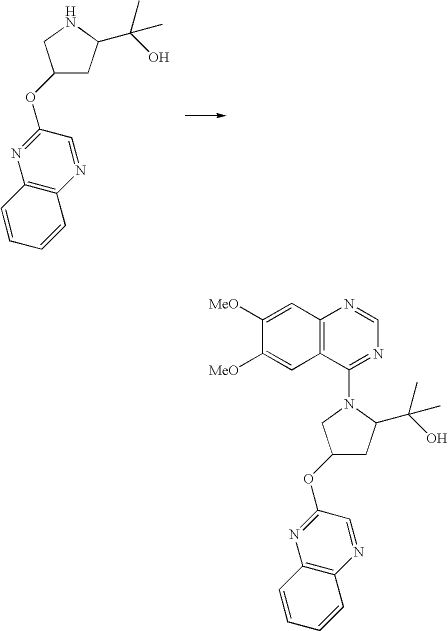

### Example 57

2-[1-(6,7-Dimethoxy-quinazolin-4-yl)-4-(quinoxalin-2-yloxy)-pyrrolidin-2-yl]-propan-2-ol hydrochloride. A mixture of 2-[4-(quinoxalin-2-yloxy)-pyrrolidin-2-yl]-propan-2-ol, 4-chloro-6,7-dimethoxyquinazoline, and potassium carbonate was stirred in tetrahydrofuran (10 mL) and water (5 mL) at 70° C. for 16 h. Toluene was added and the mixture was warmed to 100° C. for 6 days. The mixture was concentrated under vacuum and the residue was diluted with satd. NaHCO₃ and extracted three times with methylene chloride. The combined extracts were dried via passage through a cotton plug and concentrated. Silica gel chromatography eluting with methylene chloride/methanol (9:1) provided 250 mg of a light brown solid. This was suspended in ethyl acetate and the solids were filtered away. The filtrate was concentrated and again chromatographed, eluting with ethyl acetate/methanol (95:5) to provide 97 mg of a pale yellow oil. The oil was dissolved in 2-propanol, and 1.0 equivallents of HCl was added. Concentration yielded the title compound as a yellow powder. MS (M+H) m/z=462.3.

The invention described and claimed herein is not to be limited in scope by the specific embodiments herein disclosed, since these embodiments are intended as illustrations of several aspects of the invention. Any equivalent embodiments are intended to be within the scope of this invention. Indeed, various modifications of the invention in addition to those shown and described herein will become apparent to those skilled in the art from the foregoing description. Such modifications are also intended to fall within the scope of the appended claims.

What is claimed:

1. A compound of the formula I: 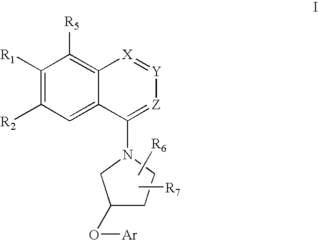
or a pharmaceutically acceptable salt, solvate or prodrug thereof,
wherein X, Y and Z are each independently CH or N with the proviso that at least one or two of X, Y and Z are N, but not all three, and with the proviso that Y and Z are not both N;
wherein R₁, R₂ and R₅ are independently H, halogen, C≡N, —COOH, —COOR₃, —CON R₃R₄, COR₃, —NR₃R₄, —NHCOR₃, —OH, (C₆-C₁₀)aryl, 5 to 7 membered heteroaryl, (C₁-C₆)alkyl, (C₁-C₆)haloalkyl, (C₂-C₆)alkenyl, (C₂-C₆)alkynyl, —O—(C₁-C₆)alkyl, —O—(C₂-C₆)alkenyl or (C₃-C₈)cycloalkyl; or, when R₁, R₂ and R₅ are independently —O—(C₁-C₆)alkyl, —O—(C₂-C₆)alkenyl, (C₁-C₆)alkyl, (C₂-C₆)alkenyl or (C₂-C₆)alkynyl, R₁ and R₂ or R₁ and R₅ may optionally be connected to form a 5 to 8 membered ring;
wherein R₃ and R₄ are independently H, (C₁-C₆)alkyl or (C₆-C₁₀)aryl said aryl optionally substituted with one or more (C₁-C₆)alkyl groups;
wherein R₆ and R₇ are each independently H, halogen, —COOR₃, —CONR₃R₄,—COR₄, NR₃R₄, —NHCOR₃, —OH, —(C₁-C₆)alkylene-OH, —HNCOOR₃, —CN, —HNCONHR₄, (C₁-C₆)alkyl, (C₂-C₆)alkoxy, C₆-C₁₀ aryl or 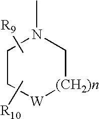
wherein n is 0 or 1;
W is carbon, oxygen or NR₈, wherein R₈ is hydrogen or (C₁-C₆)alkyl, and when W is carbon, it may be optionally substituted by halogen, —C≡N, —COOH, —COOR₃, —CONR₃R₄, —COR₃, —NR₃R₄, —NHCOR₃, —OH, (C₆-C₁₀)aryl, 5 to 7 membered heteroaryl, (C₁-C₆)alkyl, C₆)haloalkyl (C₂-C₆)alkenyl, (C₂-C₆)alkynyl, -O-(Cl-C₆)alkyl, -O-(C₂-C₆)alkenyl or (C₃-C₈)cycloalkyl;
wherein R₉ and R₁₀ are independently hydrogen or (C₁-C₈)alkyl;
or R₉ and R₁₀ may optionally combine to form a cyclic ring;
wherein Ar is phenyl, naphthyl, or a 5- to 6-membered heteroaryl ring, which heteroaryl is optionally fused to a benzo group, and which heteroaryl contains from one to four heteroatoms selected from oxygen, nitrogen and sulfur, with the proviso that said heteroaryl ring cannot contain two adjacent oxygen atoms or two adjacent sulfur atoms, and wherein each of the foregoing phenyl, naphthyl, heteroaryl, or benzo-fused heteroaryl rings may optionally be substituted with from one to three substituents independently selected from (C₁-C₈)alkyl, chloro-, bromo-, iodo, fluoro-, (C₁-C₈)hydroxyalkyl-, (C₁-C₈)alkoxy-(C₁-C₈)alkyl-, (C₃-C₈)hydroxycycloalkyl-, (C₃-C₈)cycloalkyl, (C₃-C₈)cycloalkoxy-, (C₁-C₈)alkoxy-(C₃-C₈)cycloalkyl-, (3-8 membered)heterocycloalkyl, hydroxyl(3-8 membered)heterocycloalkyl, and (C₁-C₈)alkoxy-(3-8 membered)heterocycloalkyl, wherein said alkyl, alkoxy and cycloalkyl may be optionally substituted with 1 to 3 halos and wherein each (C₃-C₈)cycloalkyl or heterocycloalkyl moiety may be independently substituted with from one to three (C₁-C₆)alkyl or benzyl groups; or
wherein Ar is a 5- to 6-membered heteroaryl ring, which heteroaryl is fused to an imidazo, pyrido, pyrimido, pyrazo, pyridazo, or pyrrolo group, and which heteroaryl contains from one to four heteroatoms selected from oxygen, nitrogen and sulfur, with the proviso that said heteroaryl ring cannot contain two adjacent oxygen atoms or two adjacent sulfur atoms, and wherein each of the foregoing fused heteroaryl rings may optionally be substituted with from one to three substituents independently selected from (C₁-C₈)alkyl, chloro-, bromo-, iodo, fluoro-, halo(C₁-C₈)alkyl, (C₁-C₈)hydroxyalkyl-, (C₁-C₈)alkoxy-(C₁-C₈)alkyl-, (C₃-C₈)hydroxycycloalkyl-, (C₃-C₈)cycloalkyl, (C₃-C₈)cycloalkoxy-, (C₁-C₈)alkoxy-(C₃-C₈)cycloalkyl-, (3-8 membered)heterocycloalkyl, hydroxyl(3-8 membered)heterocycloalkyl, and (C₁-C₈)alkoxy-(3-8 membered)heterocycloalkyl, wherein each (C₃-C₈)cycloalkyl or heterocycloalkyl moiety may be independently substituted with from one to three (C₁-C₆)alkyl or benzyl groups; or
when Ar is phenyl, naphthyl, or heteroaryl ring, each ring may be optionally substituted with one to three substituents independently selected from (a) lactone formed from —(CH₂)ₜOH with an ortho —COOH, wherein t is one, two or three; (b) —CONR₁₄R₁₅, wherein R₁₄ and R₁₅ are independently selected from (C₁-C₈)alkyl and benzyl, or R₁₄ and R₁₅ together with the nitrogen to which they are attached form a 5- to 7-membered heteroalkyl ring that may contain from zero to three heteroatoms selected from nitrogen, sulfur and oxygen in addition to the nitrogen of the —CONR₁₄R₁₅ group, wherein when any of said heteroatoms is nitrogen it may be optionally substituted with (C₁-C₈)alkyl or benzyl, with the proviso that said ring cannot contain two adjacent oxygen atoms or two adjacent sulfur atoms; or (c) —(CH₂)ᵥNCOR₁₄R₁₅ wherein v is zero, one, two or three and —COR₁₄R₁₅ taken together with the nitrogen to which they are attached form a 4- to 6-membered lactam ring.

2. The compound of claim 1 wherein either X and Y or X and Z are each N.

3. The compound of claim 2 wherein X and Z are each N, R₁ and R₂ are each —O—(C₁-C₄)alkyl and R₅ is H.

4. The compound of claim 1 wherein Ar is phenyl substituted with one to three substituents independently selected from the group consisting of —O—(C₁-C₅)alkyl, —(C₁-C₅)alkyl, CN, hydroxyl, phenyl and —O—(C₁-C₅)alkyl substituted with 1 to 3 halogens.

5. The compound of claim 4 wherein Ar is phenyl substituted by trifluoromethoxy.

6. The compound of claim 1 wherein Ar is naphthyl or naphthyl substituted by —O—(C₁-C₅)alkyl.

7. The compound of claim 1 wherein Ar is quinoxalinyl, quinolinyl or isoquinolinyl.

8. The compound of claim 7 wherein Ar is quinoxalinyl.

9. The compound of claim 1 wherein one or both of R₆ and R₇ is methoxy.

10. The compound of claim 1 wherein one or both of R₆ and R₇ is —N R₃R₄.

11. The compound of claim 10 wherein R₃ and R₄ are each independently (C₁-C₃)alkyl.

12. The compound of claim 1 wherein said heteroaryl group is a heteroaryl or benzo-fused heteroaryl group selected from pyridinyl, pyridazinyl, imidazolyl, pyrimidinyl, pyrazolyl, triazolyl, pyrazinyl, quinolyl, isoquinolyl, tetrazolyl, furyl, thienyl, isoxazolyl, thiazolyl, oxazolyl, isothiazolyl, pyrrolyl, quinolinyl, isoquinolinyl, indolyl, benzimidazolyl, benzofuranyl, cinnolinyl, indazolyl, indolizinyl, phthalazinyl, pyridazinyl, triazinyl, isoindolyl, purinyl, oxadiazolyl, thiazolyl, thiadiazolyl, furazanyl, benzofurazanyl, benzothiophenyl, benzotriazolyl, benzothiazolyl, benzoxazolyl, quinazolinyl, quinoxalinyl, naphthyridinyl, dihydroquinolyl, tetrahydroquinolyl, dihydroisoquinolyl, tetrahydroisoquinolyl, benzofuryl, furopyridinyl, pyrolopyrimidinyl, and azaindolyl.

13. The compound of claim 3 wherein R₁ and R₂ are each methoxy.

14. A compound according to claim 1 selected from the group consisting of:
4-[3-Allyl-4-(quinoxalin-2-yloxy)-pyrrolidin-1-yl]-6,7-dimethoxy-quinazoline;
6,7-Dimethoxy-4-[3-propyl-4-(quinoxalin-2-yloxy)-pyrrolidin-1-yl]-quinazoline;
1-(6,7-Dimethoxy-quinazolin-4-yl)-3-methyl-4-(quinoxalin-2-yloxy)-pyrrolidine-3-carboxylic acid ethyl ester;
6,7-Dimethoxy-4-[3-methyl-3-(quinoxalin-2-yloxy)-pyrrolidin-1-yl]-quinazoline;
[1-(6,7-Dimethoxy-quinazolin-4-yl)-4-(quinoxalin-2-yloxy)-pyrrolidin-3-yl]-isopropyl-methyl-amine;
[1-(6,7-Dimethoxy-quinazolin-4-yl)-4-(quinoxalin-2-yloxy)-pyrrolidin-3-yl]-diethyl-amine;
[1-(6,7-Dimethoxy-quinazolin-4-yl)-4-(quinoxalin-2-yloxy)-pyrrolidin-3-yl]-ethyl-methyl-amine;
[1-(6,7-Dimethoxy-quinazolin-4-yl)-4-(quinoxalin-2-yloxy)-pyrrolidin-3-yl]-dimethyl-amine;
[1-(6,7-Dimethoxy-quinazolin-4-yl)-4-(quinolin-2-yloxy)-pyrrolidin-3-yl]-dimethyl-amine;
[1-(6,7-Dimethoxy-quinazolin-4-yl)-4-(quinolin-3-yloxy)-pyrrolidin-3-yl]-dimethyl-amine;
6,7-Dimethoxy-4-[4′-(quinoxalin-2-yloxy)-[1,3′]bipyrrolidinyl-1′-yl]-quinazoline;
6,7-Dimethoxy-4-[3-morpholin-4-yl-4-(quinoxalin-2-yloxy)-pyrrolidin-1-yl]-quinazoline;
6,7-Dimethoxy-4-[3-(4-methyl-piperazin-1-yl)-4-(quinoxalin-2-yloxy)-pyrrolidin-1-yl]-quinazoline;
[1-(6,7-Dimethoxy-quinazolin-4-yl)-4-(quinoxalin-2-yloxy)-pyrrolidin-3-yl]-methyl-amine;
N-[1-(6,7-Dimethoxy-quinazolin-4-yl)-4-(quinoxalin-2-yloxy)-pyrrolidin-3-yl]-N-methyl-acetamide;
6,7-Dimethoxy-4-[3-(quinoxalin-2-yloxy)-pyrrolidin-1-yl]-quinazoline;
4-[3-(4-Ethoxy-phenoxy)-pyrrolidin-1-yl]-6,7-dimethoxy-quinazoline;
6,7-Dimethoxy-4-[3-(naphthalen-2-yloxy)-pyrrolidin-1-yl]-quinazoline;
4-[3-(4-tert-Butyl-phenoxy)-pyrrolidin-1-yl]-6,7-dimethoxy-quinazoline;
4-[1-(6,7-Dimethoxy-quinazolin-4-yl)-pyrrolidin-3-yloxy]-benzonitrile;
6,7-Dimethoxy-4-[3-(4-trifluoromethoxy-phenoxy)-pyrrolidin-1-yl]-quinazoline;
4-[3-(3-Ethoxy-phenoxy)-pyrrolidin-1-yl]-6,7-dimethoxy-quinazoline;
4-[3-(3,4-Dimethoxy-phenoxy)-pyrrolidin-1-yl]-6,7-dimethoxy-quinazoline;
4-[3-(3-Isopropoxy-phenoxy)-pyrrolidin-1-yl]-6,7-dimethoxy-quinazoline;
4-[3-(Indan-5-yloxy)-pyrrolidin-1-yl]-6,7-dimethoxy-quinazoline;
6,7-Dimethoxy-4-[3-(quinolin-6-yloxy)-pyrrolidin-1-yl]-quinazoline;
N4-[3-(Biphenyl-3-yloxy)-pyrrolidin-1-yl]-6,7-dimethoxy-quinazoline;
6,7-Dimethoxy-4-[3-(2-methyl-quinolin-6-yloxy)-pyrrolidin-1-yl]-quinazoline;
6,7-Dimethoxy-4-[3-(7-methoxy-naphthalen-2-yloxy)-pyrrolidin-1-yl]-quinazoline;
6,7-Dimethoxy-4-[3-(6-methoxy-naphthalen-2-yloxy)-pyrrolidin-1-yl]-quinazoline;
6,7-Dimethoxy-4-[3-(quinolin-7-yloxy)-pyrrolidin-1-yl]-quinazoline;
6,7-Dimethoxy-4-[3-(naphthalen-1-yloxy)-pyrrolidin-1-yl]-quinazoline;
4-[3-(Isoquinolin-3-yloxy)-pyrrolidin-1-yl]-6,7-dimethoxy-quinazoline;
4-[3-(Isoquinolin-7-yloxy)-pyrrolidin-1-yl]-6,7-dimethoxy-quinazoline;
6,7-Dimethoxy-4-[3-(pyridin-2-yloxy)-pyrrolidin-1-yl]-quinazoline;
6,7-Dimethoxy-4-[3-(pyridin-3-yloxy)-pyrrolidin-1-yl]-quinazoline;
6,7-Dimethoxy-4-[3-(pyridin-4-yloxy)-pyrrolidin-1-yl]-quinazoline;
4-[3-(5-Chloro-pyrimidin-2-yloxy)-pyrrolidin-1-yl]-6,7-dimethoxy-quinazoline;
3-[1-(6,7-Dimethoxy-quinazolin-4-yl)-pyrrolidin-3-yloxy]-quinoxaline-6-carbonitrile acid tert-butyl ester;
6,7-Dimethoxy-4-[3-methoxy-4-(quinoxalin-2-yloxy)-pyrrolidin-1-yl]-quinazoline.tetrahydrofuran(pyrrolidin-3-yloxy]-quinoxaline.;
1-(6,7-Dimethoxy-quinazolin-4-yl)-4-(quinoxalin-2-yloxy)-pyrrolidin-3-ol;
[4-Benzyl-1-(6,7-dimethoxy-quinazolin-4-yl)-pyrrolidin-3-yl]-dimethyl-amine;
6,7-Dimethoxy-4-[3-(quinoxalin-2-yloxy)-pyrrolidin-1-yl]-cinnoline;
6,7-Dimethoxy-4-[4′-(quinoxalin-2-yloxy)-[1,3′]bipyrrolidinyl-1′-yl]-cinnoline;
[1-(6,7-Dimethoxy-cinnolin-4-yl)-4-(quinolin-2-yloxy)-pyrrolidin-3-yl]-ethyl-methyl-amine;
[1-(6,7-Dimethoxy-cinnolin-4-yl)-4-(quinolin-2-yloxy)-pyrrolidin-3-yl]-diethyl-amine;
6,7-Dimethoxy-4-[3-morpholin-4-yl-4-(quinoxalin-2-yloxy)-pyrrolidin-1-yl]-cinnoline;
[1-(6,7-Dimethoxy-cinnolin-4-yl)-4-(quinoxalin-2-yloxy)-pyrrolidin-3-yl]-diethyl-amine;
[1-(6,7-Dimethoxy-cinnolin-4-yl)-4-(quinoxalin-2-yloxy)-pyrrolidin-3-yl]-ethyl-methyl-amine;
[1-(6,7-Dimethoxy-cinnolin-4-yl)-4-(quinolin-2-yloxy)-pyrrolidin-3-yl]-dimethyl-amine;
6,7-Dimethoxy-4-[3-morpholin-4-yl-4-(quinolin-2-yloxy)-pyrrolidin-1-yl]-cinnoline;
6,7-Dimethoxy-4-[4′-(quinolin-2-yloxy)-[1,3′]bipyrrolidinyl-1′-yl]-cinnoline;
4-[3-(4a,5,6,7,8,8a-Hexahydro-quinoxalin-2-yloxy)-pyrrolidin-1-yl]-6,7-dimethoxy-quinazoline;
1-(6,7-Dimethoxy-quinazolin-4-yl)-4-(quinoxalin-2-yloxy)-pyrrolidine-2-carboxylic acid dimethylamide;
[1-(6,7-Dimethoxy-quinazolin-4-yl)-4-(quinoxalin-2-yloxy)-pyrrolidin-2-yl]-methanol hydrochloride; and
2-[1-(6,7-Dimethoxy-quinazolin-4-yl)-4-(quinoxalin-2-yloxy)-pyrrolidin-2-yl]-propan-2-ol hydrochloride.

15. A pharmaceutical composition for treating psychotic disorders, delusional disorders and drug induced psychosis; anxiety disorders, movement disorders, mood disorders, neurodegenerative disorders and drug addiction, comprising an amount of a compound of formula I according to claim 1 effective in treating said disorder or condition.

16. A method of treating a disorder selected from psychotic disorders, delusional disorders and drug induced psychosis; anxiety disorders, movement disorders, mood disorders, and neurodegenerative disorders, which method comprises administering an amount of a compound of claim 1 effective in treating said disorder.

17. The method of claim 16, wherein said disorder are, selected from the group consisting of: dementia, Alzheimer's disease, multi-infarct dementia, alcoholic dementia or other drug-related dementia, dementia associated with intracranial tumors or cerebral trauma, dementia associated with Huntington's disease or Parkinson's disease, or AIDS-related dementia; delirium; amnestic disorder; post-traumatic stress disorder; mental retardation; a learning disorder, for example reading disorder, mathematics disorder, or a disorder of written expression; attention-deficit/hyperactivity disorder; age-related cognitive decline, major depressive episode of the mild, moderate or severe type; a manic or mixed mood episode; a hypomanic mood episode; a depressive episode with atypical features; a depressive episode with melancholic features; a depressive episode with catatonic features; a mood episode with postpartum onset; post-stroke depression; major depressive disorder; dysthymic disorder; minor depressive disorder; premenstrual dysphoric disorder; post-psychotic depressive disorder of schizophrenia; a major depressive disorder superimposed on a psychotic disorder comprising a delusional disorder or schizophrenia; a bipolar disorder comprising bipolar I disorder, bipolar II disorder, cyclothymic disorder, Parkinson's disease; Huntington's disease; dementia, Alzheimer's disease, multi-infarct dementia, AIDS-related dementia, Fronto temperal Dementia; neurodegeneration associated with cerebral trauma; neurodegeneration associated with stroke; neurodegeneration associated with cerebral infarct; hypoglycemia-induced neurodegeneration; neurodegeneration associated with epileptic seizure; neurodegeneration associated with neurotoxin poisoning; multi-system atrophy, paranoid, disorganized, catatonic, undifferentiated or residual type; schizophreniform disorder; schizoaffective disorder of the delusional type or the depressive type; delusional disorder; substance-induced psychotic disorder, psychosis induced by alcohol, amphetamine, cannabis, cocaine, hallucinogens, inhalants, opioids, or phencyclidine; personality disorder of the paranoid type; and personality disorder of the schizoid type.

18. A compound having the formula 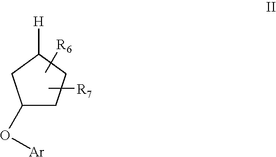
wherein R₆ and R₇ are each independently is H, halogen, —COOR₃, —CONR₃R₄, —COR₄, NR₃R₄, —NHCOR₃, —OH, —HNCOOR₃, -CN, —HNCONH R₄, (C₁-C₆)alkyl, —O(C₂-C₆)alkyl, C₆-C₁₀ aryl or 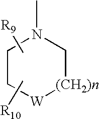
wherein n is 0 or 1;
W is carbon, oxygen or NR₈, wherein R₈ is hydrogen or (C₁-C₆)alkyl, and when W is carbon, it may be optionally substituted by halogen, —C≡N, —COOH, —COOR₃, —CONR₃R₄, —COR₃, —NR₃R₄, —NHCOR₃, —OH, (C₆-C₁₀)aryl, 5 to 7 membered heteroaryl, (C₁-C₆)alkyl, (C₁-C₆)haloalkyl (C₂-C₆)alkenyl, (C₂-C₆)alkynyl, —O—(C₁-C₆)alkyl, —O—(C₂-C₆)alkenyl or (C₃-C₈)cycloalkyl;
wherein R₉ and R₁₀ are independently hydrogen or (C₁-C₈)alkyl;
or R₉ and R₁₀ may optionally combine to form a cyclic ring;
wherein R₃ and R₄ are independently H, (C₁-C₆)alkyl or (C₆-C₁₀)aryl said aryl optionally substituted with one or more (C₁-C₆)alkyl groups;
wherein Ar is phenyl, naphthyl, or a 5- to 6-membered heteroaryl ring, which heteroaryl is optionally fused to a benzo group, and which heteroaryl contains from one to four heteroatoms selected from oxygen, nitrogen and sulfur, with the proviso that said heteroaryl ring cannot contain two adjacent oxygen atoms or two adjacent sulfur atoms, and wherein each of the foregoing phenyl, naphthyl, heteroaryl, or benzo-fused heteroaryl rings may optionally be substituted with from one to three substituents independently selected from (C₁-C₈)alkyl, chloro-, bromo-, iodo, fluoro-, (C₁-C₈)hydroxyalkyl-, (C₁-C₈)alkoxy-(C₁-C₈)alkyl-, (C₃-C₈)hydroxycycloalkyl-, (C₃-C₈)cycloalkyl, (C₃-C₈)cycloalkoxy-, (C₁-C₈)alkoxy-(C₃-C₈)cycloalkyl-, (3-8 membered)heterocycloalkyl, hydroxyl(3-8 membered)heterocycloalkyl, and (C₁-C₈)alkoxy-(3-8 membered)heterocycloalkyl, wherein said alkyl, alkoxy and cycloalkyl may be optionally substituted with 1 to 3 halos and wherein each (C₃-C₈)cycloalkyl or heterocycloalkyl moiety may be independently substituted with from one to three (C₁-C₆)alkyl or benzyl groups; or
wherein Ar is a phenyl, naphthyl, heteroaryl or benzo-fused heteroaryl ring, each said ring may be optionally substituted with one to three substituents independently selected from phenyl, naphthyl and a 5- to 6-membered heteroaryl ring containing from one to four hetero-atoms selected from oxygen, nitrogen and sulfur, with the proviso that said heteroaryl ring cannot contain two adjacent oxygen atoms or two adjacent sulfur atoms, and wherein each independently selected phenyl, naphthyl or heteroaryl substituent may itself be substituted with from one to three (C₁-C₈)alkyl or C₃-C₈ cycloalkyl substituents, wherein examples of heteroaryl groups include, but are not limited to, pyridinyl, pyridazinyl, imidazolyl, pyrimidinyl, pyrazolyl, triazolyl, pyrazinyl, quinolyl, isoquinolyl, tetrazolyl, furyl, thienyl, isoxazolyl, thiazolyl, oxazolyl, isothiazolyl, pyrrolyl, quinolinyl, isoquinolinyl, indolyl, benzimidazolyl, benzofuranyl, cinnolinyl, indazolyl, indolizinyl, phthalazinyl, pyridazinyl, triazinyl, isoindolyl, purinyl, oxadiazolyl, thiazolyl, thiadiazolyl, furazanyl, benzofurazanyl, benzothiophenyl, benzotriazolyl, benzothiazolyl, benzoxazolyl, quinazolinyl, quinoxalinyl, naphthyridinyl, dihydroquinolyl, tetrahydroquinolyl, dihydroisoquinolyl, tetrahydroisoquinolyl, benzofuryl, furopyridinyl, pyrolopyrimidinyl, and azaindolyl;
when Ar is a phenyl, naphthyl or heteroaryl ring, each said ring may be optionally substituted with one to three substituents independently selected from (a) lactone formed from —(CH₂)ₜOH with an ortho —COOH, wherein t is one, two or three; (b) —CONR⁴R¹⁵, wherein R¹⁴ and R¹⁵ are independently selected from (C₁-C₈)alkyl and benzyl, or R¹⁴ and R¹⁵ together with the nitrogen to which they are attached form a 5- to 7-membered heteroalkyl ring that may contain from zero to three heteroatoms selected from nitrogen, sulfur and oxygen in addition to the nitrogen of the —CONR₄R₁₅ group, wherein when any of said heteroatoms is nitrogen it may be optionally substituted with (C₁-C₈)alkyl or benzyl, with the proviso that said ring cannot contain two adjacent oxygen atoms or two adjacent sulfur atoms; or (c) —(CH₂)ᵥNCOR₁₄R₁₅ wherein v is zero, one, two or three and —COR₁₄R₁₅ taken together with the nitrogen to which they are attached form a 4- to 6-membered lactam ring.

19. A process for preparing a compound of the formula 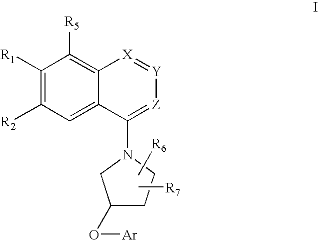
or a pharmaceutically acceptable salt, solvate or prodrug thereof,
wherein X, Y and Z are each independently CH or N with the proviso that at least one or two of X, Y and Z are N, but not all three, and with the proviso that Y and Z are not both N;
wherein R_{1,} R₂ and R₅ are independently H, halogen, C≡N, —COOH, —COOR₃, —CON R₃R₄, COR₃, —NR₃R₄, —NHCOR₃, —OH, (C₆-C₁₀)aryl, 5 to 7 membered heteroaryl, (C₁-C₁₀)alkyl, (C₂-C₆)alkenyl, (C₂-C₆)alkynyl, —O—(C₁-C₆)alkyl, —O—(C₂-C₆)alkenyl or (C₃-C₈)cycloalkyl; or, when R_{1,} R₂ and R₅ are independently —O—(C₁-C₆)alkyl, —O—(C₂-C₆)alkenyl, (C₁-C₆)alkyl, (C₂-C₆)alkenyl or (C₂-C₆)alkynyl, R₁ and R₂ or R₁ and R₅ may optionally be connected to form a 5 to 8 membered ring;
wherein R₃ and R₄ are independently H, (C₁-C₆)alkyl or (C₆-C₁₀)aryl said aryl optionally substituted with one or more (C₁-C₆)alkyl groups;
wherein R₉ and R₇ are each independently H, halogen, —COOR₃, —CONR₃R₄, —COR₄, NR₃R₄, —NHCOR₃, —OH, —(C₁-C₆)alkylene-OH, —HNCOOR₃, —CN, —HNCONHR₄, (C₁-C₆)alkyl, (C₂-C₆)alkoxy, C₆-C₁₀ aryl or 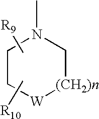
wherein n is 0 or 1;
W is carbon, oxygen or NR₈, wherein R₈ is hydrogen or (C₁-C₆)alkyl, and when W is carbon, it may be optionally substituted by halogen, —C≡N, —COOH, —COOR₃, —CONR₃R₄, —COR₃, —NR₃R₄, —NHCOR₃, —OH, (C₆-C₁₀)aryl, 5 to 7 membered heteroaryl, (C₁-C₆)alkyl, (C₁-C₆)haloalkyl (C₂-C₆)alkenyl, (C₂-C₆)alkynyl, —O—(C₁-C₆)alkyl, —O—(C₂-C₆)alkenyl or (C₃-C₈)cycloalkyl;
wherein R₉ and R₁₀ are independently hydrogen or (C₁-C₈)alkyl;
or R₉ and R₁₀ may optionally combine to form a cyclic ring;
wherein Ar is phenyl, naphthyl, or a 5- to 6-membered heteroaryl ring, which heteroaryl is optionally fused to a benzo group, and which heteroaryl contains from one to four heteroatoms selected from oxygen, nitrogen and sulfur, with the proviso that said heteroaryl ring cannot contain two adjacent oxygen atoms or two adjacent sulfur atoms, and wherein each of the foregoing phenyl, naphthyl, heteroaryl, or benzo-fused heteroaryl rings may optionally be substituted with from one to three substituents independently selected from (C₁-C₈)alkyl, chloro-, bromo-, iodo, fluoro-, (C₁-C₈)hydroxyalkyl-, (C₁-C₈)alkoxy-(C₁-C₈)alkyl-, (C₃-C₈)hydroxycycloalkyl-, (C₃-C₈)cycloalkyl, (C₃-C₈)cycloalkoxy-, (C₁-C₈)alkoxy-(C₃-C₈)cycloalkyl-, (3-8 membered)heterocycloalkyl, hydroxyl(3-8 membered)heterocycloalkyl, and (C₁-C₈)alkoxy-(3-8 membered)heterocycloalkyl, wherein said alkyl, alkoxy and cycloalkyl may be optionally substituted with 1 to 3 halos and wherein each (C₃-C₈)cycloalkyl or heterocycloalkyl moiety may be independently substituted with from one to three (C₁-C₆)alkyl or benzyl groups; or
wherein Ar is a phenyl, naphthyl, heteroaryl or benzo fused heteroaryl ring, each said ring may be optionally substituted with one to three substituents independently selected from phenyl, naphthyl and a 5- to 6-membered heteroaryl ring containing from one to four heteroatoms selected from oxygen, nitrogen and sulfur, with the proviso that said heteroaryl ring cannot contain two adjacent oxygen atoms or two adjacent sulfur atoms, and wherein each independently selected phenyl, naphthyl or heteroaryl substituent may itself be substituted with from one to three (C₁-C₈)alkyl or C₃-C₈ cycloalkyl substituents, wherein examples of heteroaryl groups include, but are not limited to, pyridinyl, pyridazinyl, imidazolyl, pyrimidinyl, pyrazolyl, triazolyl, pyrazinyl, quinolyl, isoquinolyl, tetrazolyl, furyl, thienyl, isoxazolyl, thiazolyl, oxazolyl, isothiazolyl, pyrrolyl, quinolinyl, isoquinolinyl, indolyl, benzimidazolyl, benzofuranyl, cinnolinyl, indazolyl, indolizinyl, phthalazinyl, pyridazinyl, triazinyl, isoindolyl, purinyl, oxadiazolyl, thiazolyl, thiadiazolyl, furazanyl, benzofurazanyl, benzothiophenyl, benzotriazolyl, benzothiazolyl, benzoxazolyl, quinazolinyl, quinoxalinyl, naphthyridinyl, dihydroquinolyl, tetrahydroquinolyl, dihydroisoquinolyl, tetrahydroisoquinolyl, benzofuryl, furopyridinyl, pyrolopyrimidinyl, and azaindolyl; or,
when Ar is a phenyl, naphthyl or heteroaryl ring, each said ring may be optionally substituted with one to three substituents independently selected from (a) lactone formed from —(CH₂)ₜOH with an ortho —COOH, wherein t is one, two or three; (b) —CON R₁₄R₁₅, wherein R₁₄ and R₁₅ are independently selected from (C₁-C₈)alkyl and benzyl, or R₁₄ and R₁₅ together with the nitrogen to which they are attached form a 5- to 7-membered heteroalkyl ring that may contain from zero to three heteroatoms selected from nitrogen, sulfur and oxygen in addition to the nitrogen of the —CON R₁₄R₁₅ group, wherein when any of said heteroatoms is nitrogen it may be optionally substituted with (C₁-C₈)alkyl or benzyl, with the proviso that said ring cannot contain two adjacent oxygen atoms or two adjacent sulfur atoms; or (c) —(CH₂)ᵥNCOR₁₄R₁₅ wherein v is zero, one, two or three and —COR₁₄R₁₅ taken together with the nitrogen to which they are attached form a 4- to 6-membered lactam ring.
comprising reacting a compound of formula III 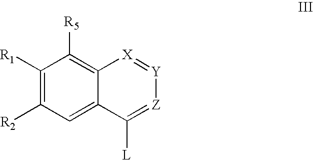
wherein X, Y and Z are each independently CH or N with the proviso that one or two of X, Y and Z are N, but not all three, and with the proviso that Y and Z are not both N;
wherein R₁, R₂ and R₅ are as defined above;
wherein L is a suitable leaving group; with a compound of formula II 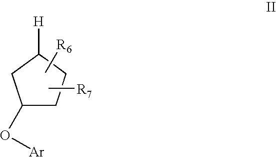
wherein R₆ and R₇ are each independently H, halogen, —COOR₃, —CONR₃R₄, —COR₄, NR₃R₄, —NHCOR₃, —OH, —(C₁-C₆)alkylene-OH, HNCOOR₃, —CN, —HNCONH R₄, (C₁-C₆)alkyl, —O(C₂-C₆)alkyl, phenyl or 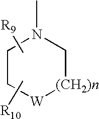
wherein n is 0 or 1;
W is carbon, oxygen or NR₈, wherein R₈ is hydrogen or (C₁-C₆)alkyl, and when W is carbon, it may be optionally substituted by halogen, —C≡N, —COOH, —COOR₃, —CONR₃R₄, —COR₃, —NR₃R₄, —NHCOR₃, —OH, (C₆-C₁₀)aryl, 5 to 7 membered heteroaryl, (C₁-C₆)alkyl, (C₁-C₆)haloalkyl (C₂-C₆)alkenyl, (C₂-C₆)alkynyl, —O—(C₁-C₆)alkyl, —O—(C₂-C₆)alkenyl or (C₃-C₈)cycloalkyl;
wherein R₉ and R₁₀ are independently hydrogen or (C₁-C₈)alkyl;
or R₉ and R₁₀ may optionally combine to form a cyclic ring;
wherein R₃ and R₄ are independently H, (C₁-C₆)alkyl or (C₆-C₁₀)aryl said aryl optionally substituted with one or more (C₁-C₆)alkyl groups;
wherein Ar is hydrogen, phenyl, naphthyl or a 5- to 6-membered heteroaryl ring, optionally fused to a benzo group, containing from one to four heteroatoms in the ring selected from oxygen, nitrogen and sulfur, with the proviso that said ring cannot contain two adjacent oxygen atoms or two adjacent sulfur atoms and wherein each of the foregoing phenyl, naphthyl and heteroaryl rings may optionally be substituted with one to three substituents independently selected from (C₁-C₈)hydroxyalkyl-, (C₁-C₈)alkoxy-(C₁-C₈)alkyl-, —O—(C₁-C₈)alkyl-halo, (C₃-C₈)hydroxycycloalkyl-, (C₃-C₈)cycloalkyl, (C₃-C₈)cycloalkoxy-, (C₁-C₈)alkoxy-(C₃-C₈)cycloalkyl-, heterocycloalkyl, hydroxyheterocycloalkyl, and (C₁-C₈)alkoxy-heterocycloalkyl, wherein each (C₃-C₈)cycloalkyl or heterocycloalkyl moiety may be independently substituted with from one to three (C₁-C₆)alkyl or benzyl groups;
wherein Ar is a phenyl, naphthyl, heteroaryl or benzo-fused heteroaryl ring, each said ring may be optionally substituted with one to three substituents independently selected from phenyl, naphthyl and a 5- to 6-membered heteroaryl ring containing from one to four hetero-atoms selected from oxygen, nitrogen and sulfur, with the proviso that said heteroaryl ring cannot contain two adjacent oxygen atoms or two adjacent sulfur atoms, and wherein each independently selected phenyl, naphthyl or heteroaryl substituent may itself be substituted with from one to three (C₁-C₈)alkyl or C₃-C₈ cycloalkyl substituents, wherein examples of heteroaryl groups include, but are not limited to, pyridinyl, pyridazinyl, imidazolyl, pyrimidinyl, pyrazolyl, triazolyl, pyrazinyl, quinolyl, isoquinolyl, tetrazolyl, furyl, thienyl, isoxazolyl, thiazolyl, oxazolyl, isothiazolyl, pyrrolyl, quinolinyl, isoquinolinyl, indolyl, benzimidazolyl, benzofuranyl, cinnolinyl, indazolyl, indolizinyl, phthalazinyl, pyridazinyl, triazinyl, isoindolyl, purinyl, oxadiazolyl, thiazolyl, thiadiazolyl, furazanyl, benzofurazanyl, benzothiophenyl, benzotriazolyl, benzothiazolyl, benzoxazolyl, quinazolinyl, quinoxalinyl, naphthyridinyl, dihydroquinolyl, tetrahydroquinolyl, dihydroisoquinolyl, tetrahydroisoquinolyl, benzofuryl, furopyridinyl, pyrolopyrimidinyl, and azaindolyl;
wherein Ar is a phenyl, naphthyl or heteroaryl ring, each said ring may be optionally substituted with one to three substituents independently selected from (a) lactone formed from —(CH₂)ₜOH with an ortho —COOH, wherein t is one, two or three; (b) —CONR₁₄R₁₅, wherein R₁₄ and R₁₅ are independently selected from (C₁-C₈)alkyl and benzyl, or R₁₄ and R₁₅ together with the nitrogen to which they are attached form a 5- to 7-membered heteroalkyl ring that may contain from zero to three heteroatoms selected from nitrogen, sulfur and oxygen in addition to the nitrogen of the —CON R₁₄R₁₅ group, wherein when any of said heteroatoms is nitrogen it may be optionally substituted with (C₁-C₈)alkyl or benzyl, with the proviso that said ring cannot contain two adjacent oxygen atoms or two adjacent sulfur atoms; or (c) —(CH₂)ᵥNCOR₁₄R₁₅ wherein v is zero, one, two or three and —CO R₁₄R₁₅ taken together with the nitrogen to which they are attached form a 4- to 6-membered lactam ring.
